SEQUENCE LISTING (68 sequences)

SEQ ID NO: 1 - DNA, 168050 bp, Homo sapiens
cttggcttggctttttttaaagaaaaaaaaaaaaaaaactaagcaggtttctttagaagaaaaagaggaagttcataaagagtgtttgtggtagtaggggtggggtggagtgaggagtgaggaggccgtcacctcccatggaatccttagtcttgggtttgtgaaccacgcatgggggctgttttagcacagctgctaaaataggccaggcctgcaggacatacgggtatgtttagtttctagataacttttgtttttctaaactccttcttccctttcccaaatctggcaagcgttagtcatcttcaactcggcaggaacccacaagtgtgcatgtgtggctcggaggcttcagctggggccccgccctcgtccccaggcgcacactgacacacgcagcccagacccggcccgagcgggctcctgccctcggcgtggcttctctccagccgggagtcccagggccagctagcctcctcccctaaaggggacggcctgtcagcgcagtgccagagtccagcaccgggaggaaagtttcggagtgcggagggagttggggccgccggaggagaagagtctccactcctagtttgttctgccgtcgccgcgtcccagggaccccttgtcccgaagcgcacggcagcgggggggacttcagccctccaggcggggtgggttccaggtccgggtccgaggcgggcgctggaggctcggccccaggccggagaggaactcctttcgcgagctgtcgccgtgggcccgcattgtctgcaggaactctccggaatcgggagggggaggactggatcgcgcttccactgggattcgtcaagagttccggcggcagctgcggcggtggcggagactccctttgtcctctcaggacctccctctctccctccctgtcagctggtgggtcccgctgccgcaggcgccggcgtctcagctgctcgccgccccccaccccagagtgcgtgccgggtgactcccgccacctttgcgaccctcctgagcttaggggactgcgagcgggagggagtctcaggcccccggccgcaggatggtggcggagcggtccccggcccgcagccccgggagctggctgttccccgggctgtggctgttggtgctcagcggtcccggggggctgctgcgcgcccaggagcagccctcctgcagaagagcctttgatctctacttcgtcctggacaagtgagtgtgcgagggaggtccagggtctcctggtgagggcggcactggatcagctggggctgaggctgagatgcgtgtgcgcaccccggggagtggtgttggaaacccgcggaggggtgccctgttgctgtcttctttcaaagggcaagcgcgttcggatctgtgtgggcggagatcagccgggtcgggggtagtgattggagccccgggctacttggctctttggggagttggggtattcactgtttgccagtcttttggcctcttgggaaatgggagtgtggcctcttgcgggccacgctccatcggccacagagtccttacccacctcctcgaccctgctcgtacgcaggctaaagtttgcctggttttatcctaatcatagcgctttcattggagccaggaaaagcctagggttaacatgcccaatgtggtttgcatttcctgcgaagaaaggcttttgtgtttactctgaagagctgggttccactctaaacaattaggcaggtatttacccaccccagaaaacgggctgacttcttcttcttctttttttttttgacggagtcttgctctgggactgacttctttaactctgggtaccaccaaagtttcagctacagatacccttacttagcttgaaaaagaggccaggtgcccctatgttcgtggcattgaagaaggaaccatttcaaaatgtttacttttcagttccgtgttttgtttctctgatcagttcaatttcatctttcaggtctgggagtgtggcaaataactggattgaaatttataatttcgtacagcaacttgcggagagatttgtgaggtatctttcttactttacttttctaggcagtggagaagtagagcaaagtgtagaatttcccttttctgaaaccttttataggaaaaacttttcctgaaatgttacttttaagtgcctcaaggtcgtattgcacacatgctgttattaggacagcccttcagtccttgagggcttatttcatctggatcccagaaacttggctcagactctggggaagcatcttcaaatactattccaaatgtctttttctttccgaattttcatctgttttgaatttcagttatgctttctcataatttatcacattaaaaaaataaattttatgcgaatagtatccataatatatttttctttccacacatttcttagcattatagaggccctcattaaaacaaacaaaaagtgttggaaaacttcaaatgaacatgtgttaataacccgctaccagaaaaggcacttgtgtgtgtgtttaaagcaacgacttgtgtttacacaatgtgtgggtgtgtggttggtttaaaatttaactgttctatatatgcaataagcatcaccaggaaagactggtttcctctcctataaaaagcattactggtatgaaatccagcttactctcttatttagaggtaactaggaattagtggcatctcctatttaaaaataacctcaggacactaggcttttgaggctttttttttccttcttttttttttaaagaaaaaatgcaatgagcaaacacaagtttatcaagataatggaaaggacatgaataggaactgtatcattaaatattgcttctttcagtcttgttagcccaaatgttttctgtaaaacaggcttggatgatcaaaacattttgaaagtcatgttgcaagtaacttcaaacaatctgtagaggcctaggcaatccttgctttcagttattggcagcagtaaagttatccctataaaaatgtatgcctgtccatgttcaatcatcagtcccttgcttaagaagccaagtttaacactcatttctggacagggtctgaggtaaaactgtctaggaaagaaaaacaaacaagatcctatgtccctaaatgtcaaaggcagaacagacagttgtttataaattttctaatttaatccttcaatgtgaaaagggtgtcatgtagattttgatcaaaagcaatgaatcacatcactacaacattttaaagaagcaagctcttatcttaactcattgatctggtttttaagttggaattctataatacacagttgtctggctcaagcctagttgctcaattactaacaatacattgtaggaagtgaataaggggtgactaatgaagagactgacttttaaaatatgtataagcctttgagaggattctgattttcagtttatagattctagtttcatatcttcagttttgaatcttagtagtagttgcagagagtgtccgagaagcaagaaccgttctgtgagagacatgcctaaaacttgaaagatatagctgactggggcacttgttgccaatggaaaatagcatgtttgagacttagaaaataaaaaatgcctaagcagaaagtgatttattgtttctttcacattttccatgactctttaagtagtcttacttcaggatgacttttttatagactcatatcatagttatggtcattaagtcaaactgggtactctccacccataagtgttgataaccataatcactttgaagggcctggccacctttaaaggaaatgttgtagaaaaccaagggaagaggaattttctacgaaagaattttacatcagcttagaatttatctttccagttagtctctctctaaatgcaagtccttatgggtcatggaggagtatgtgagtgctgggaagcgttgactaaatccaaaatgacttatgcaacaatgttaagcagagttcgtctatttacttagggaatgtcatttaaaagaagcctggaccattcagtgagaccaattttaaatgaacctccctaactatacatctgtatcgtatcaatatttctttttaaaaatgttttaactgcctcttttcttattttgcagccctgaaatgagattatctttcattgtgttttcttctcaagcaactattattttgccattaactggagacaggttagtgcattatcatttcactgcaggcttttagtagagatgaattttaaaggcatgattgatatttccaagtggtgattcaggcctctcagtggaatcaagcagatgcacagacaattcagttctctaatagaagggaaaggaggtatgagtaatataaaagagcagttaatctgaaaatcaatatcatttatagtggctgtggaatgtgcagtgaaatctagatccctgtcttagttgtatattcaccacttcctaccttccccggcctctctgttactgtaagtagttctacatttttcttgtttagatataatatattaatcaaccaggaatttgaagaacattgagggggagaaagatatggatccttttggataatttcctagattcagtagacatcctggatttctggtctttctctgaggtccattaatgctgaatatattcacctttataagggaacttagtatatatcaatatgtataccaatatatctctatatctatctacctatctatttaatctccttagtgttttttaaaaggtgtctttttcagaccaatgtcacagtatttcatatgaccatttctgtgtcattcaaacagtgtcaatgacaagttagaaatagtcattgatatattttatgttatttttattttttgagacagagtctctctctgttgcccaggctggagtgcagtggtgctatcttggctcactgcagcccccacctcccaggttcaagcaattctcatgcttcagcctcccgagtagctgggattataggcacatgccaccacacctggccaatttttgtatttttttagtagagacgggggtttcactgtattggccaggttgatcttgaactcctgacctcaagagatctgcctgccttgacctcccaaagttctgggattataggtgtgagccaccacaccccacctcactgacatgttttaaagaatgggatccatagtgggagggagcttttattcttcatttttatgtctactttatttaacagagtatgtaagccagtttttggtcaataaattcttgttgaatgaatgaatgagtggtttcctgttattttgaaataaaatttttctgaactgtagtttcagcaagggaagctggtgtactttgttttgcatgatatgtgaatttctgaaaagttaactgtaaaaacagttttttgtaaattaagttttcccttgaaaaggtcaagaaatcataatatctaaaggaactgggggagagaacttatttataattcacttaattgatttagaaaattatccgtataagattttaatctttgtgcttttagttttccttttcctagagaggaaaatctccctgcagatgctgagtgcagcctaggatcttgtcttcccattgatacttctcctttctttcttctttttaaatttaatttaattttaattttaagttctgggatacatgtgcagggcatgcaggtttgttacataggtaaacgtgtgccgtggtagtttgctgcatctatcaacccatcacatgcattagctatctatcctgatgctctttctcccctgcacccctcacaggccccagtgtgcgttgttcccctccctgtgttcatgtgttctcattgttcagcttccacttacaagaacatgcagtgttgggttttctgttcctgtgttagtttgctgaggataatggccctccttactttcttgtgtggatgagaatgaatctcatcaggagccagagccatggtagtccacaacccgtgtatggcagctttctatgctcagtgatttaacagttctcgcctttatgaaaataaattttctccattgcccagcttaatttccaaagaaacagccttgtatctagccaatccagaaaaaaaagaaccattttaacatttatggcattcctatatatagaaaaacacttagcatcatgccaggcttgtagaaactctcagtcattgatagctgcaaacattgtgccaggttctttgctagacactttgcatatggttcctcagcactttgaagatgaagtgtaggtcactgggtgaccagattggtattgcttagatacgcatggaaggggtttaaatctttaacccacgttagtgattcttctttcatcattggttgaatctaataggggagacactgagataggttacagaagaggcctttctttctttcttttttttttttgtgagacagagtctcgttctgttgcccaggctggagtgcaatggcatgatcttggctcactgcaagctccacctcccaggttcacaccattctcctgcctcagcctcccgcgtagctgagactacaggcacccaccaccacgcccggctaattttttgtattttttagtagagacggggtttcaccgtgttagccgggatggtctcgatctcctgatctcctgacctcgtgatccgtccgcctcggcctcccaaagtgctaggattacaggcgtgagccactgcacctcaccagaagaggcctttctttaaagaggcagcataatgttgcagctaagagtcactagaccaaatctacctgcgtgtaaatcctggctttccaatttactagccaaatggtcaaggataagttgcttaacctctttatgcttcttcttttttcatctatataatgacggcaatgatgatgatgatgatagtaatacctaccccataaaattgttacatgaatgaaagttgatgaatgtgaagtatttagaacagagtctggtatataagttttcaagagataaaggctttatcactattattatcatctggagctaggcagtatacagtattttaatgatcagagaagaatcatgcttacataaagaaaatattgcccttcttaaaaggatttataatattccaaaaggaattttaataacactacttgcaacttgttttatacatagttggttcctctgtctcttcctaaatataaacactttaagttttaataaatttcactatttgtggcatttttaattagtttggggtggcagaaaataacactgagaatgcctaggctagagttccagttttgtcactgactgatttggaaacttggggcaaattgtataacagtgtatcagttttatgtccataaggtgcaggacataaaatcttccatatttgagtttttgtgaagatcaaaataagataatgtatataaagttcctttgtatagcataggacaacctccaataaaggacattattttcttgttctatattgctattttactaattttatttaattaaatcagtaacttctcaagtttctgtggtcctctatatttggatatagggtcacttttcctccattacacacttttaagtgtgaagaaacaatttgcatattatatgcagatcagtaaacaaatgattccacaactattgtgaatctttgtttcaaagcctagaaattggaattctgcatataaactattaaaatcttgatgttatatattacaatcacaaatagatcagagtgtgtgaggaaagcaaaggttaagcaaagaaaaccaaaggttaagcactgcaggtttcacaattttgtttctagcactcataattcttcacaatcttggtaaataagtagctctttcatgtttctcagagtgttgctgaagattcttagtagagatgaggcaagattagaaggtagtaaatacttaccgtagggctgtgtcttctgttatgctttcagcatggcatccggtactatgtaaatgcatgaataaagtaataaacatgccaaaaaatgatgttatgcattttatatactcgtaacttaaaaaccttattcttttaaatactttaatttgaaacactgccttcttttctcttgctctcatgatttttaccctccatatgtctgttagtccattcctccacctacccacccacctacctaaagacttgcttatcttatattcaatgcattgagtactgtggaacctggggtaacacaaagataagtaatctagtgtgtgtgtgagtagaggtatacacatgcacataaatagcccagggatgtattatagggttaggtaagagaaaaatgccaagtgttgattcagtatggaaatacccagcctttcaacagtagagaatttggaatcaagactaattaaaaagtatttttagttattattttgcataattggggatagtgaattaagctcagttctgttaaatattatgctaatactgtaacagagatttagttgaaaacatttattgaacaattattatgcaccaggcagtgtgcaaaatactttccatgcattgcttcacttaatcctcatatgaacttcctgtaatagatactgttattacctcgactttacaaatgaagaaatcaaaactgacaatgataaagcggcttgtccaagcacacacagttaacaagttgcaagcagtatattggctacaataatcttttaaagttaaagccgatattccactctcatgcttaaaacaatttcatcacttagactggaaagccagtgtttcccaaaggcctaaacgccccatcctagtgccccagccagtcctctcctgcaacatcatgtggcacagtctagctcaagtggtcttctttccattccctgaacagactgagcttgtttccacattaggacctttgctctttctttgctgcagatctgcccctagctggctcgttctcctcatttaggtttcatcttacatgccacatcttctgagaagccttgcctggccagcaggtctaaaggaaccccacctctcctccccagtaatctcttacaatactatatttggttatcacattttcaagtggttctgttggttaatttatttttatcttgatgactctaatgtaaacatgaaagcaggagtcctcatttgtcttgctcccccatgtatcccaacacctagaacagagcctgatgctcagtagatgctcaataaatatttgttgaatgagtgattgagctgatatgacttcagaaccttagttattagctgccatcatagattgtctttttttgtgattataccacataataaataatcttatatttgctatttttcttattcctactcttacaaatcagttataaatatttatgactccctttcattacacttgcccccagcctgaaaatgtcttataattgtttgacctgagcacaataatcaattacttggggaaaaaaatcttccgtaggcaaacttgtaggaaattatagaaaagtcacttaattaattttggacaatatacacatttctattatttgaatttttttttctaatttcttaaaagctgaattaaaaatttacttgtggaacaattgttctttatatacagtaagtaacttattaattggtacattcattttagaggaagcttggtagtatctataaaagtctagaaggttcctttcagtaagtctactttttgggactatgtcctactgaaatactagcattagcatggaaaggtaggtctaaatacaaaattggatactgcaacattgtacataatggtgaaaaactgtattcaatctaaatgcttatcaattgatgatactataattaaattagaacatatacattggatggaatactatgcagttattaagaaataggaaagtctgtacatatttacaagagaacatataagagatatactaagtttaaaagcaagtagcagacactcttagtatgattttatttttataaaataaaaagaaaacaaaatccctaactgtttatatggtgaggggggtgtatgtgattgaaaatgcaagaaaagaagtctagagaacatacaacaaattgttaacaatgacactgtcttaggcatgatattgaaggtgggaaaaaccttcacaattttaatttatatatatatatgtggtttgattttatttcaacaagaatgtattgtttataaaaaaaatgctgaaattaatgttactatcttggaatcaaaatggaattcagaggttggaaacaagaagcattcaggaatagagcatattcaaccattactatggagagtcctgaggttttttgttaatgaaaggaccagaattggaccttgggtggaataggcataggagctacatactagctgtgtgacttctaacaaattactcaccctcttgctgcccaattcctgggccagttaataagagtgaattattcctaccatacagcaatcttgagaaaagtataatgtgtgagaaggactaactaaggttctacacataaagagaacacaatgggtatttataactttatcaattcacttttatttctttgcttctatcgcctacctgcaactaactaccttttaattttttctcatttatagtttttcattttttttcatttttcttttcttgctgttgctttgatgattgcataggaaggaggccaaggctaacatggggtttggcagttatcagtggacaccatccttattttcatcatgatgtatcaggattcgagcagatgtttaaagaaatcaaaggactatgctcaaagtgacacagctaagcagctagagccagggttcacacccaggcctgatgctgtgcatgagcacttcaccaccgcactgtattcacactctctgggttctccttctccagttttcaatcagtcagaattagtcacaactcaaaaacatcgtcattccctaaatttattctattcttaaaaaccagtgagggggtgaaaagatgagacaatagttgggttagctaaaaaaaattccattaggcaagtatttactcctgttttgaaaagtctgttttggccacacattgtgctaaacctgaggggtccaggcagggcagcaactcagattttagtgatggacatgttagcagtcactttcgctgaagtcccacatctgttttatgagcatgttctagaaaattaagtacattgttactcatgcctgggagacagacctggcacacggaagtggtaaaatgcaggggttgatttttcatgatttagtaaggcagtgagtgggaaaatggagtaaaagtatgcaaaacgcagcaggcctgaacttcaggtgtgaattgagtaccacattttggtaggtgcaggccaaattggagaatatttttgacaaaattcagattgtacagggtattttgtgagcagatatttctggatctatgactgctaccttggatttgctggtctggactctggaccacagaagccttggcctatgttggcattagaatgacatgttaaataaacaataagcatattttacatagactatagtaagatacagctaaaacccattagttcccaagattttagaatgtattagcaatagcaatgtgtgtctttagaaaaaatctttcactcatttgaaacctttaaacaatttttaaattgaggaacttggtgagaacaaattacttgttagaggttcatccgttaagcagtatgtagggcagtagtatatagtaacacagttaagtagagcaggtatatatgaatgaacatatttctcttcatacagtttttttctatttttcttactcaggttaattttgtggtttattaatggaaggagacaataaaagtatcaaagtttactgagcagttactatatattacatattttactaaattcttcatatgcaatagctcaggtaaagtacaattctaagtgaattctaagtgaattataaatcattagcaattttgtatagtcataaataacatcttgagaacattttcgtttcatattctactcaatctattaaattttaccctaaaagaaacatttcaggatgctgtgtaattggctaattatcataaaatccttatgtattgctacattttgttttggaattactgtttatttcaagcagattaagtctgtggctaataatctattgagagtaaagaaaataatttacttatgatataaatacctatagtattcctgcaattatataaattggcatttgacaacatgagtgtgtgaggctttttgttttagtggtagattatttctttggtggcttccaatgaagcatagaatacacacacacacacacacacacacagaggcacacacacactttttactttttaaaatctgggtctaggcctacgatttatttacttcagtcaatagaatgcagcagacatgaacatgaaactgccagtttgagttccaggcctaagatttattaaggcatggtagtttcctctattgttttcttggaaaccatctgtcatatgaaaactttgactactctgagaccaccatgctaggaagaagcccaacctagccatgtggaggagaatcgagactctggttaacagtcttagctgaactcctaggcaacagccagtatcagcgctggcaccgacttgctaaccatgtgaaggcattccgcttctagccattccagtggctcaactggcagcatgtgaagcatgagaactgctcattcaatgcacagtgccatgagaaacaatcaattttgctgataaccactaattttggggtacactagactccccttatctgaaggagatctgttccaagaaccccagttgatgactgaaacaacaggtagtgccaaaccctatatatactatttttttctatacatacatacctatgataaaacttaatttataatttaggcacagtaagagattaacaaccataactaataacaaaataggacatttataataatatactgtaatgaatccttacagtggccccatgggggagatatttatattcctattttataaatgaagaaacaaggttgagcttggaactaatctctattctgtctataagctcatgcacatcttcatccataccatcccagaaactatttatggtttaagagattatcctatttacccaaaactctcatacagaagcaatgactggcaaaataggtaattcaaaacgcattccaataacacaagaaagttttaaaattaggttagatcagaggttcttaatcctggcagcgcattagaatactctggggagttttcaaaggccagtgataaccctgacccaacacaaacatgtgaattagaatctcttcaaatagagtccagaagagcttttgaaaatcccctcaaatgattccagtgtacagccagggtggagaaccacagtgcatatatagagctttatgtgagtgacccctggactgagtgtgaggaattctcacatcactgtgtagaatcacggtgttcacatacaacagatgctcaacagatgagtgtagactcattgactgaaatcctgaaagagctttgaaatctggaaagttaagacaatgtgcattggaaagttcaagtactgtacagacacatcgtagaaagaaaaagtaggagatactgaaaataaaattatgccttcattgttagttggtcaattcaggattccctgcagactgttttatgacaccatgatttcagtctatctttattttagttataatgccttgcataccttccacatccaaagtgtttcagagaaaagctactgatatggcacagtgcatggcagttgtggtggctattttcccttcccttcctcactcctctcctcttctcccctctccttcccttccctgcccttctcagcaaagaaattagggagatacaaatagagctgaaagtgctaatttcatacattaaaaaattagccaagcctgggcatagtggctgaagtctgtaattccagcactttgtaaagtcaagatgagaggcttgcctgaggccaggagttcaagaccatcctgagcagtatagtgagaccacatctctatagaaaatttaaaagtagcctggtataatggtatatgcctgtagtctcagctacttgggagactgaggtgggagggttgcatgagtccaggggtttgaagttgcagtgagctatgattgtgccagtgcactccagcctgggtgacagagtgaaactctgtctccaatatatatgaagcctctaaatgaaagcgaggaaaacaccatatatatatatatggtgtttgtgtgcctcctctctttctctttttccctccagttgataatggaatttggtcccccaattctcaattaaagaaaaccagggctttaatataacattttgtttgttcaattcctgtctctttcaaaaaagtatttgaggctgcatagaaattaatagtgttgcagaaaattaataaaatataaaaagaagagtgctaaaattttggaaaaggggatgaaacactatgataaccacatgaacttaacagaattttgtgaatgagtaagaaatttactgtgggcttccccagtagtcaggacaagaaataagtttgcatggttgtcaagtgcagaaaagaggaagtacatagcaactacttgggggtgacaaagaaacttctgctttgaaatgttataggggacgatgcgtgtcatgacagcagcatccttgacaactgcttcacaacaaatacaaagtagtttcaatattgctgttccctgtagttgaagacataacttgaaagccaaactcagtgaagctgatcttctaagatgttgaattcaaggagttgaacttatctttataacataacctttctttcttcttgttcttaaaattttagtcattttcgctttcatttagaggcttttttttttaacattacagtggggatgaatgatttctgtttttaacctaccacattattttgctttttcaccgttaactgtttaccaattgaaaaacttaatatttattttcagttgcttgggattcttcaagggtctgagatctgtctgccctgtcttgttttggcctttgtttcccctaaagtgataccttgttgattgataagaggctgacagacttcaggtagttaccctgaagggagtacaaggccaggactcacatgtgtttttcttggctatggaaaaggagcactttcctcttaggatcatgcagctgcaaaagtaatgatgggattgagtggagcttggaatcttatggtgcaggtataggtacagatagaaaaaccgcaaactgctgtctctcttccagtgtaataagctagcagatacctgactgtacaaaaaaaaaccttgaatccacgtttgggaagttcctttagcttaaagtcttgtgctggtgttgtctgggactcagcagcaatggtgagctgatttgaactgaatggagaaggctgtcttatgcaaaatctgtttgtgtaaacttgcatttcacttgttggcagcaaacactaatgctcaaagacacactgccactcatttctgaaaagcaccaaatatttgacttttatctgcattcagacacaatcagtttcaagttgtttctgtagctatcaaacgtaaatattttagatacaacaagcttatgaattacataacttatgctatcatccttggaattataatttttttatgatatattgaaaaatttattgaacacttactttgcaaatgacattataataagtacagagcaggggaatacagcataaataaaagacatggtaatagcctaatgtaaactttcagtgtagttgaaaagtgaatatttaaattaaattagttaaattattagaaagttgaatgtttttaggagagaacttttttttattatggtaaaacatgaatatgagtatgactagaaagcttggggtgactcagaataagagtgtaatgaacttaaagaatggttcagaataggtagactttaaaccaaatatttaaaaagacaattagcaagagttaatagagatgagtgaaccaggaaagagaaggattagggtcataccttgaacattgtcttctgttacctggaaactttatgagatggaaaacttgcctgataatattttttattgcgatataaaaatacaatcaagaagttacttttaaaacgtatataaggctctggtgaatggattatttatttcaattatttgaaacacaatattgcagcatgacaaaatgttttgaaattatttttgacatattttataaccgcatgtgatataaacgtatacacacatgtacctatataaatgttgtgtatggccccagtaagttccttgatgttaagatgatacttcttacaattaatctccttcatctatcatcctcagtaggagtgagtactcttcatgtgttatttacaattttctaattcagtttatcatttcaagaattttacatttaaaaaggcacttcttatcacttctcagccttttggttaagatcaagtgtagtatccattctcaccagtttaaaaaggcattttttttgcttctattgaaaaagtctgaaaacaatatcttaagatatcttcttatcttttaaatttattataaaacaagttttgaatttaatttggataattttgccacattattttacttatcttttaagataattggttttatttattttcgagaggtttcaaatctatattttaaaaactctaaaaattgggacttcccttaactgatacacataaaatagctcttaataatattgtagtcataccccatttagacaaattcagttgggatagtgaattagtcatgatgaaagaaattattctatagtttttaattgctttcattcatagtgtctcttccaaggattcattaccttctaattcatccatctatccatacattcatccatctatctatccatccacctattttttcattcattcatgaaatatgtattaagtacctactatgttcaagggaatgtgcaagggaatgtctggatgggtgagaaggtgtcataaaagtgaataaatcatgagacttacgactctgggagacagtaagaaaaaatgttaatttctgtaaataagggctaaataatgtgctttagcaattcgcagataacaaggaaaatttcccacttttagagagatggagtgagtgtttcagaaatgctttgtcattgaagatgagctactcaatttttgtatttttgtggataagggaaggtatattttactccttttaggaaaacgagtgattgaagatatctgttctcagctcagaaggcctagaatatgcaaagatttttcaagacattgttagcacatcaatgtctatttttggtagttaaaatacagaagttattaaatacttactaaaattacaaaaagtatatgaagttatctgccttcaaagcccatgttctttcctctgcttcctactgaagaaagcttagttacaatactgccatgttttttaagaagtgtttattgttacctttgctctttgctcattttttcttttgtttaatattattttcagaggcaaaatcagtaaaggcttggaggatttaaaacgtgttagtccagtaggagagacatatatccatgaaggactaaagctagaagttattttacatttgtaaatatgggagagaatgctttcttattctgtggtgaaataaaaccctctagcaaagctcagtaacctcattacatatttatcttaaagtttttaaagtgcttttagttgttccagagttgagacaagtagaaaaaattatttactatggtgatgagaaaagttaagatggcttttgccttttaaggaattttccatttcccttgtggttatattaagattaagcagccacagctaaaaatctatataatttaaacccaatagtaactactctgtaatgtgaatatatgcatatttcaaataaaatagttaacacaaattaccctgttttaaaagctagccagtaatattttgattttgtcagacttacaaattcagcttgatggaacatgctggttaaaagttaaaattaaattgtgagttggataattattcttcattttcaggcgaatgaacaaattcagaaagcaggaggcttgaaaacctccagtatcataattgctctgacagatggcaagttggacggtctggtgccatcatatgcagagaaagaggtgagtattgaactgagactgttctctacaccccactgtacatcgctgtgtgtctcggaatgtatagcttgatatttgaataattcactttctccctctcccctctctttttttctctctttccctcctcattctccataggcaaagatatccaggtcacttggggctagtgtttattgtgttggtgtccttgattttgaacaagcacaggtaagttacaagagatctgcaaactttaatctgaaccacataattgtcttaaggatagtttaacaaattcttttcactgatgacattgtgacactggtcgattgttaaagattttttatagaaatggctgtaggttttgagtactaccttaataatgtaagaattttaataggttttgttactgaaaatgaaactttaaatctctcaattgtttggtttggtaaactaatataaggtaaacatttggcttgaaatgatacacatatttctcttaactcaattgacaaaatgttattatattatatatataatatgtagtcatattattataatgttagaatttctattttatttagaatttgactctaatgaaagtccatctaatgaaagtctgatcatttttcttacaagaccatttttttcatgagatttgagggtttatatatttgaaaatgtttcactcatgaagttattttacgtttaaatttttgcttgagcatggcttagactcctgagtcacgctgttgatagctgttagatttcctctgcactgttttgaaattgcacttggggccaacttaaaggtactctgactgatctcaattcattttgtggtaatacatttagagtaacactagtatcatcacctttaattcctgaccttgaacacagatcacgctggtaaattaatcctgtggggtaatttttcagatgaacataactgaaatgtaacgaagtttataaattgttgtatgtgtcagccactccttaattaagtcttcctcttctttctaaagcttgaaagaattgctgattccaaggagcaagttttccctgtcaaaggtggatttcaggctcttaaaggaataattaattctgtgagtatttctctgggggcaggaagggctcataaccttgtatatttttttaatctataagaagtaatcttaatatccagtgcaggtggtcattatctagaggaaagagattaaggaagagatttgtttggtattgcaaacagtgtaattctaaagatgtgaattttgtttgtaactgacatttggtattaattcttgcatgttgggctctgtgagactgggtttgtggtttcaagtttaagattttaagtctcagacactttcaccctttgattctggagttaactgtcttgatatcctgaaagttttataggttttataggagaaatttaaaataaaaatttggattgatgtgtgattactcccttctccaaatatagtatctgttttaactatatcagtcaggcaacttgaactagtggctatttgactacatttgttgttctttccatttaaatgattaatagtttcactagagcacataatatttctgtttggaattcagttgtgctgtctctagtgatcattagaatctaggctatcctaaacagggtttggggaaggagagtgatgaaatgcaaagcaaaactcctgggctttcatagctgaggtttatgtttcattttactgccttttgctacaagcactgggaacagttgctattaagtatttatgatattttggtaaacaccatcctaaaatggaagtgaataatatatattctctaagccaaagcattgaaaaaaaatcttcaggggaaaaaatatcagtttcttctaacatttggttctgggattttaaatttctctttaaagatcatgttgtttagaaacacaaaatgagatttattgtatttgtttttagtagcacgtaggcttttaaaaaaattaatggagcttttcagagaaaacataatttggtttcatcacttatcagtacatctagtggacaaaccaaatgtaagtgcagaatggaacagagattcaattatttttgtagtttacatatattgttgtgtttaggttttagggaatagttgcagtgtaaaaatagtaaataattttatagctggattcaggataaataaaactgatacaactgtgatttaagttttaaaatacaggatacagtactgacaatgtaaaaaggaggataatttgaccctctaaataaacaaatcagtggattgtattttaaatagaattttattttgctctgggatttgtaatgatttttataattatactgggaaggagaaaggacctctctgggctattcctttggatttttttaatatctaaaaggtttagctcagaattacccagatgatggattgtttccaaaactctatttttatgggtaataactacagatttccccttcaaggaattagtagctgagttccagatgacatcgttatcattgttttattatgtttgttggatacatttaatttttctatttttattagggaacctctagaatgtaacttctaagtatggcctttcttgcctaaagttcttgatgaactttaacttaagttatgttgaaagatttagcatgaaattaggaaaatttgcattattttagccacaatttgccaacaaaatctcttgagtgttgtttggagggagatcatgggtatagttttcatgtcaatacagttaaatttcagctatgtgctcacaaagggaagagtaaattattcttaggaaagtcaccttttggggaaacatcgtagtatctggcagagttggcaggtggaaagaaggagccattacaaccaaacctggtttgattaatggttgagatttttatcaacgtgattgatagtttgagatttttgcccactttgtagaaattaaaggcaatgtaaactttcatgtattttcaagggcaaagattgctccatccctcatagtatacaaaacctataattttgccttaaatttgaattggactttataattgcatagtaccatctgatttttaaaaatatctattagatctttaaaacaaatatagtgtttctgtcacatggctaggagagatagaagagactatactctattctttatgatttatcaaactaatgggtgaaattatactgtggaatgtatttacataaagctaagccaatagaagaaataaaaaattttaagtctttcagatatgtggcactatgaaggcagaatatttgctaaattgataattttttttcctgctcattgttttttaaataaatctggcctcattgtcagtgcattaatttttatgaagttaatccttgaaaaaaattgacaacatggtttttattgttttgtttgttctctctcccctctaaaatccaatgaccaagatagtctggtccccttaatgtaagatatcatatattgttaagacatcatttgtctagaattaatttcttaatagcaaagattaagaaagttttattttggagcctttcttcatggtagttaaaaaaaagaagaaacactgtagtagttctgcttttacaactcaaaaatcaaggggctgaaaaaggcctttgttatgaacatcctgcacgttaaaattttaatgccttctctaaaatgtatttgatccagaaaatcacttaagcattctattttaattttggggaaaatgcattgttttagtatttttagatttctatccaggcaaacatggtatgtaacagttttggggaatattgatgtaatttccaatcttagctagttgcatatgaaggtttgtaattcattatctcaagtatatactggaaggaaacatttcttctttttctcctttttttcctcaatgagctcattctttcattagttcatttactgagcccttactattcttaaggcattgtgttatgattagaagaaggatataaacttgaataagacatattctctctaaaggggcttgaaatgaatgattatttgaggtttggagtatggtagtcccctgaaagatttacaaagcattgtggacatttagaggaggaaaatttcagtgagtcaccttaggggataagaaataatcttataacgcaattggtgttaaatgccaatatgcctatagtgtaaggtgacataaagacaggatgggtttagttatgcgaagttatgggaggcctttcattgtgaaatttgaactggactctgacaattgggatagatttaatgagaaacaaagagcacagaacttcaagaactatctgagctgactggctgaggtgtaggctggaatgtggtttggctaggatcatgggaggtatgggggataaacattgacgggtagcgtggattcagattatggagactttcacatacttgttttgagaatgcgctcatatttcataaataagtaggaaattatcgaagattaatttagagcatgggatttatataatggccatgcaggtttaggaagagtactcttataataatgtacaagatgatttggaagatgatgatgtgttataagtattttgctgtcatccttgtgagacacgatgaaaggaagcacagcatagtggagacaaaatgcagttgggaataggaaaacctgggtgtgaattccaactctgtcttgccaagtgacctggggtatgtccggcaatgtaatttctctgagttttaaaaccctcagtcaaatgggaatagtgatttccattttgttgttttattttttaaattaaatgagataacaattgagaatgacctaagttagaagggcttggctttaaatcatttggtagaaagctaggcatggcaggaaactaacccagggctaatgatggggctcctgatccttctgtcgggcagtccactgagagagatgacccccaatctacttaaacaaggtcttaggtacaaaaggacatggcactgacatgtaagaggatcaaatttaggatgtgagtcatgacaacaaggatttttagatagtgtagcacacaaagtcaagcatttggatgtgcgctggtattaaatcctcaagccgtaagatcaggacctagccaacaacttggaaacaaaattcagtattcccatgacaaaagctatgtagaatttctggaccgcacattcagaggagagacacaattcctgtggtgggacttatagtactaattagaaattcagagaagaaactcagtagttttggaagaaacagtggatcatcatagttccttagggattattcttggcactgggattctgtctttcgcaatggaactaggtcaaggatcatgagggaagcacctaataacctctttattctggcaagcatcatgtcaggaacagggtggggctaaacctccaggtataaaatctttgactcttgttgagctgggctccgagagacatggcaaatattagctgtcaaatgttaattattgttagtattaggatgggcagtgaaactggaagtaaaacgttaaatttgggaagcattaaaatattagtagaatttgagagctcactggctatagggttcaagagacagagagaggagtcaaaagtaacttgggcattctgtccctgactaagatgaatgactaagaatagagcaaaaactgtttttacagaggggacaagagggttattgtacaaaatggaagaaggcatagggcagagggggaagttgatttgagcttgagaacaaaagcccagtgggggatatcctttagggagttagaggccaggtggaatggaaaggctagaaaaccctactgtcattatgcttcctcattttctttctcccctcccattgtccacagacacaagcacatatatatattctctttttcactttccacttcagattatctttaaatccttggaatgttaaattatttttgtgttcttgggaaggtgattgcatgacttatatttgaaaatgaaattttatttgaactagtaaaggtcagactgagatgtgagtaccctgtgggttctagttcaatgtttcataatgcagactatggaagataacattaaaaataaaagaaaaccacagattggtgaataatatggaaagaataaaaatcaaaatgttctgtgcccttccttgtgtaaaaacaggaccatgggatgtttcactatttggaattattcccacattttttttaagaatacctcagaaagtattaagcagaaagaaataaatcagttgtgaagcatgttttataaataacccttgtccaaaagaattgagaacactacatcctaggtgatggtagttaatgccagagtttctaagtttttaatgggaaagtatacaagcataaataggaagggaacgatcatccatacatttaggacatttaaataaacctgaattagatagtttctaaagggaaaagtgaaatttcaaaaagtacctacattcatagtgctggagatataagcttcatttcacttttggagtggaatttttttctcagcaatgtctgttgttgtttgttcttagaaaaatatctaagaagaaaagtgtctaacactatattttatattgtctaacactatattttaatggaatgagaagacattgcaaacacattttctcttaaggcaaatcattttcatagaagagggaacctaataaacactctgataataccttagcttaagtgattttagcacactctgctatgaatgttgaccacttagttgtgaaaacttctatttgtaaaattacggtttgatgaaaaacctgaagcctgagcaactgtaaagatacattcataggagaaacaaaaatggtaggacttactcaaaactggaaaccaattttcaggactcctgttaaaaaggactggctataatgtaagaataactgtatatcagattattctaggcttctctttgtccaaggatcattagcgctgaatgtagatgaggtatttcattataataactttcacattaatggtatgaaggttgaaattgtattgccccaactcctgtttttgtttttttttttttttacctaatgcttaatctagtgttgggtaagtagtcattgcttaattcatatttctgaattataagtaaataaaatctatgcaagtttttgaaaatattcttatatttttataatatagttatggtatattggagcattaaaaattaacctctgattccatgtgcattagataaataaataaaatgaataaaaatctatgcaagttcttaaaaatgtttttatatttttctaatgtagttatggtatatttgaacattagataattaacccctgatttcatataaatttgatttcatcctcatatttatgtttcactgtattaagactcttttgatttcacatgacaagaactcagttaataatagtttaaggaaaagaaaaatagaatgttactggcctgtaactaggtggtccagagagggaaattacctcaggtacagctggatccaaggaatcaatcatgttgtcagggtcttctctctcagtaagttttgcctatgtttctatttgcctcattccttgtttagatgaatggtggtgaactgtaattgcaaaaggaggcttacatggtcctcagcaagtatattggaaagtgaggggtgttgtctttgcatcagtgtatagggtcccagataagaactctaattgattcggttgagtcatgagctcattcctgtaccaattctgttgggggcaagggagagagggagaagaaggcactgtgattcacagcctcactaggaccacatggagtggaaaagagtttcccaaatagaaaggagcactattcatagaagtgtaagggtgcaaaagtgtccagggtaggaacaaaaaaatcagtagtgatgacagctcactatattcaaatacatttaaaatattttgacatatattctaagaagacaccccattcctccgaaatcccacttgataataaacatagaaagtttcctttaaattattcattttattaactgatcttattttggcctctactgtccaaagctgtatttgtatttgcatgaaaatattccctaataatattattattttcttttttttaaaaaaaatttggttgggattgtagtttaagaaaattaaatccactttgaataaataagtacagctttgtgatagtttgtgagaaaactaaactaaactgatgacatttggttacacagataaaatgaaattccaaattttaatttcttaacattaacctgttggactataaaaggtgtcctacagaggcttacctgctcttaaaagcagatttaattgagtggaaggaaattgaacaggtgcagaaaatggttaatatttgtcaccaaatgtaccatttggtgtacattatgaggaaaaatgtacaatttctcaattatacttcattttctccaaatataataattctcagttgattcagaagcatttgttgggtactgggctgtgttcaagacactttgctgtgttttgtattaccaaaatgggtatggtccccaaactcttcagagcttacaatttttaggatgtgtatttggaaaattaatagaagaattagtaagatattttttctattattgttgcatagggaaacatttcatttgccatcctggatttctgaaaaatcaaatggaataaatatagactaagactgcttggcagatatacttcatgtcttagtaaaacaagaaagtgggcaaataaggctaatacaaaataatgtttgcatcagagtgaaaattataccatgaacaagtttcttcaaattctgaaatagtctatttattattattttatctcataagttcaaggcagtttttataggaagcttatttattttctaagctagcattaaatgattattttgagtttgtggtgaccaaagacaacaacgacaacagcaaaaccaaaaagtagtggcagataaacttgtttccaatcacaaactatgcatttttggaattttttctatattgtaatgaaacagcaagactgctaatatgatttattttaaatatcagaattgtgagatgtggaagaaactaaagttttaaaaaagatttattatatgagagtatgttcatcaacaatgtaatcagaactatccatctgttttgtttggagctcagaagactaaaagacaatgagtatgatttaccttcttccacagttctcttagggtaaaagtttgtttagaaatttgtcatgtgtgcttttattccttgcttacttgcaagacagaagagaatccatatttagaaactagtcagagtggtgtgtgctgtattgagcaactgatattttgaagctctaagcatttttaaggaatgaactcaaccaaaacatgtgattataaaatagcaattgttcttagttgaagtattaccccttagagaaaaatatatttggctggggtgtttggaagccatagaaaaaataaaaatcacacatctgaatattaaaaggtgaaaaggaaacaagacaagaggatgagaaacacctttccagttgtttttgctgcttatgttatgcagttggatgtctaattactaaagcttcattttgttagacctttaaaactaatacttttcatatattttctcctgatttaaacttgaaatctttttgtattggggatgaatcctttagtgtttaaagctgctcaatgagtcaggaaaaaaaaaaaaaaccttgcttttttagactctactacccttgccaatattttctgacatgctgtgggtcatggaaattggcagtttctatctgttaaggacacacctggagcacttgtaaattatttgtgatgatcggttgccagtgatgttatagatccaaaatcagtgtcaaaaaattcttgctgaggtgaatatttagaatttttaatagatttgtaaaaattttggagaactttaggtagcacatgtgaatgtatatatgaagaaataatctattttggggaggaaaaagccttattatttagagttgatcctaaaaaatgttattacagaggaaaccatttaatgggagttaataccccttttctctttttatatattctacctaaaataagactttaaaaagaagataacacataacttaagtttgtttctcttcatgttgtttttgtttttgtttagttttgcatctggtccaatatgccttataggtactattaattaccaaaatttgaatttatccaaataatttttttcactgtgtgtaactgaaaatatgctttgattaattcaaataacttctcagtaagctctgtatctttttacatgcataactcttagtgttatatgctctgttttttatgttatagtcataatggccatgcttcttattaaaaagttttgcatttactttttaattaaatagaataaacattcattgagtagctgctattcataagatattgcgctatatactttttatattagattgaattttgaatatgtggtgttattcattttaaaagtttttatttcattcttctggaatggactgcattctgttttccagatatagtatccttcctgttgccacttagaaattagaaaaaaaatatggattttaaaatatgtctaggcttatattaattgtaggaggtgtttttgttttggtcaagctgggtggttgatcttcaagtcctaaggtctagctcatagatgtttctctcaaattcaacataaacttcaaagacaacataggtcctaaataatattttgattttatctccccatcttaatttattgcatgataaattattatcttggatcgacaccaactacaactctccacaaatcccatattactttaaataccagaagaaaaggccataactatatccattgtgtatgtttttcaggacatgatattgcttttattacctcttatgtgtcctacgtttgttattatagcatgtgtcttttatgtaaacatattcaggaacttacaagatctcaatcaaagtatgattccacatgtatcatgaaacctctaagaaagaagaaattaagtcaaggttctcacaaaactcatgccttctgatgatttctggttatttttgctatggatgtttccttgacaataccagcaatttgtctttaccgtcagttattattcaatagttgtaaaaacttgattgtaaccatataggcaagcaagataggaaacttctttgtagcccgttggattcttggagtatttgtccctctgacttagcgttggttctcaacctccagcaaaccttgccaattctgattctagagtgaatttctgcctcttgtcatgtagaaaggagatttggcctttggtgaatccatgcaacctgaccatattatactagatatagactcgatgcaaattaatttgggacattcctagagtgtccaggcttaaacactgttctgatgcttcattcaagttgggcttcagagaagagacaacagtagaatctactttcatctcctgccaaacatctataatctcaaggaggccagcaggcagggcagtcacagctgactcttacggctggatctggacttgctgctaactgaaaacaaacccagagggatacataaacagctttatcttatcctgcacttctcccttgccaaatagaaaaattccatgaaattagttagcagtgttccctgaactattgagaatattttgcttatctgaactgtataaataatgcatcaaaattttatacgatcagttagaagcaacagaaaatgtcattataatttataaggggttaatgttattatcccatcagcagttttatcccaaaaacttagcaaagggaaaaatacttgggttttgaatttctcatgttaatacatgacagactttttaccacacattttaaaggaggcattcaagacacatcttaagtaggagagaaggtttcagtgagaaaggatagtgactgttattgcagagatcaaatatttaaaactaggcatttaattcactggcacaatatggaatgcagccattttataccaaatatcagaatttagaactcaatatgttttttaaaattccatatttgtataaatttttggggtacaagtgtaattttgttacatgaatattttgcttcaggatgaagtcagagcttttagtatggaataagacacattgtacccactaagtaatttttcatcctccactcccctctctcccccatgcttgtgaactcttttctattgtatatttttagcagactatcagcacaagtcttacagatagtattgtgtataccaatagctattttgcctagacggattcctcctggaagttcatacctttatttctgcctattctgtgccctctgagaacaaccatcggttaaaaaggttttagagaccctgtgattcacaaatataagagggaaagaaactaagaaaaggaaaaacagagaaattggccttataggtagacctgtttttgcgatgttgggatcccattttagattttccctcatgctgcttgcatttcttgctttaaaattgttagactgcctgaatttccatgcacagttatttttcatagcttaacaaacatcactttgagtttacaatattatcaattcacatattcacattcaatataaagtatgaatgagttcatgagataagaataggtacaataaaatggagtattttgggagataaaggaaacctatatagagagcatatttagaaaagaagtaactgtgtgtgtctagcaacctaggtgagacagtgagggggagaggaccaaaggctaacattccatatgaaaaatgtacccaatgtcttctccatgtccagaaaaagtatagagaaagaaagaaaaacacccccaaacttctgcctcacatttctgctcccaactcaggtaaattctaaaattttagaactatgaaacctagatgtcattcacatagttagtaaagaaaatgtgttctaagatcatataacaaatttaatttttataaacggagttttgcagttttctaagtgcctgttgtcttcgaaaaccagagttagggcttgtggtagcttgttgttctagtatggcctcttttgtcttttaggttctgaatgatgctgagaagtaaaaactatgtatttttatctgtgcaaaacaaatttgagatagaaagcattaatacagtaagacaagaaagaaacataataataagagagagggccctaagtatttggctatcttataagttactaattcatagaatgattaaacattttaaatccaaagtcaatggcattaggaattttaaaacaaacatacattctattacaaagatacatgcatgcgtatgtttattgcagcactattcacaatagcaaaggcatggaatcaacccagttgcccatcaatgatagacttgattaacagaatgtagtacatatacaccatggaatactctgcagccataaaaaggaatgagatcatgtcctttgcaaggacatggatggagctggaagccattatcctcagcgaactaatgcaggaacataaaaccaaacactgcatgtcctcacttacaagcgggagctgaattgtgaggtcacatggacacagggtggggaacaagacacactggggcctgttgagggtggggtggggagagagagaacattaggaaaaatagctaatgtgtgccgggcttaatacctaggtgacaggttgatgggtgcagcaaaccaccatggcacatgttgacgtatgtaacaaatctgaacatcctgcacatgtaccccagaacttaaaattaaaattaaaaaaaaattttgagaaaaaaggaaagaagatcctaacaaataaaagcaaaagcttagtgatatactttcgaggataaggaataagccagattttagattgtttttggtagcattacaggagtccagacaaaacaggcagatacatttcactagtttgttaagagttagattttgtaatggttagattatccatcacaaacctggagggtggtggaaggtgctggctagaagaatggggtttggactcaataaaacctggggacaacctagttaagagctaattcttctagggcagttaaaaaaatctttctaagccccagtttcctcatcaataaaacatggataaaaatatttgtttattagacttatgaggattagctgaaaaaaattcataaaaagcacttagcatagttgctggaacataagtgcccaatgaatggttaactattatcatgttagaaattcttttgaattttggactactgatacttgtacttaatttagaaaatttgactttttgttatctatattaatgtttgaggacttaaaactagggattaggtaataaggtatataatttttttatttgtaaatactccttaatactaatattactttttgggtgaacagatgcttcgatagtatactttttcatggtgatatcaggttctttgccaatatgtgtgtgtgtgtgtgtgtatgtgtgtgtgtgtgtgtctatgcatgcgtatatatcccccaaaatatcaaatgatgaaattttgcatttagtgtgtgcatgacttaacataccctagatttattaagcacttgatttatctttaaatttgatctattggtctatggtgctgttagctctctctaatatcagctaaggctatatttagtagaaactattttcttttactattgaaacataatgatgacagcatatattagagggaaagtccttacaattttctctcttgtaccaatgatggagttgcaaagtattaaagataatttgccatttattgtggatgggacacctgatccaatgttcagtcagtaatattatttaatttggcaaaaattaactggagtgtttagaatgtttctcctccttgtcagttgtttagagatgacagttttaatataatagactcattttggtttattctattttttctttcctttcaatttttattttagattcaggaggtacatgtgcaggttgtgcaggtttgtcacctaggtgtattgcatgatactgaggttttgggtccagttgatcccatcacccaggtactgaacatagtacccaatagctggtttttcaaccctcatctctcttccttcctcccctatctagtagtcttcagtgtctactgttcccatctttacgtccatgagtacccaatatttagctcccacttataagtgagaacgtgcagtctttggttttattttcttgcattaattcacttaggataatggtctccagctgcgttcatgttgcctcaaagaacaatatttcattcatttttatggctgtgttttattccatggtttctatgtgccacattttcttcatccagtccactgttgaggagcacctaggttgattccatggctttgctgtgaacagtgctgcgatgaacacatgattgctttttggtggaagaatttattttcttttggatatgtatcctgtaatgggattgctgggtcaaataacagttttaagttctttgagaaatctccaaactgctttccacagtggctaaactcatattaccactagcagtgtataaacattcccttttttccacaacctcaccagcatttgttggttttcgactttttaataatagccattctgattgatgtgagatggtatctcattttggttttaatttgcatttctctagtgattagcaacgttgtgcatttttttgtatgtttgttggccacttgtatgtcttcttttgagaagtgtctgttcacccattttgatatcttgaacatttcaatttgtataattacttggtgagaatagactattaacttgtttttgtaaaactgttcaaaaaaacttatggtgcaaactatttgcattcagacatcaaattattttgatttctagttgttgctttcttaatattggaacatggtaaactctagatggaaaagaacaaatttagaacaaattttattttctaaaaatgcataaattctcttcttactcttctccctcctctctatttttcatttggaaatggttttggtgaagtttgtttcaaattatagatttctggatttccaacattcataatttatggaagaaatagatgcctcatcacataaattaactttatcctgactcatctgtgtcctaatatgattgttggtttgttaataagactttttaatatcctcattaatttatgtgttcaatgtttattccttaagcatttattggttgtaccgaaggcacaaaacaatgtgtaaggccctgaatgtgctgtatagtataattcaatctcatttctttcctcaaaaagtttgttttctagtagaagagttgagttatgcactaataatataatttaaggtccaaacagtttttttttataaataaaacaaactttagagaggagcatatgccatggaaattcaattagataacattagattgtgataccaatgttatgacttgtgctctcactatttgcttttcaatgaatgtgttacctgagtcacttttccattcaattatattatttttagaaaaacttattttattaaaataattatgaatattgaattaatgtaaatgtattgttattagcaaatgtaagctatactgtttactgcaggatatatctaacattagctatccttaatctctgcttaatttaacttgatttctttaaactcagactttaatagaacataaattgttgaatcatcctgaacctcttctttaaaattaaaataccttataggggatatacagggcctggctccaaagatgtagatcaccagcagtcctggagggtgtttgttcttcagccttaaagagatctccttaggagaattgcttatttgatattgctattggaattctcatttatttagaaatgttttgatggattggatcaaattaaaagaccaagtcaacatatgcacaattatcttaaatattatgaaatctgattgacttatggtcagtgctgatagaagagagaaaacaaatataaagttacaaattcactgaagtcattccggatgcatgttttttgacatgatctttagaaaatccttttgtaattttttgatttactttattctgaggaattgcagagaaatggtttctattgccaatagatgtaatctttaagtgcctcataattttcctgagtacttgaaatattttttcagtaatactgataaagctgtggctaaataactaaagagattataaactgaaagatgcaattagtctccttaataagatctgttttaagataaattccttcaggactgatgacagacaaaatagatttgggggttttgtgttttcctttaaccttccattttgatggttcactgttgtcactgttatctcagattggccaaagctatagctataatgtccatttttgttagttggaaccttttcagcttcatattcctttgcaaatcttttctattctatttccttcagagtcttgttatctgaattatgtccaaatttttaaagatgttagtgatttgaaatactacccttccaagagggcacattaacctttaacaagagttcttaattcttgaatccataaaagtgaatgttggttattgtagataaagtagcagaccaagaggaggtatttgggtgcagaaaggtctttgagtgcctatggaatctctttggtcatttctggatttaaatgggcctcatctgcagtttctcagatataaaattttacgaggtttgttttctaatcacaaaatcctcaatcactatgtgaaaagttgagtttattggaaagttatcttaaaaagtacaaattttccctcttttcagagatgtatattcagcaacaaagttccatgatttgacacagttgattttaggggctgagaacttatagttttgctgttattaaagaaagcacagaaagcatgtctatttcctgctacagtccttacttcctattctagttatgaaaaggagatgctagaaactgctgtaaataatctgtcttcaacttctgccaagaaaggaatgtcaagaaaggaattctggagatcatctggaatttgtctgactgataatatagtgcagagcagttctaaaaataaagacaggattgttgtccatgaggagcagcgtatttcatcattgtttttcagttgtagccttgtcccttgtgtggtggtttctgtttctcagaacaagctaagaagagcagttctgttcctgtgttctgggaatgcttgagtatggagtctgtgtttttaaaaagaatattggctcatataaggagtctatgttctgcaattgtaattttttatgcaagtaagagggactagcacccacaggaattaagcctgagaaatgggatagaaatgccactgtccaataaagagaagtgctttttaattttaattttaatttatttatttgtttgagacggaggcttgctctgtcgtccaggctggagtgaagtggcatgaccttggctcattgctgtgttaaagcgattctcctgcctcagcctcccgagtagctgggattacaggcgcgagccaccatacctggctcattttttgtatttttagtagagacaggattcactatgttggccaggctggtcttgaactcctgaccccaagtgatccctctgcctcggcctcccaaacggctgcgattataggcatgagccactgtgcacagtcagaagtgcttttttattgatattattttgaaccaccgcacccagtcagcagtgcttttttattgatattatattggtcttctgaacagaaaacataacagcattactaaacagggggtctgatttgttatcttgtttttgcttgattgggaaagtcaacatagattttatggcagcaggaaattttagttatctattcatacccaaggttatcagactttgagacaatgctcacttttcctgtgcttatctgaatttactttaatcaaataaggatcctacagtacataatcttaaaatataccctgaaatacagtgcagaagtttcctaagaacttggtagctgtacacgctatagttaatagattttctctgagatctattgaagtatggagaagacctcaaggttattactaatcttataatttacttcacattgtaattttagatctatagcttggtaatcttttttttttttcttttttagatactagctcagtcatgtactgaaatcctagaattgcagccctcaagtgtctgtgtggggggtaagtgtttagaatttaaaaataccatgtaagaaattgtatttttttcctagctaaaatatatgatatgaaactcatgttggaaaaagaatagcaaaattaacttttttggcatcttttttgtgtaagaatttttaaataattccttgaaatatttctattttaaattcaaatttttaagattggaatgttattttaaataatgagcatattgtgtgacaggcttttagaatcttctgtatatttagttcacgcttgagacctttcctatggcaaataaaataaaatatgaaattgtttcttttgtctgattatacaaacagtcaggtatatgtagactttgagctccatgctttgatgacctggacaaagtacagaatctgccagctcagggcacataaggaagtgacttatggacggattgcaggaaggaagcaggtggctttatcagctggtgctaagcacatactcaggttttcacacctcccagccatccttgcccagacagagtgtgattctctctggcctattttgtttctttttcaagcctgacctacagttcattttaacctgactttactttaaaaaggaaaaatattgggtctggtgggggagaaaatatgttgtgtaatgtaccatagagcctctcagccagttgtgcatggtctcacattctgcatcttctagattccctaatgaatgaatatggagatgtttaaggaaagtagtgcatctgaacttcccagggaaggattatcttccctaacagtgaaggttaatacatttctagagataacaaagaaaaataaatggaaaaataatattaaaatagtagctgttgtttattaaactccaattttgtgctgagggtatactgggttgtttgcatttataattttacctaatccttataacaacactattgaaagttttatacatccctggtttacagatggggaagctgaggcagagacaggttcagtacttgctactagtaattaccctgtttgaccctttcctgtctgacttgaatgcccatactcttaccgaatcacattttacctgcactttattttaacttgcaagtgaaattcctttcataacatagtaaatgaaaagtgggttggcagtagtttcttgcatagttaatgactatattatacttgtattatgttagatatgagaagttttagaggactggtaatgttaaattaaaatagagaacaggcctgaagaagccttaagcaggcaaaaacacttaggcctcgtaagtagccttaaccttatttgatttacaaatataattggaatttaatttgaactatttattgtcaatgcctacattaagaaaaatagaatttaagtgcaaccaatcaaaaacaaccaacaaacttatataactagggactttccacaggatagaccaaacaaggcaactgtgtaactataacccgtcaaatactttctttggtttacttccacgttggttctataaaagcctcccccttgtattcccttggtaaagctgccaaacctcttctgatttggatctgtctaattcaagaatcttctgctcaaataaactatttaaaattttatttgtgcttcaatttaccttttaaataagcgttaatcactattaaaatagttagtatactatcttgcagcttagcagtaaacatgcattgtctgtcaagatatttgatcagttaactttcattccagcctattctctcactctctaacaagttttagtgcttgcttctactataaataatggtacaataagttaaatcaacttttgtgactggccatcgattattagtaattcaaaatacgtgatcttttgattatttataggtttgaaatagaaaatgaatcaggattagtaaggtgaagaagtggttaaagaagaattctgtatggaacaggcatagattaaagaaagtactacatacgacttttcgaagaactaaaaaacgtacataattagttttatttacattcccaaactttaatacctgagagtagggaaaaacaaagttttttccccctggtattttcctaaggatagaagttgaggaggtttgtatagtatctgtctgggaatgactgttaatgcttctgttctataccttggtcactactgggaatcactttgtttagaaaataaaataatgcatccccatgtggcagtgaatggtatcttcactacatttacctagagggtctgggaaagccagcacagttggaaatatagagctatgtcttcattgctattagttaactgaatagtatgaaaaaaattattctttgcaccttaatactttcatttcagcttgtgttttttatttttttggtaaagcagtgattaaatgtttatttttccttttacttctagaggaatttcagattgtcttaagtggaagaggattcatgctgggcagtcggaatggcagtgttctctgcacttacactgtaaatgaaacatatacaacgagtaggtccccagtaatctcagtgttgtcttaatctctgcaaagcacatcagcatcaaatgttttctttttttctccaacgaaaactaactgacatattttttaatctaagaagaaagaaatgaagtctgaatgataatacaaatagtagtgataattatttaatagttatttaataatggcaacatttgtttataacttaataaatgctggaccctgtcctaacttccctcattgaacagatgatggtttgtggctaaaagaatgaaattacttgcatgtggttacacagctaataatggacagagctgatatttgaacccacttttcctgactgtactctgtacctactacatcaaacacatgatttaattgcttctttcctcaggcttctttcttgtcacacctgccttaaaatctaattatcttcagcttctctcctacttgttcctttttttctttctttttttttgtcacttgttttctttatctaatttcccaataatttgttaatttattgaagatagtctttttttgccttatagatttttgcatctcaaattctcacccagcatagttttggttgataattttcgtatttattgaattgagttttaattttccctacctttgacttttaaaaaagccaagagtacgtttttgttttcattctgaaaaaaccctccctcttaaatagaccttagttttctgagcatcttgagtatctatataaatgtttttccttattaattattatttgcccttcaaaatttatattgtaaagcagattttataactttgtaaactctggtagagttatttttttagattcatctgccattttgctgtttatctgagtataaacactccaattaacacagctttggccatacacacagtacctattcaatatgtgtttattgaattaaaatggtaaggatcttagcatgctaaatgtcataattaattcctctcttttctattaagatcacaattttagtaaggcaaaaatttacaaactctcttcatagtaggttccacaattgagtaggacaagataattttatttttattcagtagtcatttgtatctaagcattagcttcacctagctccttttgattacatatttgagttcattataaaataaacgtatttcatgtttagctacataagattaggtttattaaagtaaatcctcaaataagtgaaattggtattttcctcttttagattctagaatgatcagtttgactaagtaacttttctgcatattttagtctaaatcatttttttcatattttattaaagttacaagggcgatttaagtatgtgacttcataaaatgcatttatttattgtagacaaatgttttattaaacttgtgaaataagttcattttcctttggacatctaactttcaccttccttcatcttttctaaagtgtgcttttaaaataatacggagcatttgtttttaaaaatctgagatatttaaaaaatttaagagaaaatatattttatagtatctagaatggtatgtgaacttagtctaagtcaaaataatcctaacttatgatttaccaacactatatgtcaatggagcatataatctctttccatttttcattccctttcacttctctttctttagtttttttatgttaatatatctagtattccacatttgaactctgattgaagcatgctgtcatattattaatgaaactgtgtgagatgcataaaaccctttactaattcagtggcattttattttctaggtgtaaaaccagtaagtgtacagcttaattctatgctttgtcctgcacctatcctgaataaagctggagagtaagtacttaatttaaaacaattttataacatttttagtaaaaaattattagaaaatggtgacatatacattggtcattttattctatctttttttgatatacagagcattaaaaagtaagccttgggcaagccacttaacttagtactaggcaagcaagttagtacttcagctatactcttagtaggttgaaaatgctaaaaagtgttttagctagtttcttcaatactgattaaaaggctctatgaatatttatctggatgggtttgagaactattattagaatctataaataatcagagccataaacattattttaaacacacacacatgcacacgcacacacatgtgtgtgcatgcacacacccagttcaaaagtgagattttcaataattactatagtatttaataaatattgcgattaaagtgcctcaagaacatggctaaaaaaaggcacaccttgcactaggaaatacttgtacatttgtattaacttgtgctttgtgttccatttctgttaaggtctatcttttcatcttcttttgtgtggccaaaaaaccacgatatctgctaaaaagaaaaataaatttagtttggatgaaggagtcttatttgaattgtttatacacaagtaattctactgataatttagtgttcccatttgtgatttttttcagttctatgatgaagcaaatgattacaaatagttcagatattctctagcccaacagtagagcatgtttaagatagttaataggatacagttttctaggatctctttatatagatctgatactgaaagcagggaaaaattacatatacaaggccacaatccaaaatctggattagtatttagagactgaaaatgatattttcctctatgaaactaagaaaatcattctagcatttttaagctgacttgaacatttatcttttgtggatacttaaatattcaagatatctatcgcacatcacttttggtcatgcaagtaaattggagcagtatcagctttctgttttaaatagattcaaagaagctgcacaagatggcttacaaaactaagagtattattcgttttctgtatcatgtaataatctatgttattgacaacatgcctgtggctaaattaaattggcattggaatttgaggcaagtcagtttcttgtggagccatcttaaccccaagcacaccgtaggactcaaggctttctgaataatcgtgtctgtattagccatgatacatctattgacttaaagtttagtcttgttttagtgaatctgtaattccaggttggtcctcctgaatattcccccaatctatctcccaaactgtattttccattttagttattggcagctccatcatttttgttgttcaggtaaaaatcttttgagtcattattgatttctttctctctcttagacccagtctatcaacaaatcttagctctaatttcaaaatttatccaaaatccaatctctgctttctacctatactgctaccattctagaccaagttactgccatctcttgcttggattattagtctctcaactggtctttctgtttccatccttcacaagagtgatcttgttaaaacagattattttatgtcatctcttcacttaagattctctgtgatgagttctttaccccactcagagtaaagtctaattagtgtaatactgtaccaggttctacagggtctggcctctcattccctctcagacttcatcttctatttgctctttctactcagttatggtcacactggcctctttgctgttcttgaccacacctgcacactcctgcacctgcacttttatacttcttgttgcctctgcttggattgctcttcccctgatgtagaactgtatgtgaacttagtctaagtaaaacaaaacaaatcctaacttaccctatttaatcatgcaatcttcctctttgagtctgtaacctgtatacctttttcatgttttattttttctccaaagccaattgtttgtctgttattatgtttctacttcctgctaggacacgttttgtcttgtttcttgttgtattactagcacttaacacaatgtctgactcatagtagatttactaaatgtttggtggataaaaggatgaatgaatttttcttaaatatttttgtttaccagttaacataacgatctgaaagaggtcatttgattggtgggatcaggtacttgtaccaggatagaggaaaatggtcagaattttattttggggggccatactatttaaagcagaaaaaaaaaatcttggctttcaaataatagtgactagttaaagaaagtaatttactttcttctctgaagtagttataaaatgcattatgcctgttttatcccaggcactgattttgttttctctagaatttgcctttgggcacagtcttctttagaagagaataccatcggggctagtgatacactttaattatttgtagataggttaaatacccaggagatacattttttaatcacacatagcataataaatatcagacatgagaaatcatcattcatttagattttgacattaatatttcacctacaagcaaactgagaatgttctgggacagagatgtgtctaaaagaattgcttttgtcttcctttcctggatcaggtacatgataccattgaagcaaagctttagattttatttgttaatgtactgggtatttggggacatcattatttattttacttagtatgttgcaagtaatttggacctttcctgtgccttgaattttcttcagacacttttgtaggtgatgagctctcttatgagattggattgaaatcaacacaattgaaacagtttagccttgtaaggtgactgatgcttaatatgtttgcctaattagcttttggctaaggattcaaacccattaagtagctagttagtctatgtttcagataaacacatctggcccaacaatgaatgaaggtttttcctgtgtgagggtgacagatgtggtgatagttcccctcattctctcacttaaataattaacaacatgctggtgaagtaaccctagacagagagcctcaaagccagatgcacagtctcagagctgcatccatgggcatgtgcctcatctcattagcctgctctatccgttcttttcttatctttatgttatttcatgaatttcactttgtttgaggattattcttttaaaaacttaaggcctgtggattttggtttaaaagagcatgtgggagatgggggagtaagaatgtagccgagtagctaggacataggtcgtcatttggaatttgatgttatggagtaaaggaaagttaaagttatctgggcaaatcctgtccaggtatatcattttgagcattgttcagatgggtctttcaataactttttttgatagatctgtctgatacagagatctgtatttattaaatttctttatttgatatagatttttaggctaagatgaacaaactcttattatttaaacagaaagaatggtcatagtagtgaggttgttttctatttttttggatgaacacggtttacttttaaaatggtgtatgattaaacaaactgttttaccaagaaaaatctgcctattatttagaaatttatttctaaaagtggttcctttgtgccaaattgtcttctttaagtttgaataggatgcattctctgatgttctttggatgttccctaatccagggcactagcctcacagtttaatgtgcaagtattacaagtatttgcaacgtgggcttattctatgacccttgttttgtgagacatcaacaatagcacagctgttctgcaatagttgagatctcaaacctgtaatttggcaagaagaacagagcaaattgggtaaatatacataaacacattgtgaagaaagaaaaacagaacagaatgaaccggagtcttatcctacagtttttttgatagagatgatttaaggaggcatatggtatagttaaatgtgctttcaaaaacattggccagaatcttctggcaaatatgaaataaatatctaaatcagatgaaactgatagtattagttctcttaaaaggcagtgtgggaagacacttgacagagtgttttgagaaagaagtggtgggacgtcactgtctgtggaaaatttcaaggaaaactgaaggatcattgagatgaacagtgtggcagaattgaggttagagcctggcttcataatagaatctttttattttcttcaactttttttttcagtattaagcctaatctatttttcctggaataaaattgattaattctatttctctttcttgattagataaattctgcctttactacagtttctgttctttccaaaagtatacttctatctgtatgtgtgtgtatgtctatagaatccctaattctcttatgttagtagcaggtgatacaaccacaacattattccttttgcttcttgtgaggaatgagattttattttttggaaaaatcatggatattaagacattagttatgtctttatttgaatatgaacattagaatcatgatgatatgggttaaagctaagggctttctctgtttctctaccccacacttccttggtggaacatttctcttttccttcacatggtgcactgttttcaaattatatctatcaatgtgataagttcacctcatctgcattgcctcttcatcctaagtattatatacggttctggcaatgtcttttaacacatgaacacctggaggggtggctaataagatgtcaccatgtatgggaatgttaaataggatacaatttaaaatactgaaaaaaatactattcagatgtgatgcattaactctccaaacattggaagatctgtcacatgacagacagatttgaattgttctttattgctataggagaaaaagggtgaagttaaggggaagcacatttcagatcaatataaggaaatactttctaaaatttagaattatttagtagataattctactaaattatttagtagataattatttggacaatgaaaagtatttaaagatggacacctatatgaggatgtagagaatattcttgaaataggtacttaaatcctcttatatatctgcttctaaattttatattatcaaagcttttcattattactaaatgtattagtccgttctcacactgctataaagaactgccgagactgggtaatttatcaaggaaagaagtttaattgactcacagttttacatgactggggaagcctcaggaaacttagtcatggcagaaggctaagggggagcaaagaccttcacatggtggcaggagagagaagtgcaagcccaggaaaaatggccacttttaaaaccatcagatctcgtgagattcactcaccatcatgagaacaggatgggggaaaccatccccataacccattcacctcttatcaggatcctccctttacacggggggattacaattcaagaatgagatttgggtggagacacacagcaaaaccatatcactaagactctttaaagttttggatgaaaggcactcaatttgattgatttaggcactaaattgacttggtgattgttttttaagtgtctttttagaatgttattaaaaaacacacagcaaatggaaagtgcttatttttagtgcaatactcagaacaattataatacagctttcctgtgatgtccagtaaatctctgaatatagctctttttccttctgaaatgtagacatcttgctaactgcaatttttgttttatctattacacatattgagatatgctaaaaataagtcatttaattcaaaaatcttttagccacttttaagaaagtatccttagtcaaaacattaatttacatataaactaattactagaagtggtacctttaaactattcatgaatatggaatcttatcttccaacttaaaatctttatcattcataaaactatcttttaaaaagcactgcagaacattaaatagtatcacttgatttcaaaatatcttaagtcaccataattgtaataactttttgagtagctgccagtttaattttcagtatgtgcaaattgaaggtaaattatttattacttccagcaaatgtctttcacaattttatttaattttttcatcctttttaatggatcataaaatgacaatcttttattggatatatataaagtcagaagcaaatgaatactttgcaaatatgttactgtggattgaaatgtcaatcataattcatggagtataacatttcttagatcagaatagctatgacatatggcactgagccattctcctgttttggaagagtcatgcatctttagacattaaatagaagtatgtgactattaggacttagtgagttttatttactaaattgtgtatcatagtactatttaataatttctcatgtggaatgactttactatgtttgaatttaattttattactattaattgtcagttgacatgaaagtacacttcaatcaatctttttgttacattaagttgttaagctttcttctatccttctactttgaaaaaacatattttaatgatggagcaaagcatggattaagatatttgttaagaaatattaaacagtgtagaaccaatttatagaaaattaagttgaaagtgttgccatttcccagcattatcttcccttacctccctgtattatctgcacaaggtgccactcttgtctcagaaggttttcccttgtcttttgtgacttcatattctcaccctagctgtttctcccctttctcctcagatatccctttagactacttttgtttttacagtctcttaactgtcagttttttctttttgaggtggggtctcactgtcacccaggctggagtgaagtggcatgatctgactcactgtacttttggcctttctggctcaggtgatcctcctacttcagcctcctgagtagctggggctacaggcatgtaccaccacaccctgctaattttctgtacattttgtatagacagggtttcaccatgtttcccaggctggtctcaaactcctgggctcaagccaaccacccgccttggcctcccaaagtgttgggattacaggcatgagccacagtgtccagtgcccctcagtttttatcatggttggaggatacttgacttataaactcaccctttctatggctccctttttcttcctgatgagctccaaatttcctggcaagatctttttactagctttaagtctgctgtccaaatgcctcctatccaaatgtctacaaagtcacttggattttctacaggcaccttaaacttggaggtcccaaattaaactcacatccttctgttcaaacttgctccttctgtgttccctatgcaagactgcccacctacttgtgtaagacagaaactttgaggtcatccttttttcttcttcctcacctcctacatccaataaatgtccatgtcctttttattctaactcctaatatgtctcagtttttgttttcacactaacctcattgccactattttagttcaattctactttggtacttatttcaagtacactgagactttcttaactggtctttctacttttgaaatttattcttcagacccctgccttacactgtaacaagaatgatatttgtaaaacacatttctaactatatttctatcaaacttaaaactcttaagacgaattccagatgtctaaagatggcatccagggactcaatgatcttttctctgctccccttttcaacctggagccatttcattcttactctcaccctcttttgttgctggagtactgaatcacttttactgccttgactgttctgtccctgtccctttcttgccttccaccctgtcggagtgctccatctgcatggactcctttctaatctcatttcctaacttctgatcattctttaagcttcatcctgtagtcgacttcctgcaggaaaactttgctaatgcctctatgccaaggccaagtctgttgaatctcccacacagccctctagaacccctgtgcctaaacctatttaatatcgcattcttttgtaattgtttacggggcttccttttatttacattgtaatttgtattatttgttactgcagccattttaccaagtacagcccaattatcattgctactacaatcaattaataaatactcatttaattaactagtaataaaataagtgactgattgagtgattttattggcagatattcctaaaccaatttggagtgtgattttaggcctgaggtttaagcacagagctcaacatagctataatgttggatttaatcaaggtcatgttttataacaggaagaatatggcattgaaaaaattttttacatgaaactgcctgacattatatagtaggcattttttgaacaatttagcttacaatatttttatatggcagtgagcagaatagaaattaattaaattaaacaaaaggtattacataatgattttattgcttctatatacatttcttcttagcttggatctaaaaggtttattaaaaagtaccttgatattctaaataatccattagacaaatattcaggtgttaacggtaatttatggagtacctgttgtgtcccaggtattgtgttgtatattttatgtatattattttaccaagctctgttccttaaaacaactttgaaataggtacagttatccccattttacagattgagaaatggaagattaaaaatggagtgatgagggttcaaactcaggactccagtttcgcccaaatgccctttttcaatattattagatccttggggcaaataaaaatgaactgctctcttgcttcccatatatgtttgcaactaagtaaacacaatgttgttttaatacatttttgcccataagaaagcctctgttactgagcgtatggcttccactgtcagaaacatactaactagaagtagaagttatgtaggagtaggaataacaatccatagatgctaacagaaatgacataaaatatgacagaaaacactaaatatgtatagttgactcttgaggcaaggaaagcttaccgaatttggtttgcattacagtgggcggttttctcaacctatgccagaaaaggagtagcatagctttgggcaaaggctctagcttaatgatggtgccagtaccataccaataatttcaaaaatgtttttctaaagaagcagcaaagtttaaaagcagatgtaactgtgtttttcatatttgcaacttttctgaaaaattttcacagaatacatatatctatacacacatattcataaatatatatattcacaagtaaatatattgctttagcatatagtaaatgccattctagttaaccatgagaaatatgtgaatgtagtaaaaagctagaagattcaaatgaaactcatactaatgagagaaatttaaatataagatgctaaaatacatgtggaggattaaagtagtatgttgtagaagtgaaagcattttgagtacagagtcagtgaagtccaatatttggacaagttacctttataagcttgtttcttcatgggcaaaatggagatgatgctagtgctgacctcagtaggttgttgtgagaattaaatgagaagatgaattaatgtgtctggttcttagtaagaaatcaacaattgcctgtaattatgagtggtgtttatggttacattttgatcacgctgctactactactctcaaaaggaggaagatattcttgtttcagttccatacatcatatatcatcaagtggatttctatatctgcggagaattaactcagtttttgttttttttttacaccagccacattatactagaatgagattttcggagaatgcagttatttttgtcggttacaagatcatatcagcatgcatttctaccataggaaagagtaaactttgttttaaaatgacatcttctgttctcatttcatttttttaagcattaaaagtttgaatgtttccttcattaattagagaacttgttcgtttctggtgaattttaactgtcagattataaacaacttcagtcaatgtctactgatcccttaaagttgaatcagtaagattacctgcaagaatcacatattattatgaatgaaaattatctttccaaaaaataaaaccatgccaacatgtcagatgattcaggccacattctttaaagtagaggaattaagaactagggaagacaattttttatttgattatgcttagtcattaaaaaaacaaaacaaggcacccatttcttcagaatgagcaattaggtttaagttttctttcttaatacgccacttggccttctgtgttggaatagttcagaagtatagttaattagatgtaaacatccggaattcttattctcttctggatttagctaaaccatctttttttttttttttttttttttgagatggagtctcgctctgtcgcccaggctgggggtgcagtggtgtgatctcggctcactgcaacctctgcctcccgggttcaagtgattctccagcctcagcctcccaagtagctgggattacaggcacgtgccaccatgcccggctaatttttttgtatttttagtagagacggggtttcaccgtgttagccaggatggtctcaatctcctgacctcgtgatctacccaaagtgctgggattacaggcgtgagccactgcgcccagcctagctaaaccatctttttaaacaagatgctttgaagtaaatactttacacagaaatattttaatagaagtatttctatattttgattgaatttagcttgaactttgccaggaatatccttcccagtagctttcttgtatgaatccaaaccaaatagcacattgagaaaaaaaacacattaaggactttgatttcatcatttaatcaagcctctctgatatgatggaaagagttcaaagttagaatcctagctgtattactttgggcaaagtaattaacatttctgggcctcctgccccccatctgtaaaactgtgaaactactatttacttcaagggctattgtgaagggttgaaatagataatgcccataaaatgcctaggatagcatctggcaatagtaagaactgaataatatttcttatctcccctttctgtcaacagcttgagattgtttctgggagtttgttttttgtttttgtttttgcttttttttttgacagagtctcactgtgttgctgaagctggagtgcagtggctcaatcagagatgactgcagcctcaacctcccaggctcaagcgatcctcctgccacagcctcctgaatagttggacctataggcacatgccaccatgcccggctaattatttttatttttactttttgtagagatgggcatctcactgtgttgccggggctggtctcaaactcctgggttcatgcagtcctcccacctcagccttccaaagggctgggactataggcgtgagccaccatgcctaccctgtggatggatggctttgatttgtttgcaaaccaattatatgataatatcctttgtaaactattttggaaaagagctctttccatacaaaccctcttgtcatttttttcccttttgatatcttgttttagttatttttataataaaaaattactaactctaggctaatcccaaataatcaatctaaattaatgaaattttaatgattattacttgttttataatttgaaatcatattttcaaaataagaactatattttaactataatatttcttagccagggacaaaaattattatctcttctttgtacctttgtcgggatattttaatatctttggaccatctgcaacaaatacctgcaaataaatctttctccctttcttttctttgttgctgtctttcatattcactttctttatattttatttttgactggtttattgcttccagttttatattcttagtagtaatacctagagacagagaaataggctaaatgggatctaaagcttttctttctttgtaggtttttgtgaacctgaaacaaccttggcatttttctggatttagaaatttcttaatgcctgtatttctgttactactttgtaaagaaacaattagtccacttaataagtttgcctgactaataagtaaagactagaactaaggaagaaaaaagctggcataatttaccaaaagctgtgactcttactctttccttttttttgagactgagtctcgctgttgtcggcccgggctggagtgcaatggcatgatcttggctcactgcaacctccgcctcccaggttccagcaattctcctgcctcagcctcctgagtagctgagattacaggggcccaccaccacacctggctaatttttgtattttcactagagatggggtttcactatgttggccatgctgggcacggtggctcatgcctgtaatcctaggactttgggaggccgaggtgggtggatcatctgaggtcaggagtttgagacccttactctttaagtatgttttctggctggttttgaattacataaggaaataaaaatctcctactcctctggttttaaatgtatataaaattattgtttcttttctttttcagaactcttgatgtttcagtgagctttaatggaggaaaatctgtcatttcaggatcattaattgtcacagccacagaatgtgtaagtaaaagtttgcacaaagattatcttttaaaagcaattccatccagaaataatctatggatctttggtgttgttgcatactacaataaaggagatgcaaacaataacattggagaccattacaaggaaaaattttgtatagaattcgttttgggtggcattgcttttttttatgttgagctcccttttataggaactttaaggtagtatgcatttgattttcatacttatagaatttaaaaggcatatttgaaatcaaaataaggtaagataataatttacaaatgaagcactttttttcttaaaacattaaatatagaaattatgttttctgttaggaaggttagattacagtatctaaacatttatcatggtttgaaatcaaattattgcattttaatgtgtgacttttttattttgttcatttaaaatattggtgctttctgaatgaggaagttgactttgacagcttgatactatcttaatttgtaaccacatcgaatatatattttgagattactgatttgcattacctaaatgcagtattttgaaatataagctccattcagtagacattttcttccttttatttaattttgtggatttatagaaagtttcaatattatattattgcacgtggaaacgggcagcactaaacaggaccaaccacatggttattatttctaaatatattttgtaaaaaatatgtaaatcaaagcaaaaaaagaaaaattcctgccctccaaaagaccaaaatcctaaacctaaacattttctcacatctcacggtttcattgttttatattttgattttgatctactaattcgagagatacccttccaacagaaaacaaaacaaaacaattcaatgttatctgtatagaggtagttgcatttctcattttaagacaatgcttcaggtaaaaggcacatcatatggacccttgggtcactttttaactccttggataagtctttaccaacagctacttctattttgaaaatagctaggttttttgtttttgtatttgtgctttttggggagggttttttttctcccatttctagcgcatgttgcaatttgtagattttgtggatgttgcagtttgctggttttctggtccctcaggatgtggttgaaattacaaatatttgtggtttgtagtttctcagtgtttagctcactgaaatggcattccttcctcatccttcttcctattctacacctacaaactgtctttttcttcctttcttccatgcaagcaggaaaacaggtctttgttcctatcctataacaatgcatgattttcacatgcaaggatgctgaggctaaatgactcatgtgggatgtataggttggtgctttaaggaagactttctagttcttgctcctgagtgagggtaatatgactcaagatggtttaagagtggggagatttcagaaaaatgatcattgatgtgtcaatgatgaggtaaggggaaaggagcaagctgtaacagtcaagaaggggtcaagatgattctccgtaagaaggaaggctttgagaaaagacttgaaggtaagggagtgaggcctgcagaccatctgagggaatagtgctccacacagagggagcagctagagcaaagcctgaggtgacaagtctctggcaagtttcaagaagagcaaccagccagtgtgctggaatggaagtagtcagggagagtgggagcctctggataaggaagagagagtggcaggagcagggcagatcctgtagggtgtatagttattgtcaggacctgagcttttcttctgagtgaaacaggaagaccttggaaggtgacatgatttgattcactgttttgaaaggatcactctggctgctgtatgaaaacagactttgcgagtagcagcaaacgtggaaacatcaagcccaattctgtggctattgcagtaatgcaggaatccttccccacccagaggagggtagccccactggggtgatcaaattggagatggtgagaagggttgaattagaatatattttacaggggaagtttatgggatgcctagacagattaggtgtgggatgtgagataaagaaagaaattaagaatagctccaagtgtttttgcctgagcaactattaggttgatgcaaaagcaactgtggttttgccaaatagaaggatgaattgtcatcaggcacagcagtttcagagcaaagagaagttcagttgtggtgaggttgaatgagggatgtctattggacatcgcagtggaaatgtcaggcccctgaatatgcaagtctggagttctgagagaggtctggattggatgtacaaactttggagctgttagttgttgacaaatggatggaataaattaggcaacaaaatttcacataatatttactgggaaatagtggcttccaggagcttagagcaagaagggaaagagtaccagaaaaaggttttatgcttgtggaggaactgagacagacagggccttgaagggagactaggatgtcagcaaagaaaggggatggggagcatattatagagaaagagcaaacagggtggggatgtgttgacgaaattgagaggagatgcatttgactggagtagaaataagtggaaggaattcttggaaatagaaaatgtagaccagattatcaacaacctaagaaaccgaattgaatttagagttcatcctacaggctgtgattcccaaactctacggtgttgaaatgaaaaaaggaaagaaaatggattatatttttcatattattagatttattcaacttgaaagaattcttttattttaaaattgttctttgtcctattttaattttaaaatatcctttgattgaggaaatacacttcatttgtcaggaccgacctctccctcctttcctttcctttcctttcctttcctttcctttcctttcctttcctttcctttcctttcctttcctttcctttcctttttcctttcctttcctttcctttcctttcctttcctttcctttcctttcctttcctttcctttcctttcctgtcctctccttccttccttccctccctccctccctcattccatccttccttctttcttctctcccaccttccttctccctagtccttcagtcctttcttttccttcctcatattcctctccctcctcctgcttcttctcttcttttctctttttccttgtatgctgttacttggcagaattattagctggcaagtttatgtgagcagattttttttttcaggttttattgccttgagaaatcacaaggtctgactccaataaataataatatggttttaaagagctttggacacacatgtgatggtgaaacatactattttgggaagattaaattagtaatgacatgtaagagaaaggacaggaaaagaaaaggcacagagaaagcttttaggaaacaaatgctagaatataggcataaaactttagtttctaaattgactgatggtggtagaaatagtcctagaaatagccatataaattgtattaatgaagaaatccatattataaagtgacttactggtggttatgggggaatgtttcattccaaaatattgaatctcatgggctggaagaatgatgctgccattggaaacatccaaagcgagaggtaaatctggagatcagaagaattttcattgtagacatgttgtgtaagtgaggttggcaatgtcaagcaggtacttgtagctatagcttcacatctcgtgagtgatcagggctggaagtaaaaatttaagttttgcttttgctgaggttgtaggaaggggaagaaaaatgggacaatttagagttaatgtcattttgaagtggggaggaatacatagagtcaaagacaaaaatgcactggacaaaattaaacaggaaaagaagaccttgtcaagactgttgcaagagggaagagtgaatttacctgtgatgaaacaaaaggttggagaattttcaaagctagggtggtgggaattataggccaccagtatctgctaattgaccttacctaaaggaaaagtaaactttctcctactttcctaataggaggtagtgctaaaacttggagcaaggcaccactgatgttaggtttctccgcttctacagagaccgtgtgataggggcactatctcccttgatgattacatctcaaaggaatggctcccagggccttgagaaagacagtcctccaaggtctgtaaaactgctgagagttaaaggagatttaaatgcttttcaaaaggcggagaaatattttgtgattacaagtttttatttatttatttatatatatatattttttgagactgagtcttgttctgttggtaggctggagtgcagtgacgccagtttggctcactgcaatctctgcttcccgggttcaagcaattctcatgcctcagcttcccaagtagctgggattacaggtgtgtgccgccacacccagctaattttttatttttagtagagacggggtttcaccacgttggccaggatggtctcaatctcctgaccttgtgatttggccgcctcagtctcccaaagtgctgggattacaggcgtgagccacctcacccggccacaaatttttaaaagtagattcttaaagaaaagggagatccaagacccacagttagaagaaatccaatttgactgaagtttaaagtttaatcaagcagaagaaataggtaaggctgtcttgatgaataaagtaagtgaagaaataagagaaagaactgcttaagacgtgggaagaccagacaccgagtgaagaagacaaaggaagatagaattccaaggtgaagagagggcagcagtacatgcctgggagaggccaaggtgagccagggctgagaaaatgtccgaggggtcaccggtgatctccccgtgtctgccaaggctgatgacttgaagccttctctttcttatgacttttattattccactagttattacaggataataaatactacaattcaatttttcaggttgacagtaattataggaaaattatttttcctccttaaatttaaccattgccttgtatttaacctcaaattcctgttgcctgtggtagtgatattaccattccatagatatgaaaagtgagacttgcgctgatgagatgcacctgctgatttatgtatgaaatatctattgaaaatagaagagtatagtgggtaatactatgatgaatacctggaccttggatttaaatgatatttagtctttcaggcatatttgcatatggtgaacaggtgaaatgcagttagcgatattgctcattgtgaggataatgctttttacttgctcaggcatttgaaagccaaacaaagtagaacactattattgtagtcattggttagtccttgttaaaatctcacaaataaagaagctcctctgtatgcttcaggaacatccagggctcagtcctattgaggccttaggaactgagctgtgagttcctgtttgcttctgagcctttcttaccagccttgagacattgacggcagggcttgtgctgtgcttattgcccgtctctcacaactggtccttacacagagcaagtgctccaaaattatttcctaaatgaatgattgaaagtaaccatatatctagtttaaaatggggaaactaagttttagatttaagagatttaagattagagtctgaggagggtttgtatttagacaatcctttatgttcatctctcctcaaaagatctttaaaatggatctctgcttttaaaaacataatggaaagatgtccttttttttctcttggaatcactggtcttctgactcttttgcaataacaataccaagaacctgtttgctgagcaagtattaagtgctagacgctgtgttcagctctgctgtatatgtgctaaatcctttatggcttcatttagcactcacaatccaagaggaggctgttcagattatgttaattttgcagatgagaaaactgaggaacaggaagatacctccaacattcagtgcacttttatccaaatacattagaaggtacttcttcttatgtggacccagccctcaaagaggtgcatggtttgatgagtgtaacagcaggtggtttcagtccttactgtccctgcaaaagggatacttgaagcctggaggctctgaaaggttgttacttgcccagttttgcagtctgataaatgctataattcagaattgaacccacgtctgtctcccccaccccctgactgaacacttccacaagttgccattttaaaccagattctacattgatttatataggacaagggttggcaaatttttcctgtagaaggccaaatagtaaatacatacttaaggctttgggggccatatggtctctgtcacaactacttaactctaccattgtagtgcaaaaacagctatagacattatataaacaaatggctgtgttcagtaaaactatgtacactgacattggaatttcttgtaacatattcttcttttgattttttcagctttatgtaatactgtaaaaaaaaaaaaggtattagcttgctggggtcttaccaaatagattggctggatttgactcttgtaccatagtttcctaattcctgatatcaaatattccaaaaagaatccagtaaatgaaataattatctgtttcatgactctgttttctttccctaactctaaattcatgaaacaaaattgtggagacccattttccccgttctgtgtctgtttcattctaatgtggctgctgtgaattcactattttctagatagattgcagcaataataactaatgctcttttgaaactttccagtgattcctctgtcccatagttatgttgcaagggcagcagtcaccctgatttgtttttatatctctatgattctcttcactagaattttagttatatttggcagaataaagttttttattctataacttgcttttagtgaagaaaaggaaattagatgcagatgacacagtccctgaatacactaatcccagacatcagctcggttaactggatacaaattcaggctttcccattgtgtttgttttgatagccagagccactatcagagcaagaaagatgttagtcatgaaagaaaaacagtttcattttgcccaaaactttataaacatattggggaaatgtggaacactacagaattcagtcccgtaatcagataccatgttgcaaactatggacttaagagtcagatgaagaagagtatgaaactagatttgattacatatagatctatatcacgagcgaaactagaagcttctcagttttacataggcatgcctctctttagaactgaaaaagttctaataaatatttattactcaaatgtcatttttcttaaacagggtagaaatgttcttggcttgccagggtcttacaaaatagattggctggatttgacctttgtgccatagtttcctaattcctgatattgaatatttcaaaaagaatccagtaaatgaaatagtcatcagatagatagatgatagacagacagatagatagatagatagatgatagacagatagatagatagatagatagatagatagatagatagatagatagcaagaccaggttgcattttccctgacgcttttcccatcatgtttggcttatatatatactaggtctgtaggtctttagaaagtgcataatttatttttaattaatttatttaatttattttttttgagatggagtctttctctgtcgcccaggctggagtgcgtggcatgatcttggctcactgcaacctccgcctcccagattcacgccattctcctgcctcagcctcccgagtagctgggactacaggcgcccaccaccatgcctggctgattttttgtatttttagtagagatggggtttcaccatgttagccaggatggtctcgatctcctgaccttgtgatccgcccacctcaacctcccaaagtgctgcgattacaggtgtgagccactgtgcccggccatttttaatttttttaagacaaatctttgcaatcagtcgatatgataatattgtaataataaattatatttgtatctggagatgcataatattttgtcgatgctactaatggaggctttttttaagggaaaaataaaaaattttgagtatctagcagggctagcccatctactttgtgttctgtcagtgcaattttctgattttcctgttcttggcacatgcatagaagaaacaaactaaccactaaatttttagcagtttcatggcctctagttgcatgtccttatttctgaacatgggtgtgaattctaagaaaaggggaaaccaaatgttcatagagcccttttgaaagctgtatagcacagccaaatggtgtgccatttctatgagtgctgtggctggatcctcatgtgggaaaggtctggtgagaaatgtattccttctgtacatggacagtcacagataggggctgtcttacataagaccatatttttgaaggtgagatcaagatttatacatttgacaatatttctcccatagggtaccacagttttccatgcgtttctatgacatcctttttccgttatattcttgtttgcctagattttagagcttaatgtgtttgtgcctgtggacggtagctgatttattcatgaatatttagaattttgtaaagaaagatgcactttggaatgttctttctaagaacaggctacttaggtatttgcctgaaaatttctataaagtattagtattaatcttctttctcttttgctctttttcttttcttccctgaattttttctcataaaaaaaaaaaccacaaaacaaaacattattactgaggatcctagggattaggcatttgggagataccccttgttcccttggaaactgtttgtacagtaaaagttcagtaaacattcttctttgtatccatgtcttttgctgttttttttctttagagttcattctttcagtaagtagttttttctttctttctttcttttttttttttttgagatggagtctcactctgtctcccaggctggaatgcagtggtgcaatctcggctcatggcaaactccacctcccgggttcaagcaattctcctgcctcagcctcctgagtagctgggattacaggtgcccgccaccacgcccagctaatttttgtatttttagtagagatggggtttcactatgttggccaggctggtctcatactcctgacctcgtgatcacctgcctcggcctcccaaagtgctgggattacaggcgtgagccattgtgcccggcctcagtaagtagtttttaaatgccagacatggagtatacggtgctaagtaggctagatgtaacccctgacttcctagagctcacaataaatcatggtagaaaatataaattaaacaaataatcacaaaattcataattcgttgcatcgtgataagtactaaagattaaagtatagagaaatatgagttgtttaatcaaaggggcattttatagtcaaagggtccaaggaagacttccatgaagtgacttaatatgagccctgtaaaatgagtaggtggtttttatatgaaagggtttaagaacagcgcccagagtaggtgccttaaggctggaggagctcagtagttaggaagggaaagaacaccactgtggtttttgtgtaggtcatatttcagcttcaatgagagaacatctgatttatgaatgcatgagattgcattcttgcattgatgtgtatataccatttcccaacaagaaccattgttggtttgtcatctacttgataattttttttaatattcatcttattatttaactacatgcaataaattgtgtttcaccatcaaaaagtctgcaggattctgcctaaattctgcctatgggtaatttgctttgaaccaaaataatccttacatgaaagatagagaccagtctaatcctgattagtgatttttcctaaattccttttagatgtatttgtaaagaatctgcagcattttgggaaagtttttgtttattatttcctgtcacaacaatataacataaaacataatatatattaaatagaagatacatttgtagaatttcaagaaattaaaataatatagttaaaatctagtttgacattatactttctgaattattttctggtgtttccttaaaatatatatacatttggaatttgaccataagctgtgctgactgctttgtttttgctttttgcctactttagtctaacgggatcgcagccatcattgttattttggtgttactgctactcctggggatcggtttgatgtggtggttttggcccctttgctgcaaagtggtgagtaagaaggatttacaactctacttaaaaaaaattagaaagatgtaaagtaaggtctgaaatatgaataaatgccatgttaataacagcaaagtttcagtttgcaaaatgaattttaattgtcttcactactataataaattctagaaaacatgcaggtcagtaaatatttatttaaaaaattcatctaaaaagcacttttaaatcaggtttgtgggaagaggaggagagaaaaataggggaagtagttgggataaagggtattttcattcaagtgcaagagaaattcaagtaaattgaatgatgttttcttttgggttcattgagaggagttgataatttgtcagtttttttaagtgctcatttaattattcatttcctctttgaagggtctaccatttcatgaccttctctgtttgctactgaaaaatatttgttcggaaattagtcatgcacaattgtcagcatatctgttaaactgctacttttggagtaatgacgaggagtttcagtttgtgtcattttttgtgtttgtataaagcctgtgaaagaatatgggtgtatatcagttatctattgctgctgtaacaaattgccacacatatgttgactaaaagcaacacaaatttattatctaacagctgtctaatcacagctttcacatgagtctcactgggtttacgtggactaaaatgtgttggaagggatgtgtttctttctgaatgctctaaggaagaagccattcctttgccttttccagcttctaaaggctactcagattctttgcctcatacccccctttctctatctgcaaagccagatgtgttgcatcttttcaaccatttttctgtagtcacatctccctctaactctgaacccagctgggaaaggtcctccagttttaaggaaccatgggattagattggacgcacttagctaatccaggatcatctcccatctcaagatcctgaaccttaatcacttctgcaaaatcccttttgccattaagataacatattcacagtaccaggtaattaagttatggagatgttggtggggggtatcattctacacatttcaagcaatccaatttagattgttcaggtttcatagaaaaaaaattatcttaaggaagccctcttccaaatcatgcctactatttattttgagaatattccttttattataagcacattgtgtagaacttaatattgtgtagttcatggtaatgttttacataatactgcaagactgccttttcccgccgctctcatgtttgctgactgatttcatcatccttgcaatttcagatgtgctttgttttagccctgcgtttattattagttgtacttcagtttgctttcatgaggcaacttgacaatcctttatctcagagcagttggcccatggaggaggcatagaattaactcagataacagtagagacaagacgcatcctgtctctttgctgaactaatcagtttcctgccatagcaatttatgctcagtttcctgttgtaatcttgattatctagttatggctattttagggtggctagcattcttgagtatcacattaaaaagcaaaaagatgccctaatgtaatttgtgctccttggttttttaatttaaactttgcagtggtgcatttcccctcctttttttaaattatcactttgcaatttcatttagtattgaccagagggctcagaagtgtcaaagtgtatgatttttaaaaagacatatgcaaaatgaaatgtgctcaaatatgggttttgactgtcattttggatattaaggagaggagccaatcatcaggtgactagatcagagactagatacatggtactctaaagccttggactctacagatctcaaatcatggagttactttctacgaggcataaagggagccctttaatggtggagtatcaggatgtcagaaaggcttcccgtcctcagaaacatcacaaatgcttattaatttcctgaacttatttaatagaggaaacttcctttattttacccttagaaatatagattaaaaagcaattagcccttataagttcatataagtatgaacttatatttatacctacatataagtgaatgatagcaaacgttttctaaagtagattttaaagcaggaagaaaggagaggtgaaattctgaattcagcccttggctaatattgattatctagtaggtgtcagtactgtgctaggaactggggatacaaaagtgcttataatatgatatggaaattcacattttaatatggaaacatagaaatgtgtgcaaataagtgtaaatggaaggtaactgaagtcctaatagcggcgtgtgtaaagaacagtagaaacataaaggaggaagggtttgccaagactaaagggagcatttcagagaaacctgactgacctgatgactcccaattcagttctgagtcatgaataagatgttgacagttcttgaatgacatttcagatgaagtgcatgtggaatcatgagttattctgacatgttttcagaagtgctaggctgagacctactgtgctaggtgaatgatggtttggattttggcatgaacccataggggaggacctcctatgtcttgtaatagaaagtctgatctcttcccagggatgacgagaagccactggagagattcgatcagtctaattacctgaaagaaactttgctggcttcacagtagtgctaaagtctagtcctggcaaaaattatttaagactgtatccaactgagttccattaacaatggaaatggaaaggcagtgacagatattgcaccttaaatatagccagaacaaagtacttctaacaaagtatagacagctggaatctcaagctgaaattaagtgagctagagaaaaatgaagacatgatgtaaaaaggaggtagtgtgacaataaccttgtggaaaaggatcttgagttttcagagggtattataatacaatcgcagggtgtttatcttattttgtttcttttaggttatatatttgtgcaatgctgaaattaggatgtgaccatttaaataattcttagtagcgttaaattattggagatcaatatgactttgactgtcattcctagcaatgaactttttaatagaaaatgaaatttatctatttatcagcgtggtgttgtgcaaatttctttttgcttgaatatgttttaagaactagtggcaaatatttaaatctaatctttcaactgaaacatgactaaagagcttaaaatgtctattctcaggcacatttatggtgtgtcagctatccacctccatgagcatcgtatgctcaggagctcttatcaatacagagaacatggaaagaaaactccctgaatagaaatctcaatatctgtcacatattcttcattcagtcatttcatgttttataaaatgccaaaatatgaaatattaagtcatattttataacatgattgaatcacttgttatccagagtttaaaatctcatagacctctaatcaatgagtcagtgatgacaatgttacttatattttagacagagattctcaggttttttttttcttttttctgcctcaacagcaagtctttcagaacagtggctgtcaggtttccaatggtggtaattctcctggagaggaagtgggtttgggtagactgaggcagctccagaggtcatgcagtttgaaaaagcctccttcaataaagtgaataaagatccctaaagacatgcaggtctcaatccctggaaccttgtaatgttacctgatatagcaaaagggactttacagatgtgattaaattaaaaatcttgacattattcaggatgatctagatgggtcataaatgcagtccaagccttataaaagggagacagagggagattagactatagatggaaagaaggcagtgtaaccatagatgcagaaattggagtgatgcagccacaagccaaggaatgctggcagccactagaagctggaagaggcaagaaacaaatccatccctagagcctacaggaagaaccagcttggccaacacctgatttttaaccctgtaagactcacttcagactccagacttccagaagtataaaaaaaaagtttctgtggttttaagacactaagtgtatgataatttgttatagcagcaagaagaaacctatattatctcctgaattttctgatattccccagagttggggaatttttatgttttcagacaaactcattacaaaaaagaaaagaaaacaaaaaccctgaactttttatttttttattttattttttttcataatttttttatttcaaaaatgttatataaatggaatcatacagcatgtaatattttgagactggctttttttcattcagcataattcccgagatccacccaagcttctgtgtgtagtcgtagtccactctgtttttactgctgcatagtaattaatggtgtagatgtaccacagtctgttgaattattcacctgttgaaaaacatttaggttgtttataggttttccctattatgaataaagctactatgaacatttatgtacaggtttttacataaacttaagttttcatttttctaggtaaatgccatctgtgactcctgagttataaagtagttgtatatctagttttatgggaaactgccaaactgttacccagagtggtggtaccatctcacatgcccactaacaatgaatgagtgattcagttgctccttcagattttcagcatttggtgttttctctatttttattattgccattctaataggtatgtaatgatatctcatcgtggttttaatttgagtttctataacagctagtgaggaacatgttttcgtgtgcttatctgccattatttgcttatatctttggtgaaatggctctaaatgtattttgtccattttctttttattttattttattttattattattatactttaagttttagggtacatgtgcacaatgtgcaggttagttacatatgtatacatgtgccatgctggtgtgctgcacccattaacttgtcatttagcattaggtatatctcctaaagctatccttcccccctccccccaccccacaacagtccccagagtgtgatgttccccttcctgtgtccatgtgttctcattgttcaattcccacctatgagtgagaatatgcggtgtttggttttttgttcttgcgatagtttactgagaatgctgatttccaatttcatccatgtccctacaaaggacatgaactcatcattttttatggctgcatagtattccatggtgtatatgtgccacattttcttaatccaatctatcattcatggacatttgcgttggttccaagtctttgctattgtgtgtagtgccgcaataaacatacgtgtgcatatgtctttatagcagcctgatttatagtcctttgggtatatacccagtaatgggatggctgggtcaaatggtatttctagttctagatccctgaggaatggccacactgactttcacaatggttaaactagtttacagtcccaccaacagtgtaaaagtgttcctatttctccacatcctctccagcacctgttgtttcctgactttttaatgattgccattctaactggtgtgagatggtatctcattgtggttttgatttgcatttctctgatggccagtgatggtgagcattttttcatgtgttttttggctgcataaatgtcttcttttgagaagtgtctgttcatgtccttcacccactttttgatagggttgttggtttttttcttgtaaatttttttgagttcattgtagattctggatatgagccctttgtcagatgagtaggttgcaaaaattttctcccattttgtaggttgcctgttgactctgatggtagtttcttttgctgtgcagaagctctttagtttaattagatcccgtttgtcaattttggcttttgttgccattgcttttggtgttttagacatgaagtccttgcccatgcctatgtcctgaatggtaatgcctaggttttcttctagggtttttatggttttaggtctaacgtttaagtctttatttcatcttgaattaatttttgtataaggtgtaaggaagggatccagtttcagctttctacacatggctagccagttttcccagcaccatttattaaataaggaatcctttccccattgcttgtttttgtcaggttcgtcaaagatcggttagttgtagatatgcggcattatttctgagggctctgttctgttccgtttatctatacctctgttttggtaccagtaccatgctgttttggttactgtacccttgtagtatagtttgaagtcaggtagcgtgatgcctccagctttgttcttttggcttaggattgacttggtgatgcgggctcttttttggttccatacgaactttaaagtagttttttccaattctgtgaagaaagtcattggtagcttgatggggatggcattgaatctataaattaccttgggcagtatggccattttcaggaaacccatctcacgtgcagagacacacataggctcaaaataaaaggatggaggaagatctaccaagcaaatggaaaacaaaaaaaggcaggggttgcaatcctagtctctgataaaacagaatttaaaccaacaaagatcaaaagagacaaagaaggccattacataacggtaaagggatcaattcaacaggaagagctaactatcctaaatatatatgcacccaatacaggagcacccagattcataaagcaagtcctgagtgacctacaaagagacttagactcccacacaataataatgggagactttaacaccccactgtcaacattagacagatcaacgagacagaaagttaacaaggatacccaggaattgaactcagctctgcaccaagcggacctaatagacatctacagaactctccaccccaaatcaacagaatatacatttttttcaaccctgaactttttatagggtacaagcaattgcaaaataagaggcaatgaaatattctttaaaatatttgcggtgtttggttttttgttcttgtgatagtttactcagaatgtgggaattgaacaatgagatcacatggacacaggaaggggaatatcacactctggggactgttgtggggtggggggaggggggagggatagcatcgggagatatacctaatgctagatgacgagttagtgggtgcagcgcaccagcatggcacatgtatacatatgtaactaacctgcacaatgtgcacatgtaccctaaaacttaaagtataataaaaaaaaatttgcaatggtaccaaggctattgaaaccaatatgcatcctttaaaattagaataacccctctgttttgtaactttgaagttaatatgcataatgcttcatatactgagtagaatttacttacaatcttagagcaacaagggatttttggaagagacgggtttacaattttcagaagtgcctcacgcatatatgttattagaatcatttgacaacagagatccatggacagcttcctcaggggtgatatgatttgctagaggagccagcacagtttcattcatattcagatattgaactattgaacctcttaaaagctagaaattgaccttcactctatggaggaaagcaaagaatttgaacatttgactaattactgtgtctgtgaaactatgacgtttacctggttagctgtaccagttttatatcaggaaaaagaaacatgtcaaagtcagaaacatcccttctatgatgttttatattaaatttttagccaaaattcttataacattatcctgtcaggtgagcaaatgttacccttttggtgatgcttaagtaaaattacaattagataatgatttatgtattatttgtttaaaatctctctccccttatagactaataatttcatgaaggcaggtactgtgtgtattctacctatcattgtaaaccaggcatgttgcatggtgaatacttattattgaatgcatcttcttcacctatcccaattcttaaaattgagaaaattgatattgctttacttggaaatcaaattcaaaatagagtacctttttgctagttatagtaaaaaattctgtcaattatggagaagtattgtgcttgaggaaatttatgatacaatcactcttgatataattttcattgacttcatttaaaatttgtttctcataatatatatacaatgggaaatgtggcctttgtaatcatttttagtaggaactaagatacaactacataagaatttattaattataatgaaactctgaagtagaagcagttcaacttttgtctagttgctgctacatataccccacagggatgcctcacctaagaattgtgaatttatgtggaaaaaaatatatatctttcttttttcttccaactgaaatttatcgtttttgtcattgatttttttaccctgcccacaaaaatattagcagttgtgaaaactgtcaccagtagcaatcacatgtatcccatatcatatttatagttgctcagatttttttttgagtcaggttctctgtcactcaggctggagtgcagtgacatgatcatagtttgctgcagcctagaactcctcggcttaagggatcctcctgcctcagcttcagcctcccaagtagtggggaatataggcatgcaccataactactgactactattcagatatctttgaaatgtgttttacaggcattgctactttaaaattatgatagtttctaaacttattacaagagcttattacttaatgagttaataaagaaacatacatatttccatatctcatattttaaaaaataaatttgaaaattacatttcaatatgattggggtcctttaaattctatacattttaatttagaataattattatgggatagattcaccagactgctaaggagtggtgtagtagtccatttttacactcctatgaagaactacctgaaactgggtaatttataaagaaaggtttaattgacttacagttctgcatagctggggaggcctcaggaaacttacaatcatggcagaaggtgaagaggaagcaaagaccttcttcatgaagaaggaaagagaggcctagtaagaggaggggaaactgcctcattaaataatcagatcttgtgagagctcactatcatgagaacagcatggggaaaaccgcctccatgattcagtcacctctcacccaggtccctccctcaacacgtggggattatggggattacaattcgagatgagatttgggtggggacaaagagccaaaccatatcaaggggcatataataaaaaagattaaggaaaaactcctgccatagaagatctggtcagccaagatgcttttaataagagactatttttaaattttaaatctttcagttcatactaaagagtcttttcaagaatgacagagagatgtgatgtaatctaaatgttgcagatagtgtcctctgtgtgcgtgggctttccatacaaaagtataaatctaaaccaatgatatttggagacctttatctagcaagccttataaaaaaggagcctcatgtaaatttgaatatttaggcactaccattagtgaatgatagattgtgttgagttttcaaactgttccatgaagggaagagttctcattttttgtgaggttatgtcttaacatctgtttaagagtctgtatcctgttcattgagtatccttcctcatatccatcctctataactgcctttctttaacaaagccgtcatcagcatcctgtctttttccctaggttaaaaaggcaggaggcattaaaacaaaacttatcctgttttgaattgttagtgacatcagaaattatggatgttttcatcattgtttagattaggggatattttgagttaagagcaaatttgttttactaatcaggataggagaaatgacagtcctatagttgcatagtttggggtaaccagacagtccagatgaattacagatgaggtggcaaacaaaggctttctgtagtgttgtcagttagattgtgttatatagctgcaaataatacctgaatatgctgaaaatgacaaatgttcacttgctttagtgtccttggccaatgttctgcagcctcaagggaaaattgcaaattcatttttctttagtgcatttaagacgttacataaagatctcatagatcttaatttaaaataccatttacaaatggtcttttttatgtttttaaagcccaatttgcctataagttagttgatctcacgcgatgatgatatgtctagctggaactctatagaatcattttgttagccagcaacagctgtgctgttcttttctaaatagtaataatgctgtacattgcacttgtttttattgttgagaagggctcctggtacctttattgaggtactttgagagcagcctgatttcttttttggaatgcacaataaaatataagtaatttaaattttaatataccttccttacaccattaattaaaatgatttatgtttggttgccttcttgaaatagagtgtgatgttaggtctttcatgctttcatggtgatgctttccgacagagcatcttttacgagacagtatcaaactcatcctggtctcagtttatttgctttgacagaagcacctttacttaactcctggcagaatacactctcacactgttcttcttagttctcatttagcattctaaattatatttattgtccagatttcaggctacttatttacttgttcaaagtagaattatttgatcattgatgagtttgtcgaggaagaaataaaaagcagaggatgagataacttgaatcatattttggtcaggatgttgaaggctagatgttgaagttagatactttgtgggattcagaaaagaaggacttcacaagcagtaatgtaagaactttgtgtttttgtatgtcaaaagaaaggcattttacttcctctctgatcttatcaaaaagaaaagggagaatgaaaggaagacccccatatattttaaagaaattcttgtaaatactaagaatgtttctctattgtctgtgggtccctaggcacctagatgacattgccgataaattgggcattttttttgtcactggcaaaaatcagttctgaaatactacttagtctacagtcattgtacaatatcttttttttttaatccaaggcaattcaatgttgacagttcttttcactcttttctgaatattggaacatatgattgcatcaagctggctgcaggctccagtgctgacattttttagcgcaaagaatgcccatatcacttgaacatttgaaaagtgttaatgagcagatcaaaaggactgttgcctgaagcagctaacttttttttttttaaagaaatcggctctcttccaaactgaaatcttcattctagcagctaagtagatgagatgtaatccttcctgatgatgataataacaaatgaaaaaaaaattggagaagaatcaaactctgcatatggttcataaagtggtgaaagtgttttccagaatcttcaatacagagctgaaggatgctaagtggaaaaaattctggcaactaggcaatattatgccttttaaaatctgcaaattctcataaaatatggtgcatatgaatacattcttttctgaaatgaatgtgtattcttgcactatttgatataagaatggaattctcatgactgtagtttttaatgtatttttactaacagtaaatctttcttttctcttgaactagttttactgaaatgtaggaatgtcagttgaacacagatttttgccatttttggcttttgagccaaatggcttcagattattttcacccttctttgttactcatgaggttgtgtggcagagtggagagagggaagattttggaacatcacaagaaaaccattttgtttctcgtccccaaatttttatgtgggtgacattgggcatattagctaaatgttatatctatataaaatggtgataaactctatcttacatagttattaaaagttttaaatataactatctgtgtaattatctatcatctatttgtcaattcattcattcatccatccatccatccatccatccatccatccatccatatatccatccgtccatctacccatccatccatgcaagtaagtaacaactactcaacaaatcataggctttttcttctgcaccctttccctttgcaaactaaatcttattttaaaaagatgtcatatggcagaagttgaattcagagttggagataggtagacttggtcctcggtttgaatcctaattctgtgttttctctttgtatatatttgggcaagttattgaagttacagctttcctctccttatatttaaaatgaggataaacatagtgattgcacagagttattatgaaaataaaagagctatagagacagagcatgtggcatctggcagcaccaataaagggtggttcctgttaatagtgttaacaacacacagatgagccttagctctggttcagtttctgaaggtactattatttgttgtgatggtggttgtgtgccttaatttaccagatcaaaatgtgatttagatagagctttgagggcttcgttttcattattaacaatgtagaaaatatacttgtcaggactttaattcattttaaatgtataataagtcatgaattattcacacaaaagaataatggcatgctattgaaaagaactctaatgttcttttctggaataaggtgaatataattgcatatagacatatggccatatttatccatggaaaggcatagaagcaaaaacctggtggaaacattgtaatatgtattttttatcattttataagaattgacattttgacaaggacctacagttaatccaatttatttcccagatgaataaacttagtatacttctatctcctcttctacaattgggttatctgactttattggttcaggaaaaaatttagtgcttcattttcttagggactagtaatattttagtttcatgtaaatctatctgtgtattatttttgtatattttttcctcaatatttgacttatacatttggaaatcaactataattttttgtgaacgttctcagttccatcaatttcactttcagtatatttttagtacctttagtatctgctgtgtgcagagcactttattaggcatgatgacatgtataaataaggagaaataattattgccattaagaaatttgtaattttcagtgataatttaaaaattacaaaatcaacatataaacccaaattaatgatgcataaatggttcaactgtgcagcaacatatactttaaaaagttttatgtcttctagatgaaacaacagggagttggtgggctactctgtgaaggctggttgggacctaacaggaaaatgatcattgtatttgggggaagcatcaggatcatcaggctggctggagaggaggtgcatgtagggggatgtttgtagatacatgttaggccttaaccgtggagggactgacaccatgctgaggcattttaacaatgagaatagaaaaactggctcatgtttatgtgctgcaaaaggggctttgatagtttatcttgcagtttatattagataaaggacctccggtgaagagaaggaatgtaaagatgcctcccatggtactatgtgagtttgataacagtggtcatatggggattttaaattgaaattcaattttcccatatcactattaaactagatgctatatgggataatgaatatgacattaaaaaaaaggaagagttatggagagagatgttgaataattctcaggaggatgtaattctcaggaggaacagggttggggatactatttatgtcctcagaaatttatataacagtgcaagatgtacatgtaataagaagagtaaatttgaagtcctcacgatgaagtgaaaatgagttcagatgtattattctcagacttagtggaatggaaggatgtaccttgacctttttctagtgagagaactccagagagtactttatggaaattcacaaaactaagagtggaagcatgattcgtaaggtctttccaataaaatttagtactttattgagtcaaatttgaacattgggattgtttattcatttttaccagggaaatgatcgtttaagcttctgttagaagaatattgacctggctacataggttaaaatggggaatcaattggagatagagaaataagttagatgtctaatttaacaacaagtagagtattaagtcattgatgcaaaaaaggctatggtaattataactatcaataaattttgtgaatgtattattaattaaaattcttagaatgaggtggtgagaaaagatggagatgcatctcctttgtctgagcaccattcatttatttataatagctcagccatacttaaaatgttatcagttctagaaaccacactgaaatattgaccaaatcaatatgtgattaaaaaaaaaactgaccaagataaccagataactcaggaccatctcatgagaaatgtttgatatatttaggttccagattggtagactcagtagatatatggcaattatcctagcattcaaaaataggaactgctctcctctgaaagaaaaagtgcatgtgtgtgtgtgtgtgtgtgtgtgcgtgtgtgtgtgtgtggtgtattgccagactctaggatcaagactaaagactggaaactacagagtgggaagtgccttggcttcatttgggggaaaaactttcaaatgatgtgggttccttgccaatggactgaattgctttgtaaggtagtggatttctcttgattgcagtaattcaaacataattagactacagcttggtgggaacttttcagagtattgaagttttgattgtgtatgcgttggaggagttgaaaaaatgttgtactaggtacatttaaggcccaaagaatcttgagaatatgcttttaggagtgacagggggagggaatgaaaagaatcccttcttcctttgtccttccctctgcagtctatctccttccttcccttcctccttctttattctctcaatttgtttcttcctctcttctctcccttttttccttctttttttcctcctaatttccctttcttccttcttccctctctttctttatcccttcctccctccttttctctccccttctcacttctttccttttcctcttcctaacttctctttttccttacttccttgaaagtctaatttgtgctagacattctattaaatactagaaagtcagtgaggagcaaagcagacataacacctgttctcatgaaacttactatttttttgtagataaaattaataaacccttttttggtagataaaattatcaacttggataaattctaggagagaacaatgcatctgtgcatatcaagagaatcatatgctttgagaaaatggggagaattcatcagggagagaggaaggtgggggatatttcaaagaatgtattgggcatggaaatgatttggtaaagtcatttgaagtgagaaaagagattacagattatgccaactttttaggttgcaactgaaaaaagtcattcacttgaaagaaacagggaagataggaaggaatgttagcttttaaatgaagatggttagggtagctttgattttgttataaaatgctaacagatttggttttcattaattgttttggacatgcaagttcatagtcatatgacatgctttctttttttatagaggaaatatacctgcagtttgatgaacttaactttattttgttttattattattttaaatcttaattctctactggcatttatttctgggctaattataaaaaatcataaagttttatctttcatgattaccaaaatctactggaattttatgattattaaaaaaataaaattgaattaaataaagaattaagtagaaataatggctttgtagcattcaaaaggagattcaaatttaattactatttatatctatattattgtcattattgtctgtgtattttttccctgaggtttggatctaaactaaaatttagatgatgaaaacattatacacacagaaaattatgggtcagttctctaaacattatcaatgacatcaaaatataaaaaccctttcactatggaaaatggaaacatttacaaagagagagactaatatatgtacccacaaaaattaaaaattaaaaaaactaagataatgaacttcccactgtcttcattactcagcttcaatgattctcaacttattgtcaatttttttctttcaatttttattttaggttcaggggtacatgtacaggattgttacaggggtaaattatgtgttgctggtgtttggtgtacaaatgatttcatcacccaggtagtgagcatagtacctgatgggtagtttttcaaccctcacctttctaccgggttctcacttcatgtaggccctgatgtctattgttctcatctttctgtccatttgtactcaatgtgcagcttccactccacttataaaaaagaacctgtagtacttggttttctgtttctgtgctaattctctgaggataatggcctccagctgcatccatgttgctgccaatgacatgatttcattttttttaaatagctgtttagtattcactggtgtatctgtatcacttttttttctagccagtccatcattgatgggcatccaggttgattccatgtctttgctattgtgaatagtgttgtgatgaacatacaagtgcgtatgtctttggtagaacgatttatattcctttgggaatatacccaataatgggattgctggattaaatgtagttctgctttaagttctctgagaaatcttcaaactgctttccacaatggctgaactaatttatattctcactagcagtgtataaacattctcctttctctacagccttgctaactcctgttatttattttttactttttaataatagccattctgactggtgtgagatggtatcttgttgtggttttgatttgcatttctctaatgattagcaatattgagcatttttttataagcttgttggccttgtatatgtcttcttttgagaagtgcctgttcgtgtcctttgcccatttttttaaatgaggttgtttgttttttgccttttgatttaagttccttacagattctggatattagacctttactggatgcataggttgcaaatattttctcccattctgtagattatctgtttactctgttgacagtttctttcgctgtgcagaagctctttagtttaattaggtcctacttgtttattttttcttttgctgccattgcttttggaaacttcatcatgaaatctttgccaaagcctatgtccaaaatggtatttcccaggttttcttttagagttttaggttttatatttaagaatttaattcattttgagctggtttttatatatggtgaaaagaaggggtttagtttcagtcttccacatatagctagccagttataccagaatcagttactgaatagggagtcctttccccactgcttgttattgtcaactttgttgaagatcagatggtcgtaggtgtgtggctttatttctgggttatctaaccttttccagcagtaccatgctgctttggtgtactctagccttgtagcatagtttgaagacaggtagtgtgatgcctcctgcttttttctttttgcttaggatagctttggctattcaggttctcttctggttccatatgaattttataatagttttctctaattctatgaaaaatgatgttgatattttgatagaaatagcattgaatctgtaaattgctttgggcaatatggctattttaacaatattgattcttcctattcatgagcatagaatgctttttcatttgtttttgttatctctgattactttcagcagagtttggtaattctcattgcacagagcttttgcctccctggttagctgtattacaaggtgtttttttttttttttttttgtaactgttgttattgaattcttgatttggctctcagcttggacattattggtatacagatatgctacttatttttgcacatttggtttgtattctgaaaggttaatgaagttgtttatcaggtccaggaactttttgaaagaatatttagggttttctaaatatagaatcttattattggtgaagaaggataattttgacttcttattttcctatttggatgccttttatttctttctgttatctgatttctctggctaggacttccaggactttactgaatagaagtggtgagagtgggcatccttgtcttattctatttcttaagagaaatgcttccatcttttgcctgttcaatataatgttggctgtgggtttatcatagatggttcttactattttgagatattttttttgatgcctagtttgttgagagttttaacatgaagggatgtggaattttattgaaagttttttctgagtctattgagatgatcatgtggtttttgtttttagttctgttcatgtaatgaatcacatttattgatttacatatgttgagccaaccttgcatcccagggataaagtctactggatcatggtggattaactttttgatacactgctagatttggattgctagtattttgttgaggatttttgggtctatgttcatcaggcatattggcctgaagttttcttttttcattgtatctctgacagcttttgttatcagaatgatgctagccttatagaatgagttatggaggtgtcccttctcctcaactttttggaatagtttcagtaggattggtaccaggtcttctctatatgtctggtagaattcagccatgaatctgtctcatctacggcattttctggattttttttattactgatcaagtttcagaacttgttatttatctgttcaggataaatatttctttgttggtcaatcttcagaagtgtatgtttttaggaatttacccatgtttttctagattttttagtttgtatgcataaaggtattcctaatagtgttttagagttcttgtatttctttgggatctctaatgtcccctttgtcatttctgattatgcttatttggatcttcttttttttatttattaatctagttagtggtttatcaatcttgtttattttttgaaagaaccaaattttgattttgctgatcttttgtacagatttcatatctcagtttccttcagttcagctctgattttggttttcttttcttctgctagctttggggttggtttactcatgttttcctcgttcctctaggtgtgatgttagcttgttaatttgagatcttcctaacttcttgatgtaggcatttaacactataaacttttctctgaaaactgctttagccgtgtcccagagattctgtgatgttgaatctttgttttcattggtttcaaagaatttttaaatttctgccttaatttcactctttgcctgaaagtcatttaggagcaagtttgtttactttccatgtaattgtatggttttgagagctcttcttggtattgatttctatttctattgtactgtgttctgctagtgtgattggtatgattttgttgtttttaagtttgttgagaattgcttcatggccaagcatgtggtcgatcttagagtatgtgccatatgcagatgagaagaatgtatattctgttgttgttgggtattctgttgatgtctgttaggtccatttggttaagtgctgaggttaggtctcaaatatctttgttagttttctgcttcaatgatctgtccaatactgtcagtggagtgttgaagtctcccattattattgtgtggttatctaagcttcttcataggtctctaagaacttgttttatgaatctaggatctccaatttgggagtgtatgtatttaggagagttaagtcttcttgttgaatggaaacttttatcattatgcaatgctctttgtcttttttgatcattgctgatttaaagtctgtattgtctgaaataagaattgcaacccctcttatttttttgttttctgtttgcttgagtgatcgttctctattcctttactttcagcctacatatatatatatatatatatatatatatatatatatatatatatatataaaatttatgtgagatgaatttcttgaatatagtgtatagttgggttttgcttctgtatccaacttgccactctgtaccttttaagttggacacttagcccatttatcttcaaggtcaatattgatacgtgaagatttgatcttgtcatgttcttagctagttgttatatagacttgattgtatagttgctttatagtaccaatgtgatatgtacttaagtgtgtttttgtggtggcaggtcttacatttccatgtttagtgctccctttaggacctcttgtaaacataactgatagtaatggattcccttagcatttgcttgtttgaaaatgattttattcccccttcacttatgaagcttagtttggttggatatgaaattcttggttgaagtttcttttctttaaagttgcagaatataggccgccaatctcttttgacttgtaaagtttctgctgaaagtttcactgttggcctgatgggattccttttgtacttaacctggcccttctctgtagctgcctttaacatttctttcctttgcgttgactttggagaatctgatgactagatatcttatcttttctagtatattttaaagattctctgaatttcctgaatgtgcatgcctacctgtgtagtgaggttggggaaatttgtgtggacaatatcctcaaatatgttttccaagttgcttgttctctatctctttcagaaatgccagtgagtcataggtttgatcttcttacataatcccatatttcctggaggtttgttttttcatttttaaaaattcttaatttttttttgcctgcattgattcaaaggagcagtctttgagttttgagattcttttctcagcttcatctattttgtaattaatgcttccgattgcattataaaagtcttatagcaaatttttaatttccggaagttcaattttttttttttttttcttaaaatggcaatgtcattttccagctcttctatcgttttattagtttccttggattgggtttcaaccttctcctgtatctcactgggcttctccgccatccagattctgattctgtgtctgtcatttcagacatttcatttctgggggagttattgagatagtttgtggaggtaagaagacactctggcttttagagttgccagaattcttgtgctggttctttctcatctgtgttcactgattttcctttactttttgacattgctgtcctttggatgaagctatttgcttttatattctttgatgttcttgagggtttgaaggcagtataaattgtgtttagttgattgacttcatttctggatgctttcaggaggccaagcctcagcttagcactcctgagcttcatgctaatgccttggggactgggactgggcatatggctttgttctttgacctctcgaggtcaaacactttctgcactggaaggactggggtgttcccagactgctggcaaaacactctgatgggggctgttgacaaaagtgctctggttgtgggggcagggtgccacaggagaatgtcttgaagtagggggccaccagagagtgcacactagtggggcgatggtgtgaggtgcagacaaatgaaaaccaacagggcagcggggagccactggtgtgtatgcagtggtggagcattattgtcaatctttctttatctgtacccatatccaagtcccaacgcccaaattattttgatgaaaatatcaaaatcatatcatttcacctggaaatatttgtacatacctttaaaagacaaacagtctttaaatatatatccatgatcatagtaatatactacacattaaaaataattctttgatgaggtaaaagtggaaccacattttcaaagttgctattgttttaaacctttgaaaatttaatagcatagttgacacatttgacattagatgacatcaagctggaacattttcagttttccctcttagtatagagactttgatttgtacctgaagtagctgaagccattgacacttggtcaaagattttccatcacgatggtgtgaatatccagatagcccaataccagtaaaggtcatttcaaattaagccccattctattcacgtgaagaattaactgtattggggggccgaggcgggtggatcacccgagatcaggaatttgagaccagcctggccaatatggtgaaaccccgtctttaccaaaaatacaaaaaaattagccaggcgtggtggcgggcgcctgtaatcccagctactccagatgctgagataggagaatcgcttgaacccgggaggtggaggttgcagtgagctgaggtcatgccattgcactccagcctgggaaacaagagtgaaactccgtctcaaaaaaaaaaaaaaaaaaaagaattaactgtgaaagatttcatgagtaaggcatcacaggaaaggggtcttggctaagaagctgatttgttacaagcagcttcaagcaacagtttgtatccttacgggctgagttaggagactctcagttggagtctcattgtggttacagtcaggtgccagtttggccggagtcatctggaagcttggtgtgcctgagtgtccaagatggacttttctcttatatgtcccatatctcctctcgaatggctgaaatagctggggatcagctggtgtctctgtctcaccacccagcctaggttagtttgggtttcctcacagcttgatggtcttggggtggatttctgacctgacagcagtgttcctcaacagtgagtgttccacggatgtcagggaaaattaagttcttttgacctagcctgagaagtcacatagtaccacttctacctttttcttttttttctttctttttctttttcttttttttttgagacagagtctctctctgtcgcccaggctggagtccggtggcacgatctcggcacactgcaagctctgcctcccgggttcacgccattctcctgcctcagcctcgcaagtagctgggactacaggctcccgccaccacgcccggctaattttttttgtatttttagtagagacggggtttcaccgtgttagccaggatggtctcgatctgctgacctcgtgatccgcccccctcggcttcccaaagtgctgggattacaggcatgagcgaccgcacctggcccacttcaacctttttctatttgccgaaagctagttacaggcaatccaagattcaaagagaggggcataaatattgggtggcaagcttcagtgagggacttctttggagctaccacagtattttatgtatcaaattatttttacacatccttactatctcctctctatgccatagaaatttttatatttgattttttttgacaggttattaaggatcctccaccaccacccgcccctgcaccaaaagaggtaagtgagtgcctaaaaaactgatatggctgttttttttttccaaatgcagataccatgcccaaatttaatcaattttgtctgttagataactatgatacctatgtactgactttttaatttatacaaatcagtggttatttttcatcctcttatgatttatttcatgtgctagaggaatgggaacttcatacattactagatataaactggccattggaatcaatagtacataaagatttaatatacttttatagtactatatcaacagttctgatatcacttatgttattgacaaaaaacatccaagctgacagttgaaagatcctattactttcttcaaaattcttggtgcttttatatgattctgttaaccagaatcatgttaggaacatgaaataaggaatgtgctggtgttgtatattttcaaagcttctcaactgcctatcttttcagttcaaatttcatagagtccaaacctacacacacattgatgctttaatgactgttaaaatgctgtcctaatttaatattttatctttctaagtatgaagtgatgaatagatttgaaataccaaatgattcccatagttaaagaacaacttctagttacctagggatgatgtaagagagattcctgaaatttgaggagggaaaggagttctcatatcctgtagaactccacttgaaatttatgcatgtaagtgtaaatatatccacatgttctatacatagttttttccatagtaatgacataattcagaaaaatatttaaatatgagccagttccgactaaacagtattttaagaaattaattaaaaactatttataaaacatatttccacaggaggaagaagaacctttgcctactaaaaagtggccaactgtggatgcttcctattatggtggtcgaggggttggaggaattaaaagaatggaggttggtatcttaaaaaaaaatagaagctgctaatctggagtatgtatttctagggctattaagccatagaaactacttttcacaattcaatattataatatcatccatttgtgcttaacactgggtcttttctggatacatttttttttctatggaatcatcctagccgctgcaaaactggcctagaagaactggtttcattagtcaaaggattgcattgataagacctctgaattctaagggacaaaacaaagctagggtttttcttattttggaaaaaattaccgcatagaacgtcaactttgaagaagccagccacttcaagttgcaacaatgttcttctttgtaaagatccaatagttatctactatttgagaagaaaatgttaaaatattcaatagcgtggatctgactttatcacaaatatagaggaagcacatttaatactttatgttagttgccacttatgaactattgtaattgatattatatatcatttatagaatgcattgaactttcagactagcccaaagtgttgattaaaattgaactttaatattatatcttaaaagtcaaataagattttattatcaccatgaactattttttcaaagtgatatattatttagtaataagcaattattaccagcactgagctcttaacaacagtataaacaggcctgcatcccatcccaaggagcaattcaagggattttttcatgaacacattgactttattattaaattctggcattccttcctttttagtcataattttccttctgattagaatgtgttttatgcattttaccattgttgcacgtttcctattgtcatatctgctttgtgccagcaaaaattgataacctaatgaacaaccccacaattattgcaattttctccttttatacattacaataaaattcagattcccaaggagagtttccccttttcagaataaattttactcttcttcagaaaaagcccagacatactgaacatggttgtcattagcaaagtgtttatataggtcatctgacagtgattcttacttttctggaccacattagatcttgaagcaatggacattttccctgagagcttgccctttattgttgaaatatcctctgaggcagatacctcattgccaattatccatgattataaacagttgctagatgcagttaagctgttctttggagaagttattgcatgtatttttgtgtgctagaaactgagaatagcaaacaatataagagagcatggctaggcaagttcttgttgtttgacacagaaggaaggttattttcctttcttacctttgttgtgtcattctagacttggaaaggtgggaagaaagtatataggatgttcctatatgtataaaacatacacttcgaaataaaagtgttacaatcaaatatccaaatacgatctgtaacaacttatttttatttgatttcatctcattccaaaatgtgtgtgtaaaaagatctatataacggggaaaaaaggcttgatagaatataccaaaaatgtgagtaggtttctctggatggacaattttcaatctatgctatatatttccctattgtttaaatgtatttgacagtgagcatgtactacttcatattcagaataataaacttaatgccattttgaaattcactcatgtaaaatatggcatgataataaagtaaggatattagtatgtgagccttaattgaataaataatattcttgctttaaaaaactaggaatatgtcagatttttgttagttcaatattccagtgttccaaagattgagatctctttttaaaggaaaggaaatgtgtgagtgagcccctgaatgggtcctgagttaaaaggaacattattggtttatatttatgagagccaagagcatacacacatccctaactaacctataattcaagtttcaagagtttttaactctcaatatgtgaaattatgttgcccacacatccttttcagttgaacaaaatttgtgacgaaaaaccgcagccaacttgtaataaatctgggtccattttagttttggataattttggctaccttgcatggagtattccatcaacaagatggaaatgagaatgagaatgaagaaaaaaatggaaaagcaagtgtggatgttacaaaggggaaacacgatacacgttgagtatcccttatctgaaatgattggaactaaaagtgttttagattttggatttttttcagatttcaggacatttgcacatacttaatatgatatcttggggaggggagccaagtttaaacatcaaatttatttttgtttgatatataccctatacacataagaggaatatgtttgacacacaccttatacacacataagaagaatatgtatataaggtgtgcgtcaaacataaatgaatttaaaatataccataaatacacaaacgtaatatacaagcataaatgaattttaaaatatacaatgttttaaaataattttgtgcatgaaacaaagtgtcaactgtgacccatcagatgaggtcaggtttggagttttcctcttatgctgttaagttggcacttcatgggtttcaggttttagagcattttgaattttagatttttgaactatactcaacctgtatataaaaatacacagtaaaaattgaactgagaattaagcatcattaaacattttcaaacattaatttatttaaatgtggagatggaaatgtttcatttatctatttattttttaagaaatctggagtggatgatggggagttctcattatgttgccctggctggtcttgaacccctgagctcaagcgattctctcgattctctcatgtagctggggctacaggcaggtgccactgtgcaaacaaaatttgttttaaaagcatgcttattcaaacctggaaggtacttctctttttttttttttttttttttaaagcccttaaactaggaaaagtaaaaggaattatagaaattatgagcacatgtgaattacagaacacatttttatagctaaggttattttaagccagcatagggatttttacctagaatttttttcttccttcttctttaaattaaatatttaaacactatggtaatgggaatttttagaaagctggaacggagatgatgttaataacagctgttagcacaactgatccacaatacctaatgacttcaaatgtgtgtttctgccaattcagtgtgaaactgtttttgtttcttttctcgattctgaaattgctcctcaatgatataaccttctagtcatccaaatatgctctggaaattatggaattttattgcttataatgaaagccaagtggagttggaagaatttagctatcaagcttacacttatttttttccataataaagcctgatatattaattctgctaaggcttcctttagcagtctattgttgttattcttttgtcctttttaactgtctcaatgtggtcaaactaaaacattaaaatttatgcatacatacacatacacatatatatgtatatttaattgtttttaagagtttgaaaactgtaataatttctagtttcagactttgagtaagtgagaattgtttgtggtggtgtttgtggtggtgatgatgttgatgatgattaaatagctccctcatgaactaaaactaatgaatttctttaatggatcttctttagatctcagcaactatatttctcaagtatagtcatttgatagtgtagtacctagaaatgccacattgggtcagaactctagtttaactagcttcatatatatatttttttaatctgagaatagtgccagtgaaacttgaaagaagaatatgattatcattacctctgcaatatcaactgtgaaacataggcgagagagctgtctattctaaagcctatcatttctgaatggatctaaattgtatctacttctctttctaccctgaatgacttctctaagttaagacatttgtgtctttacttaattgtgtatcttttttttccttaaaattgagcctttacaactgcaaggatatgtgtcctgattttagagtttttaatacaatttagctatatctaaagattttataagtgttagtttatccaggtgatgacactgtgattggaaagagaaggtcatttctctggaaagaaagccaactatctagctctaaaggtttagtggtgtttaaaacaattttaacaacctcataaaaacattctagtatcattgatcacactgaaagatatagatacattttaatgactcaacagggcccacttgaaattgaaagcccattctaaaattgtagtaaaaaaaggtaaaaacatctaagtaagccttatcaaaaagactgaatagttctttccatgaatagtaatcttctctatccctacccactaattcagatcattaaaatgctttaggaagtcttaggatttttttttttctttttagaaatcacttaactctgatgtgtcatagtgtgccctcggtagctcttattataggtttttagagcttcactcctctgacctcgcccatttttggtgatgtccttggtttttgtagggctttccactactcctggttggacaagctcttctccaggggctgcatcctatttttacatgcccagtttgactcaaatttagctcttaatttctgtctattgaaaaatacaacaatcatttgctcttaatttctgtctattgaaaaataaaacaatcatttgatcttatggtttttcaatgtcaagaattttcagaggaggaaatttgctataatttgtggaattctctagggttcctgaacttttcttttcaaagcagtcttttcagatttgacctcctcaatgaaaaaaatcatatgccacacaaccatactcagatgagcttgggaactaaaggctgaacactatattacttagaactataaaagtaaagaaaactcatatataattattttactttcaaaaactaaatgttcagatcatactttttgccttaatcagatgtgatgaaaagtttggaagaagcaactcttttgtataaaatacatttgtaaaattctaaccaattggaaacttattaacccatttcaatccatgttcgaatccctgccctgtcacttactagctggctttgggcatatcacttaacctccctgtgcctcatctggaaagaagaaaatagagtctctatctcactgcatttttgtatggattaaatgcagactgacacatagtaatcactcagtaaatgctaactatcgtcattttcatatatttcttccagtgtcatcactacatatgggtatgtgtttagctagcaggggaacaaaccaaattagagaacaaaattacacactgtcatgcatttcatatgtaaaccacaaatggactctttatttaaaactatggaaaaatggttgcttcaaatttagttggctccagatttttttacaaagggttttttttttttttggatcttaatattaaatacagtcaggagtggtgagaaacctatttagcctctgaagaggagaaattaggaattaggatacctgctcatcttctttttcactctgagatgtgaactaaaggaccataggctcaataagagcctgttcctctaggacactttatgtctgataacagtactctccctttgttttacaggttcgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaaggtatgtgtccagattttgtgtaatttaactcaaaacttctggagacattgtaactttttttgtaccacatccccccagctacttgaaagagggtagatgaaattgcagatattcattgtacactcttaacagctgaatactttcttggcaataagaacctctaggtttttattacttattagtttaccttatgaaagaaatgagggtaaagaagattttactttcttcgttttatgtcttcatttattcagtacatagagattgagtacattctctgggaattcacaaatctcattttcccttagggtcgtcttgatgctctctgggctttgttgaggcggcagtatgaccgggtttctttgatgcgacctcaggaaggagatgaggtttgtatatgggaatgtattgagaaagagctaactgcttgagtcagtataatggaggcagggaaatagtaataaaaaatgattttaaagccctattgcacttgaggaaggaaatactgtcaagtgtagatttctattacatagaattgtctataaattacctattgttagaatgcatcttcaaactacatttttacaccattcccttttcaagaaattctttacaattctggtcatttgtacacatactttaataagcacaatcattgcatcagcccttgactttgaagacaaaatgttccaacagactgggagttagtagctgaagtccttctctagcatttagtagcatttagtagcaataaggcctagggcaaaggcctcagaatctgttttatagatataaatatgttccctgccttcaacagttgatgtaatcgaatttcttgactattacgtaaaagtgtctttaaagttataaagcattataaaaatctgtgatagtgataaacacacacacatacacacatatggatatgtatatattttttccccaccagggaagcctgtctgaggctatatttttgttttggaaatatggctacaattactctagttttgtaatttcagtaatatagtagtactatttcccattgtattgatgtaagaaaaattgattaaaaccttttcatatgtaacaatggataggtttggttcagagacagtacagttcataatagtagtgatgcagggcaggtgagccctaaaattgaaggttagcctgggagagttcttaacttcatctaagaaggaatctgatgcagggcaggaaggctcccaaattggagcttagcccaggaaggttcctggcttcatccagaaaagaattcaagggtaagccagaggtgttagcaacttttgttgaagcgacagtgcatagcagcagcagaggtactactccctgtggagcagggctaccctataggcatcgtgctcagaggagcagctcagaggcagttctgtagtcatattgatacccacttttaattatatgaaaattaaggggtggatcatgcagacatttctagaaaaggtgtgacaacttccgggtcatcgggtcattgtcatggaaagtggtggtaacttctgggtgttgccatggcaatggcaaactgacatggcaccctggtcagtgtgtcttatgggaaagtgtttttgccccatccctgttttagttaatcatcaattaggtctggtgtccaagcctcacctccagaatccagtactgcctcctacctcattagaattgttttcactgtgtgtcaacatgatcattcctctgttttaacaagggaataaacttaatgaacttcaacttcaggggattttttttttaaatgcgatttgagggccgggcgcggtggctcacgcctgtaatcccagcactttgggaagccgaggcgggcggatcacgaggtcaggagatcgagaccatcctggctaacatggagaaaccccgtctctactaaaaataaaaaaaattagcagggcgtggtggcgggcgcctgtagtcccagctactcgcgaggctgaggcaggagaatggcgtgaacctgggaggcggagcttgcagtgagccgagatcttgccactgcactccagcctgggcgacagagagagactacgtctcaaaaaaaaaaaaaaaagtgtaatttgagaaaatttattcctggaattttccccaacagatcatcatttactgtttgctaagaccataagtagagagaactagttggtttataaagcagtctctgtcatgatccaatcactgtggttcttcataatgacatttgtatctttttaaattcatctgtgatctggagttctgttctggaacaacatgtactaactcttcttcctcttctacatgctagactttaacttcatgtattttaaaattgttttcatgcatcatcttagtttttattccccaggttagacactagtttatttgttcaataattttcaccttattaagtgctgggtattgagctaacattgaatatatactagccaataaaaacagacagtggtctcagttctcttgctaatgctttatattttcttttaactgtcatgattgtaatttagtttatagtctttcattattcctattaattttgattcaattcaataatttttggattatgaatatgtaaaagcaatggcatcaagtaatatcaaagggtagaaacaaacagcagcctccgcccttaagaaacctgaactttgggaaacaagacatgtacaaaaataagatctattctggaatgtgttctgggtactcttgagtgcataggaaagtaaagattagctctccctcaggagagattagggaaagcttcttcaaaaatataggtgccctgtctttgaatataggtaggagatatgtagaatgaacatttccatagatgtggcatcatgagcaaagtcactgaggcagtgattcgtggtttgctagataatctggggttcagaagtgaaaaccagaaagattcactgggaatagaaaagaagaaatatgtgagtttagataattggtatgtttagtttttaaaatgatgcattataatcattcattcaacaaatatttacctagtgcctaacatatatcaggtaatctgttagatatcaaataaatttaacattcttcttaaattataatgctataatatttcacatgttgccccagcaggttaaattatttattttttcatgtttgaagaataatgcttccctttccattattttgaccaagattttcttcctttttctttttttttttaatcaatatgctcacacttttgtctaagataagtccaaagcctttattctaactcttgaggtcagattatttcaaaattcaggatttttctgattttaggaatatgatataatctatttatcaaatattatgtaaagcatcatctcctaatcatattattcagacatttttctattatatattaatatatatgaattgtaaaacttgttgcttccaaatgaattttggtgacaaatgtagggtaaaactttcagttttcaaagattttgaacctcagagatatagataaaatataattgacttttattatgtgaggttaactaaaaccccactttttccactctgtgctatttatgccttttttaaaatttgtttttacttaagtgcgggacttcaggcttagtaaacatcagttgtcgattcaattaattgatttgtttttttcatcagatacatattatgattctatgtgtgcctgatgcagtctgagttaatcttttatcgtcatacctattctttttagctttagtacttctgatgttttcttacctgcaaatttttatttcaagccactgttgaaacctcagtgtggcatcccatgagagccttttctcacagatggacacttcacagttaagtgtcattagtatgttaaagctaggaacctacccaaaagtacaaactcagtttactacatgattcccaaggatttttaataaattgtgttaaattccttgctgaatttaaggtataatgtatatctatctattcacccatccaaccaaaccaaccacagattcttcactcaagaaaatcaggtaagcttggcatgatttgccgttttgcctttatgtttcttgctagtgcttttttataatatttattattttgttttagactgaggttttatgtgaaatctgtagttatcagcatatgacctttaccttttagacaacttgagaaatcttcacccccatcccccacatttttatttcccagctttccattttcattgtttcacaaagattattaagaatacctgaatgtggccaggtgtggtggctcacacctgtaatcccagcactttgggaggccaagacaggtgggtcacttgaggccaggagttggagactagcctggccaacatggtgaaaccctgtctctaccaaaaaatagaaaaattagccaggcgtggtggggcacacctgtagtcccagctactcaggaggctgagttgaaagaaccacttgaacccaggaggtgaaggttgcagtagtgagctgagatcgcagtagtgagctgagatcgcaccactgccctccagcctggacaacagagggaaaaaaaaaaaatccaaatttatgtcaactccacatcatcaaccaggtcttctttatttcataaaattaactcccatataaattccctatcccacacacatataaacctgaacctacatttttccagagatttaatcataaaaatacacagcaagaattcttccactactttagtgaaatagctttctgtacattattaggataattgaagggccccaacattttttataagtttacagtatgtattaggaaaccatactattttggtttcaccatctctagagtgctatcttacttcccttcctccccaccttcctctttccctccctcttttccttccttccttcttcttttatctttgtttcttcttaaatgcatttatttccactatgctttcactttgttttgacaatttcttatactagtttatacatttttatatgttaattttcagttggcttcttgtgaacaaatgaagccttcggacgccctattctttttttttttttttttttattatactctaagttttagggtacatgtgcacattgtgcaggttagttacatatgtatacatgtgccatgctggtgcactgcacccactaatgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatactcatttgggtatatacccagtaatggaatggctgggtcaaatggtatttctagttctagatccctgaggaatcgccacactgacttccacaatggttgaactagtttacagtcccaccaacagtgtaaaagtgttcctatttctccgcatcctctccagcacctgttgtttcctgactttttaatgattgccattctaactggtgtgagatgatatctcatagtggttttgatttgcatttctctgatggccagtgatgatgagcatttcttcatgtgttttttgactgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttaattagatcccatttgtcaattttgtcttttgttgccattgcttttggtgttttggacatgaagtccttgcccacgcctatgtcctgaatggtaatgcctaggttttcttctagggtttttatggttttaggtttaacgtttaaatctttaatccatcttgaattaatttttgtataaagtgtaaggaagggatccagtttcagctttctacatatggctagccagttttcccagcaccatttattaaatagggaatcctttccccattgcttgtttttctcaggtttgtcaaagatcagatagttgtagatatgcggcattatttctgagggctctgttctgttccattgatctatatctctgttttggtaccagtaccatgctgttttggttactgtagccttgtagtatagtttgaagtcaggtagtgtgatgcctccagctttgttcttttggcttaggattgacttggcaatgcgggctcttttttggttccatatgaactttaaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtctcctcttttatttccttgagcagtggtttgtagttctccttgaagaggtccttcacatcccttgtaagttggattcctaggtattttattctctttgaagcaattgtgaatgggagttcacccatgatttggctctctgtttgtctgttgttggtgtataagaatgcttgtgatttttgtacattgattttgtatcctgagactttgctgaagttgcttatcagcttaaggagattttgggctgagacgatggggttttctagataaacaatcatgtcgtctgcaaacagggacaatttgacttcctcttttcctaattgaataccctttatttccttctcctgcctgattgccctggccagaacttccaacactatgttgaataggagcggtgagagagggcatccctgtcttgtgccggttttcaaagggaatgcttccagtttttgcccattcagtatgatattggctgtgggtttgtcatagatagctcttattattttgaaatacgtcccatcaatacctaatttattgagagtttttagcatgaagggttgttgaattttgtcaaaggctttttctgcatctattgagataatcatgtggtttttgtctttggctctgtttatatgctggattacatttattgatttgcgtatattgaaccagccttgcatcccagggatgaagcccacttgatcatggtggataagctttttgatgtgctgctggattcggtttgccagtattttattgaggatttttgcatcaatgttcatcaaggatattggtctaaaattctcttttttggttgtgtctctgcccggctttggtatcagaatgatgctggcctcataaaatgagttagggaggattccctctttttctattgattggaatagtttcagaaggaatggtaccagttcctccatgtacctctggtagaattcggctgtgaatccatctggtcctggactctttttggttggtaaactattgattattgccacaatttcagagcctgttattggtctattcagagattcaacttcttcctggtttagtcttgggagagtgtatgtgtcgaggaatgtatccatttcttctagattttctagtttatttgcgtagaggtgtttgtagtattctctgatggtagtttgtatttctgtgggatcggtggtgatatcccctttatcattttttattgtgtctatttgattcttctctctttttttattagtcttgctagcggtctatcaattttgttgatcctttcaaaaaaccagctcctggattcattgattttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtaggcatttagtgctataaatttccctctacacactgctttgaatgcgtcccagagattctggtatgtggtgtctttgttctcgttggtttcaaagaacatctttatttctgccttcatttcgttatgtacccagtagtcattcaggagcaggttgttcagtttccatgtagttgagcggctttgagtgagattcttaatcctgagttctagtttgattgcactgtggtctgagagatagtttgttataatttctgttcttttacatttgctgaggagagctttacttccaactatgtggtcaattttggaataggtgtggtgtggtgctgaaaaaaatgtatattctgttgatttggggtggagagttctgtagatgtctattaggtctgcttggtgcagagctgagttcaattcctgggtatccttgttgactttctgtctcgttgatctgtctaatgttgacagtggggtgttaaagtctcccattattaatgtgtgggagtctaagtctctttgtaggtcactgaggacttgctttatgaatctgggtgctcctgtattgggtgcataaatatttaggatagttagctcctcttgttgaattgatccctttaccattatgtaatggccttctttgtctcttttgatctttgttggtttaaagtctgttttatcagagactaggattgcaacccctgcctttttttgttttccattggcttggtagatcttcctccatccttttattttgagcctatgtgtgtctctgcacgtgagatgggtttcctgaatacagcacactgatgggtcttgactctttatccaacttgccagtctgtgtcttttaattgcagaatttagtccatttatatttaaagttaatattgttatgtgtcaatttgatcctgtcattatgatgttagctggtgattttgctcattagttgatgcagtttcttcctagtctcgatggtctttacattttggcatgattttgcagcggctggtaccggttgttcctttccatgtttagcgcttccttcaggagctcttttagggcaggcctggtggtgacaaaatctctcaacatttgcttgtctataaagtattttatttctccttcacttatgaagcttagtttggctggatatgaaattctgggttgaaaattcttttctttaagaatgttgaatattggcccccactctcttctggcttgtagggtttctgccgagagatctgctgttagtctgatgggctttcctttgagggtaacccgacctttctctctggctgcccttaacattttttccttcatttcaactttggtgaatcbchtgacaattatgtgtcttggagttgctcttctcgaggagtatctttgtggcgttctctgtatttcctgaatctgaacgttggcctgccttgctagattggggaagttctcctggataatatcctgcagagtgttttccaacttggttccattctccacatcactttcaggtacaccaatcagacgtagatttggtcttttcacatagtcccatatttcttggaggctttgctcatttctttttattcttttttctctaaacttcccttctcgcttcatttcattcatttcatcttccattgctgataccctttcttccagttgatcgcatcggctcctgaggcttctgcattcttcacgtagttctcgagccttggttttcagctccatcagctcctttaagcacttctctgtattggttattctagttatacattcttctaaatttttttcaaagttttcaacttctttgcctttggtttgaatgtcctcccgtagctcagagtaatttgatcgtctgaagccttcttctctcagctcgtcaaaatcattctccatccagctttgttctgttgctggtgaggaactgcgttcctttggaggaggagaggcgctctgcgttttagagtttccagtttttctgttctgttttttccccatctttgtggttttatctacttttggtctttgatgatggtgatgtacagatgggttttcggtgtagatgtcctttctggttgttagttttccttctaacagacaggaccctcagctgcaggtctgttggaataccctgccgtgtgaggtgtcagtgtgcccctgctggggggtgcctcccagttaggctgctcgggggtcaggagtcagggacccacttgaggaggcagtctgcccgttctcagatctccagctgcgtgctgggagaaccactgctctcttcaaagctgtcagacagggacacttaagtctgcagaggttactgctgtctttttgtttgtctgtgccctgcccccagaggtggagcctacagaggcaggcaggcctccttgagctgtggtgggctccacccagttcgagcttcctggctgctttgtttacctaagcaagcctgggcaatggcgggcgcccctcccccagcctcgttgccgccttgcagtttgatctcagactgctgtgctagcaatcagcgagattccgtgggcataggaccctccgagccaggtgtgggatatagtctcgtggtgcgccgtttcttaagccggtctgaaaagcgcaatattcgggtgggagtgacccgattttccaggtgcatccgtaacccctttctttgactcggaaagggaactccctgaccccttgcgcttcccaggtgaggcaattcctcgccctgcttcggctcgcgcacggtgcgcacacacactggcctgcgcccactgtctggcactccctagtgagatgaacccggtacctcagatggaaatgcagaaatcaccgtcttctgcgtcgctcacgctgggagctgtagacctgagctgttcctattcggccatcttggctcctccccggacgccctattcttatagtcgccagcacagtaaatatcttgattgacaaaggcaacttgtgccaaaaagaatgtgcagcaatcacaggccatttaatcatggtaaatatttcagtgatttttttaagtctctaaaataatgtaaacataaaaatagtattatccaaaaagtaatatttcattatttgtttaaataaaatcatatttgcatggggcagtgatataatctgtattgagactctaatatcctacaaattttatttctattttatagaaagagcacataatatttcttaaatttcagttcttttttgctttagagtatctttcatttggggaacttacagtaatttacctattcagaaaataatgacgcagaggcaggaagagtatcaggaatttagagtatatttggcacagctaatttggatgcatcatattaaaatgggatgatctgctttggtaatgctttatctatgcacgagaagacatccatagctcaccactaccttctacttactgtggttagttggtaaaaaattaaagttactgaaccttatggtttttaccaatttagtttatgtagatgtggtttatctcatggaattacatgtactaaagaaaattaaaatttaggggactacgatgcccaagtggtagtatcctctacttctctggctgttctttaaataactatctacctacatttatatttgcttcaagaaaggagaaaatgaattgtctttagagtagtgtgtgtgtgtgtgtgtgtgtgtgtgtctaagtcctagcataatcattcattgaatgagaaaaattagtcctggagttcttttgttagctttaaataatcacatatacctacaattgggctctaagcaagggtgaaatcctattgtttgcaaatttacatccctaatgtcaatcaaaatctgattaaatgaaaaacgttgaactgagaccaattcatgaaagattttatatttttaagagatgtccattgccctttgaaaatgtatatcacttttatatcaacaaatctatgttctcaacaaattatctgatattaggaatattaggaaatttgtaaatagcattgtatcagcttagattagatacactgattccctctaaaagaattgtctatgcatagcaaaaagttcactggaacaaatgttattgtctgttaaggtcaattctttttaaaataaactttcttatcaacatgtaaattggtgaatatatcttgcaaataattttattttcctgtgtgttctgtgctcataatggtttttgtgtgtatatatgcataatatttacttttgattatggttcttatgaagtatttatacaggtatacaagttccatgtgtgtgcttataaagcaatttttaaactgtagaaatcaggaagagtttaaacaatttttttagttagaaatggatggaacttgatgtgtattgtcaagaagtaaatgagaatgcttccttttttaaaggagataatagtgactctaaaaaaaggcaaaacaaacttcatgccacttgagagacaataaaacacagctgttttcaggttgtggctaatggttctaaataacacgacctctgcctagtcaacagcatgttttctacctaggaaaacaatcttgtcatttatttataattgtttactgcagattctgccgcttgaaactcaagtttagttgtataaacaaatcagcgttcctagaactcaacccagagtcacgcgcatttactctgtgagtcccgtcacggtgaatttccagccaaatgcgccccttttatggctcttatggctcggaggccgcaccgggcacgtttgtggaagaattcactgctgaattgaagtccttgatcaagtaattgaagttcaggcagaaagtatcaaagcgggcgagcagtctcccgctcgggccctctctcgcgctctcttccgcgcgccagcttccgcagaggtggcggggtccgcgggccgcacgccgggcccgggctttgattgcggcgggaagtcctccgagacatgaatgtttcagcgcggcgcgcgaggtgagggccgcgcgtccaggtgtaagctagcaggttgtcgctgacatctgtttgcagcggcggcagcggcctccccatcccgcagctagcggccggattgtcagcggaggtagttgagagcggcaaaaccgttttggtctcactctcgctgcctggaggcctgaaacaccaaaggtaggagtcggggtggacacagctgcgctcccaaacttgccttaggcacaaataagagatctaatgtagtttgactccgttgcggtggggagacgaggacagaaagccacttcagagacaacttaacgctgcaacatctgccgccaagtttccagggagtgacaggggacagcaggtacagatcgaagtctccacataaacaacctccattaagccgtggactcttttgaaaggcctccatcaaacatgccggagtccgcagcaggagtttgtttggttttcaaagcagtgttttgtttttcgcagtaacaaggtaatttataaatgatgcattaccatgcgttctcctttttgatttgaaaagagagagttagagaagcagaaaggcaaagaaagaagaaagcgcagcactagttttggagtagttccacaaaccaattaaatctccaaggagcttcttggctgtaatcgaggttatgttaaccttcagaaggcagtctctcctcaccagcaagcagacataaaagtgtgtatacagcggttgaagaatcctagtaacactcgtcaagtacatccaccatttgtttttaaacttttaaggtttcttattacacggagatgaaattaaattaaatctttgccgatggatgtaattttttactaaaacaaaatttacaaaattgaattgactttaactaattcctattcataaagacttcattcctatagttacatataaaaaataaaatgttaagtcagaagtaaacaaacattttctttttgggcggttctcctcctctactacctacattcagccagaaacaaaaaattcaagaatgtgttgacttttcaatggaaatactgcaaaaactccttaattttgcacacgtaaatatgacataatagaattggttacaatattatcctgataagaaagagctcctgggaatggtaagataataaagattctatagtaacgtaaaatattatcattcgtaaaatttaggctccctttcttgagaaaaaggagagatgctgactcaaaagtatcctttggagagggaagtagccagggagcaatggccaaagggcagatggtagtatgtagcaaagtttttatgtcttgttgaaataagcaacataaatatttttcagtatattgagaggacttttgtcagttgtgcagatgtaatttcgtttttttgaaatgattgacggcgattgctttctcaagaatattcagtctgacagaacactatctcaatcctccaattttttaaatctacagcaaaacccacaaaaatatcagagaagacaggttgcacaagttcatttaaaagggacccgagaatcctgataacgtcaaaggcaaaggctttgggctctacctcttgtttacatctctaatttctaaatttatgctcgtgcctttcagtcctgcttgtgaattttcaggctcacactggccttgccctttttaatggtggcttcttaaatgattacaaccttctcaaggatggctttgtactccattatgttaacaaatcacccagtgcttggcactcttaaaccatgtcatcttcaaatataataagcatctggatggtagagattctgcatcacactgccattatgttcccatcacagttgcgaagtgtaaccactagtgggtctttcagtgcagattggaaccttcactcaattgatttgatgcttcaagtgtcttcagtacctagaattatgctgctaatgactgcattcttaaaaaaaatcattaaaatatatgtatattgatagcctgggctactcaatattattaccccaaaaatcattgttttgaaaaagggctacatttttaaatttagtttttagtttccatccaaatattttgtgccatatagctgaactttgtaacagattttaggggctttagaaaaaataaccctttattgtagcctaattgagtcagtcctatcatgtataaaatgcccatgtatttctattcatttccttttatatattaaagtgacagagtagaaaatataaaaatgtttaaaaactgtaaacccattgtttaccttttatttctataagatctgaaatttgttatagtttctttagggcgattctgatgcaacaaagctggaaaagaataccactagtcttttccgctctttatattatctttcattattattatctttggcctttacctttcagggcgtctttaaaataccatcatattccatttaaattaatggaagtaattaacttcagtttatacatatttgaaagataaatttttactagaataatccatctcatgaacatgccatttttactactatcccactgactgttttaggagagtgtgtatatatatatgaatgtgcatatagcatgtttataactatatatatgcatatattcttataggacatatttgctcactgtgtatatcatgaactgtttcagtggaaaataacaaattcatgaaatagaatgctttttgataacatttcttagtttcaataaaataatctcttaattaggtatataagttggcaaaagtgttgactcagtctggtttcctgaagtgaaatgcagaatatgcaaaatttaatcatttaaattaccaaaaaatatattttttttcaaagaatctataaacagaacttgggtacttagttgtctcccctccccatataggcattattagctatttagtggagtcatatacattttctgtattaatttccaagtttagataaatatttatcttgagtaaaatttcatcagcaaccttttgataaacaagaaacaaaaaatgattagtgtaaatacctcttctcaatgcactggttttagaaatgtaacttcttttcatcatcatcagtgagctttggcactctgaaagaatacttttggctgagtgtggggctcacacctgcaatcccaacactttgggaggcagagatgagagaatcccttgagtcctggagtttgagaccagcctgggcaacatagggagaccccgtctctacaaaaaatacaaacaaaatattagcctggtagagtggaacatgcctgtagtaccagctacccgggaggctgaggtgggaggattgcttgagccagggatgtcaaggctgcagtaagccatgatcacactattgcactccagcctgggtgacagagcaagatcccatcttaaaaaaataacaataattaaaaaaagaatactttcaaaatgtcttggtggtccaggactaaacacattattttctgaggctcaacgtatggctgtgatgtagaatcaattaaatcatccaatggggagcagaaatgtcaacacagatacaattttaagctttattttccattttttggttactttatgtatggtttttatatactttttattacagaggcttaaatatatatttaaataaattactataatttctaataatttacagaaatctacattattctacatgtgttaaatactcatttttaaatttagagctcagaagacccagtccaaggggggatacagtgcatcttttttgtagacagtttgttacttaaatagaatgcattatgatggtgcctttgtgtctaccagaaccccatttataagtttagtttaaggaaagctttataactcagttaaggaaattggcttgagtaggaagcttttattgcccctttattcctaaaaatgtacttataaatgccttttcaactttgctaaatatctctcatttttatttataaataaaacactaggatttgtataacatagaagctaatatatagtagtatgcaatagtaataattatactaagtaatttctttaaaaatttttccatataatttttatttatattttccactaattgtactctgaatcaaattccaacctctaacatttaagaattttgaaactgatataattttgagttataaaataaaaataatttatttttcaaactttgcaaataacataaaaaaagagagaaggaaacagaaaataaggagaaatgaaagaatacccctggacatttgtttaccattttattttttctggctttgggttaacactttcttagtttcaaataattacaagttttttttctccttatgcttttaaaaaacttataaagttgggataatttctacaaactcattgccatttcacaataattttattctctgatatacatcatctctttctccttaaagagctcttttcacttggaaaatgtggcagaaagatactttaggttcccataatgcttcttagtaagcaggttagtatactgaaaataatacatcatatatattaaatttcataatcatatccagtactaagcatacttttgttttgaatcatattagagttttctaatttttttaaatcaagcacttttgttttctagagccatttcctgacatgaatgatttcttaatagttaattatgaattcactttattttatttaaatagctttaaggcggaacctagtgcagattggtgagacatacacacagacacatgcacatatgcccatctcaaatcttagttgcctaaaagagttagtaagtctgtaaaacatttcaataaaacgttcaaatatcatgtttacatacgtagcatacagataaatttctggagtaaagagggattactctcccattatctttctctaattctttcatttctctcccttgtctgacttcctaacattgattgaggcttccactgctttgtcctcattgttagagacgtcgttagacatcattcttagacaatgcgaccttccctcttgaattccacttgcatgtctctgagagcttatatgaattttccgctttgaccccctgttgtcactacattatctgtgtttagcgggacttagccattttcctcatgtatgtctttcaaattgtagttatataacttttctctcagtcatttgctctatccctcaccagatgtatctaaactgaggcccactagacttaagcacaccattgtggttcccacagctcagtgagcagggtcaagattttaactttctaattctgggtacagtctcctatgccatcagataacataattaccctgtgatatgcatgagatattgatttgaggaacagaattttgtctattatagttcctcttagttttattataattaagaatataaattataagttaatatgtgcattagatcctagtgattcataagagaggtatcatttgaatttgtagaatttaaacatttctgttatctttacttgttattttactacttataaattaagctactgagaaaaataagacagaacaattctgaaatttaacgtttgaattattttctgcaaaaatgtggctaaggatgatgatacaaaaaaatgttatttataaggggctaaaattagtcagtttggatgattaatttttaaattataattaaatctattgaacattttctgaattataaaaacataacacaatcattttcctttaggctaaattttgaacaaatgaaggcttttaatttttattgtctttttcagaatttttttttgcttgcagttgcatttttttcattttttaaaaacatttgatctttatttcatattctatttaagaaaaattttcaatgatcggaattcttgtgagatctgtatttgtcctagccaaatcatatattttaatgttaattaatcttttgcataatcatctatggtgctggcctatatgtggcttatgttagctctgagtgcctcattagtccagtttacagaaacactggctcttctcaacaaacagtgagaagagaatttaattcagttactactcttcagaagaggtgggacggttttaactgtcctccgaatccagatttatgaactagtcttatgctcttaactagattagttccttttccattaactgcttaaagtaggacagctgtccgagctgttggttgtaaatttgccagtgtctacttccttaatctcagatacaaaaagcagcagataacaggatcaaagtttcaggaccttcgtttttcacagcctgaaatgagcatggaggggctcatgaatgagagattgaaaagatgtgaggaagtgttacattggagaaaaagggatgacttctgtcattaatcttaattaattgctaagagttttaaatctgagttattaatactgatgttaagctcttaatacacttttcatgctctggcatcagatactcttttatgagataagaagtttaggaaggagagaatgtgtgtgtgtgataaaatattttcatttacatatttcaaatatttccttgggttaatattcagaacttcaccttttgacagttactgtagggcagttaggaatgatagacaagtaccaataaattcaaataaaatccctaggtatagcaacagcagtaaatctgaataatattactttcatgtttaccagcatacaaaatacttcactgaagaggaaacatcagattatgaggtgatatagaaggagagagaatcaagagctagaaaatgagacagaaaagaaaaggaaaaaagatgaagaatagggaagagatattattttaattaataatttgcaggcaataggaaaatgaagtgtcatttaaataataaactataaattatgtttttaaggttgtatatttcactattgcagccctaatcacttggaagtaaacggattgaaacttgaaatgactaaacagtcttttcttaaaattttgttttccttcccttcctccctgcgttgctgttccactaatgtttgtctagttaatacctttttaccattaagtctatattattattctgtgatatttttcaatgtgaaattgcaactaaccatattgtttatgagaattttggccacaaaaaatggcttcaaggggaaatctttaaaaaacagataaggctctaaacttcaaaagtatgattttcggattatatctcatgttctccatttatttggacaaaataaaaagaaaagctctgtcatgaaaatatagagcacactgaattattttagtatttacacatatatcttacctctaatgacagtgaagtaaatttcagtagtaaagtataatcagtagcttagatattcttaagttttctacaaatttaaaatcacaaatttggctttgagatacattatacttttctttttgcagcaatgttttcttcctttaatgattatctttaaatgtaagtaggaaaacatacaaatgaaacagtgttcatttgtttcatttatcaacccatttatttttgtacctattaattaattattgaagatgcaaaaatctgcaggcattaaggatttagattttatttatagtctgtggataataacagctttgaattggacaataacatgatgcaaaagaagttagaattagactcttggagggttctcaaatttgtaaaaggaatggaaataatatgtgttttcctgagtagcttagccctatctcaatgtttctgcctttgtagatagtctttcattatgggctccaaaaggaatcacataaaaattatgatatagcaggtcatattatatgtgatataactggtcaaattggtggatttggtggattgtcaagtggtcaagtggattcttaggtgaaagaacacttttttcctaatataaatccactctgttttttagaacacaaatttaggttcattaactgaaaaacagggattattggcttcagctatttttcttgatttgcccggggtctccttccttgcttcttgggaataaaacttgggtttccaatacttccaatgcttctatgagtgtgtgtgtgtgcgcgcgcatgcatatacttgtgtgcatggtgatgtttgtgtctaatgggaggtagcaaggtattgtagttgttgagggaactcataatttcaggagaggggagtatactttatttttatagagaagggtcagaactccgattcagctaacagtcactgagcacctactatgtcttatgtatgtttaattatcacatttttgtcctatctgtgttgagtcggtgtcatactgttctcatttaacagatgaagaatatgagatctagagatgattggtgaatttcccaagggtacattgccgaatgtaacagaatctagattgagagtcaggttttaaaatccagtgaacgctcgttattccatgctgtcattttacagtgcagctcttgattcctctacctattcaggcaataactgactgacttagggcaattatacatgtagggaagcacttttgaatttcagttttgaacctgattcttctggtgcctgggttatcaactgtgtatctgtgtttttactgatacacatatatttgacatatattcagaatgcatgcatattactctataatacacatacacaacatacatttaattatataggcatctataaataatatttaatatttagaagtggcaataaattctacagcaagtttatacagaataaggaatttttacatcacaagaaaccttagaagttgtgtaattcaaccccttcgtatactgcaacttccatccctacttcagttctctccaagagggacaagcgtgttcaaaagcacaagagggatgaactagcttatatattccaggaattgcacattgcttttcaaactgtagcaagagattggcagctgagaaggtgagttatgcaagagattaggagctgggaaagtgagttataggaaggaagaaaaattagagctattcctatagcccttggtggcatattggaaagaacactgcattgggagtcaggggagttgggatctcgatactgtcatttatcagcttcagtccggcgcaaattttctaaaccttctggtcttcggtcttctcatttgtgtatagaaaaataaatgatcaaaaagttacctttgaattctagcagtcttgtttttttaaaaaaatctaatttcaaccaacccagctctatgaatatgatatttttcctatgctcataagggtattcccaaaaccctgcacattgccctcagagaggaggccttaacaatatttgaacagaataattcatgcaggtggaaattctaacttattgtttagaatatgtttaaattaattcatactttttttagtatatagacatttcatgaggccagcaataatgaataaataaatattttacaagttactataagactaaaaaaatggcatatattctgcatagtatttgaaaatatgttcaagggactataaaaatactatgattctagatccataatggatatcttgctactggttacactttaaaataatttgaaaataactgcatatgaaagagagtaagggaaatacagattttaagagctctgtatatttatttaaattttattccagtttcctctctataaaatttgtctatttctcactgattttcttctttcaaaataagaagagatcaatgtgtgatattagattatctccattgtcattttgatatgctgtattttgctgtcttgtgtcacaactgtaaaataatttaaacacaagctatcatttaatactgttataagcaaatgagtactaatgtgttcttgtcattaaacacatatagatactggaactgttctcccattgggtcacattgaaattgattttccaacttgacctaacaataatggacaaatgttaaatgaaaatcaatcacttttacttaaatcagcctaatagactcattttattaagtggtctttttacctgcagctaactagatgacaaataacacttgtctagcctctgacctctggcctctgccctccggtctggcttctgaatgcgtttaaaaaacctctcagtacagatcatgattttattcttatatgacatattctttattaattagatgaaagtatatttcagatattttttataaatagaacaaaatcctgaattaatattatttcaatcacaaattcattctttttaaaatctggggtcttgctctcatctgggctggagtccagtggcacattgcacaaggctcattgcagccttgaactcctgggctcaagtgatcctcctactttagcctcctgagtagctagaactacagatgcctgtcactgggcccagtttttaattacaaatttttaggcccttaatatttcaaatatttcaaacatgtatcatgttactctaattatcaaaacatctcacagtaaaatatttttattgccattttaaagataaggaaactgagactttaaaatgtcaagtaagtctggacatggtggctcatacctgtaatcccagcattttagaaggccaaggtgggaggactacttgagcccaagagattgaggctgcagtgaccctgcacttcagcctgggcaacagagcaagaccttgtctcaaaaacaaacaacaactatatagagatctcaagttgccagattcatgcaggtaggtaggtaataagtggtattaactctaactttaaataggttattgaaaaatttagttcgtgtaaattctgctttatgtttcagaatttattggtcagttttccagaaaatttacagcagtttagagtttagtgttttagttatattaatatgttataggcccaggtgttgaattaagaaaaatttaacaataaaattgaaaaggccaacatataaatgagaagtactagtgaactatttttgtaacataggaatcactagtgaatatttaaatataatacatacaaaacataaatattaagtatcttagtcatattattgtaaatattggcgtcagaaaattatttgtggattgatttttaagcacctacagatattgtccagaggccaaaacttaagaataattataaagttatatactttacttaaaaaaaagaaatataattagtggtagaaaataaatcaacatgttagtcttaatattaaaatattaaaattgattgtcatttctaacctgtaaaatcaaagcaaaaggagttctatctggaggctaagattatgacacaatcccaaattatatatttcaattatcaaagctctcagatttactatggaaaagccatcatttttagtttaggatactgttggcacagtcataatttattatttataattccctcttaaaatctagttttcagaatctttcatcattttagacttccgttaaatttactgtgaatttactgtttatttcccagatcattctgagagcttgagctcttattttgatctatggagcaactgatttcactaatcaaatttattctttgatgtatctgaaaatagtgtgcccaaaatcatcttacaaaaattcaatcctgaaatggtttttaaagtcttaatatctcttcaggtacgttagtgaaaactgaggcaaatgggctgccagctatttttttgatgagaaatccttagtgtgtgagtactgggaactgagggttgagcatctggaggtcggcctcctcttcagtctgcctgccttcaacactaggtcctgtggtagacacctcagtccttacattccctatttcacatggaggggccaagggttccccatgatacagctgtcatagaacttgttgtttagatgaggcatcttgaaagactttcatgttgtggcatgctaatattatataatattttgctgagaaagaaagttttatcaaattataagaaataggaggtttcctttattcctagggcatcattctttgtactctctgttgtataaaataataatattactattattaataataataagatggtttttataactgaaggcataagaaaaatacccatgtaaactacattttaaggctttttatactctttagagcaaacagtttttctaatcatttataaacatttactgctttgtttctgaaatcttagaaataataaacaagtgaaattttggattaattaatagtattaatgaaaatacaacatattctctgtaagattacagatgaatattaaaacttaaattataagtgctataatttttctttttggcatgttggagcatttaacacatgagcatttaaacacatttctgacttttctaaatttttggtgaaaatattttctagtaagattagacaaaggaaaatgagctacaagttatcaacaggttctcgagaaaaaatttttttctttaaaaaatctagtgaatactagataatccctacctgctaggaatttattacagtctagatgtagtgggggataaagaaaagcctcaaaaattataacccaaagcagagcaagcaaaatgcaattaacaaataaaaacagaagataaaaccagttgtatagtcaagatggattctaattagaatctaatctaattagaattatatatatgatatatatttatatataaacagcaaatagtttatatatacctattatatatatttgaaaactccacaattataattccacatcaaataattttggtggtttaaaaaaatttgctagaattaggacatgataggcataaaataaaattagccattttttcctgagtacccactattttcaggcagtctttgaaaccccaaaatatttaatacatagttatcaacaagtttgtaatctactcaaaagggcaacaatccacatatacaacgtaaaataatgaaatatttaaacatcaattcaaggaagtaacagaagagattacacaaaagtgttgcctaattaatttctaaatgaattgtctggacattaattgctgtttgagttattaaggaagaatacttcagaccaagaggtaaggtgaggttttatagaagagagagttgagttaatcttgaaggatttgtagaggtagagaaagtaggaacatttacatttgttctgggacagtgattcaattattttctttagcaacttttgagctatatgtggggaaataatagagaataaatctaagtcccgtttggttgttctttttgttgtttttttttttttgttgttgttgttttttgagatagagtctctctctgtcccccaggctggaatgcagtggcgctatcttggctcactgcaacctccgcctcccaggttcaagcaattctcctgcctcagcctcctgagtagctgggattacagctgccaccatgcctggctaatttttgtatttttagtagagacagggtttcaccatgttggctaggctggtctcaaactcctgaccatgagtaatccccttccctcagcctcccaaagtgctgggattataggcatgagccaccacgcccagcagagtcctgttcagttctggagaatgtagtatattagactagcacttagctatgaatgtccaacaaatatttattgctgtttctcaactttaaaattttaaataaatctgtgtcccatcaaacaaaaatattttattgaattttgttttctaattttgtactaagacttcatgaaaacaagttgggttttttgtttgttttattttgttttcctacaagtattatcttggtgacaggcagtatactcttagaaggactgaaggtaagagcagaaggcaactgggctaatgcaggaaccaccttgttagggcccccatgagacagtagcactccatcagaatgggacaatctgagttgaaagggggatgctagcaggacttgattactatgtggagctaggtgtgtagcagagaagagagggagaggagagagaacctgaggctggttccatggagttcagcttgaaagaaaatactggaaatgctcagtagaaagttagaaatactggatttggcaggaacgtcaggcttggacatgcacactgaagaggcttttttgatcattcaaactgtagagtgtttaagggactgaggaacagtctggtctactttaataatgaatattttctctctcatgggtcaaaagtggcatctgttttgttttgttttttaaagtttgaaatagcaccttgacgtcttcttggcataatgttattttccaattctttgccatttttgatagcatggaatgtacaaaaactgaagtattggaataatccacaatgggagtggagaggggaaagaagaaatagtagctttggtttgaaatgttttgaagctattaaattgcagcagctattttcatagcttctttctttcaagagatacacttttgtagccactggagatgtggccttttttcttcctaaaatggtggaggtttttgtaaatcgacaagggtaataactaagagatgagggtctatggggtcttaagggccattgtatctgaaaaatgggtgttaaataatgacatttgtgaggaatagcacaaatagtaactattaaattcttaagtagactgcagaaagaatatgaatagagatgatatagtagttttgggctgtatttcttcaacttttttaggattgatgattcagtggttttaggatgactatttaatttaaaagaagtgaacagatattgaacaaaataataaatattgagtcagaaatttttagaaattgaagttttctaatagttttatagattttattattcaaagacaataacgttgatatgatagctgttcttgtcccttaatcataaaatttcaaatataatcatcagttatatactttatttttaaaatattggatgataattaataaactatagtatttgtaaactgatgtatttgcagcaaacatttgcaagaatgaaatgtatttactagcagaaagtaattttaggtaaaatttgatcaacttgttcagggcggaacttggcttaggagaaaactaaaatttattccataccttctacatgccatctattttgcattccttttcttatttaatcctcatatgaagtaggctttccactatgtatgttggaggttaaaaaaggttaaacagctggtaagttgtgacatttaaactcagagcttcctgacatcaaagcttcacttacattggagttagaaacttaaaaagatgatctaccaagaaataaattcttgggccactgaagacactgagatttgttcagtacatttctgtaagcaatacttatcatttgtacttttttaacctgccatattagcctgtacttttccaggaagaccatagcgataaggattatgacagtatggtcacccacctgggacagtgacagctgctgcagctgaatccagtccttgtcatcattaatgacctgcatttgacactttatcctagccatccttgtttagtgttcaatgtcttgcattggaataattactacctgtcaccctctatttaacagataactcttagtcatcccaaacatgtaagcatcatgtcataatgtgccaaacccatgttatttctgtgttctcatcctgtttgtgggtacttgcgtcctctgtctttccttttttataatgttcaaatacattttatacctaaattgatgaattttcattctgccactcatattaaaaggatttttcttcttagttttataataatagccagacatttattaagaggtaatattttctgaacataaagcatggtcactggatttagagtctctttatcctgttaaaaatattggagagctaaaggagaaatcagagaaaggaataatagtgctgactcaaagaaatgatgatgatgaaattcaaagtgtttaaatcatgtacagagcatatcataatgcctacattaaagactttgctttaattcaaagatctaggttacctacagagacttttctctgtttctctgtttctgtttctcctcctcctcctcctttccttcctcctccccttccttctccttctttgcatttagtttgaaagagagacaggagacagacaggcagggaattcgctaagttactaaagtggaaatggaagaaacctccatcacggttttaatcacgaatttaagtcatcataatggtgtatatttattgaatgggtttagaaattctttgtctcagagattgaaggttgagacttatttttagatttgtataatctttgttttgcttaaaaccctattcataggcatctgttatggtttattttacttagatgagttaatctaaaatgtgcttctgaatttctgtgtatcttaatttgacactttaagaaaacaaaataagtcatgcctgaaatttggagtaacacgtgttgtattttttatgtgacttcagctttattttcaaaagttcagattcaaatgtcagcttttatatttacgtgcttattggttttgattgagttatataacctgagtctctttttaacagcatttttcaaccaagtcctcttatctgaaccacaaaacagagaagataatttgagggggtattttcccaattctcctaaggatgccacataactagcataaggctttgatgagaagttagtttttaatatataacagatgcctttgagcactcagtcctaatgtactcatggaatcctggttaagaaacgctaatagtttaaaaaataattccatcttacatattttttccactatttctatcattaattaagaaaaagttttggttttgcaagctagaaatgagtacagtcaactagcaatttattgtattactcgaaaaggagatttctatgaagctaaaattaatttctcaaatacataccttctggataaagtataattttataacagtaatgaaaaggtagggccaaaatctgtttttacatgggcttattcaaatctatatttgtgaaattaaatttatgaatacattatgtttgggaagaagtcttttccagagttttataatatttactgacaaggtacttaaaagtggatactaattattgtgcatgtctatacatttttgcaataagttgtgttatcactcaaaagtgaccttgttagtgaggggaaaggcaagagagatgtatagtgcctcttgagagcaagtggagtgatgcgtatcaggagaagacactaaagatttattgtctgtggcaaaggaaacacaggtctctgcttgtgcagtaagtgtttttgacatcacccacagtaaagggtgattggaggctataggtcagaaaatgttctgctggggcaaaataaaaaactttttttttaacactatgagtttgtcttgctgtttcatctttgtagcacttaaaatatgaatgatggtcatgccaaaccaatccttgttgtgaataggaccttgctacccaaataatcagatttacctcactgaactaacatgcaagaacagattcagttataagagggggtagcaatacaaagcagaataattacttttttctttcaatttcatattttgcttatgaaaactttatttcctgtttacatgaactagcttaaaaactgataaatggttcatttagccttaaatttgaaggaaaactattctaatgggaagaactgaagaactgcgagtagcttatcttaatgcccttaaaacagcttcgtctgactgagaagtaaggactttgattcatttaatacagctgggttcctatttgcttctggccaaaagaagcaggtagcttaaacacagaagagattcacagcaatttggattattacctacatgggcatttaaatcttcagtaagaaaagataaagcatggtttcccttctgttttgtatgttatctagagcctaaggttttaggattagtacccttagctcataggatataaactgtatggtatatatttttctataactttaggtaacagcaaccattctaaacaaataaatgtctccaattaaaaacaaaaacagggagaatttgttggttcatcacctattcgtgtttttcttataaatgtattttattaaaagtgttttgattttctcgaattgatacaatataactgtagtgggtactatgatgtgctaccagatacccctgcaggatggagacactcattccctcatgccagaagagttggctactgacaatttgcagttgagcccctcttcaggaaattcccctccacccaagattacagcccctgcctggggctgcccatattcaatgattgattgatgtgagattgcaatggttggcctcctgccctaattaagaatagcttgaaaggccattccagctccagagcttacccgggattggctgagacctctgttaccaatgtgacacagtccaaattctccttttgccaaatcctactgcatttcacagaaatgggttgtttctgaaaacttacctcattaaacttctcacacacaaatctttgtcttagagtttgtttgcagaggcacacgacctaacttagtaacccatgttactatcttggcatacctagattccaattcctggttgtactttttactaaccttgtgccttgaagcaaattttcaatctttctaaacttccttttcttcttttaaatattttaaatggggatcgttatataacaacacttacctcatgtggttgtttgaagattaaatgagataataaagcataaagttcagtgactaaaacttggaaggtttttaataaatatttgttattattagttttaccatgcttacaattataattcaaaaatttgtatctctatttctctctgtcaccttcatcttacattttaccattttacatcttctactttgaaaaaacttactttctctgctttcagcttttttaaaaaaaaatttaggaccattattcagtgagcatatctggggcatatatttatttgtaggaacaaatatattaaagtagggcatattgtgatggctaaaatacatttgatttagattctagttggtttaatttagacctttctgactctgtggggctccttcatccattctcatcatatcagaggtgggatgactaaatcttctagagaaagatgtcaaatgaatactataaaagaattctgaatggctgtgatacaaaggtatctaacaaaagctatgtattttggtttcaacttgtattggagctctcaagtcagaaaatataaaatttctttcagctttattttatgttatataaactccctgctttattttttgtgagatttagatacgttaatacctgtgaaacacttaaaagattgcctggcatagagtaaatgcataagaactattttagccattgataacaatacataaacaatcagtgaatttcttctaataaaatgatgtaagatgaatgatggttgcaatgaaaatctgaaagctgaaattgaaatgggaattttaaaggataagattattccagggttaggttagctattgctaaataacaaaacttagggcatgttcctaaaaaagagatttccatactagatttttgtacatattgcttaacctacttagtgttcttctattgggcaagaaattgatgcatatttgctttctcaccattgccttcagtgaacaattttttatataagaaatgtggcagaaaggaaaaggcacatcatactcattagttaaataactctggtatagaagaatctatctccagtaaagctgtttgaaatcaaccccagatcatgagttaaataattcgggtaatatttttaaaaacatgaatttttctgcttcaaatgcatttatgcttgattttagaaaacttagagtagaaaattgaagtgttgttaggcaattacaaaacaactctgtaattttaattaaatttctttatcttcctgttcgcaggcgtcactttgtatgctgaaagctcaaactctagggagaaaacattggggtagaaaggagattcaaaagggcagaagagtctgccagcagtggtggacatgcttaatgtatttaaaagattaatgggctattgaaattaaaattgccagcacaaagcatgttgtaccaaactcacaaatatatgtgtaccaaccatatgggctacagcttagaccaatggcttacttttatattgtgacaagcatagtataggcaatttttttttattgtgaatgctgaaaaaaataccttttatttgtcacacaaattagaactatacacaagtataagaactcatgttcacatttttcccccaagcagtgcagtacatcatgtttttggtgtggtgccagaatatgatattgtaaaaatctgaacatttttctcctactacagcagtttaaaatggtcttgtaatttaatggtttttaataagtataacagtaataattattcctatatataatacaatattttagtcacaatggataacaaagtagcaatacttaatgtaatgtgttactttttcagcattatttaaatttttctgtaggggctgaattagttatagagagcttggcatgaaaatgcagaggcatttagagtacatttaatagacttgaattctagagctcttgcctcttctgcctttctgtcactgttctctgcttcacaaagatatcctctagagtaaaatattttgtaatttgtatgagcagcactaccaatgatattaggctttgcaaagcctctggaaggttttaattagcttgattcaaactcaggaatgaatgacactctgccttcagacttagcaagagaacttgccattttgttcagtgtcctttccttttcaacatcttcatctaaatggtcaataattatgtttataatcaaactcatgataacttttgcaaagtgttgaaatatccagttcctagaaatgaggaatggatagggcagtcatttatgattaaaaactagataaagcagtaaccagacatcaaaatatttcttgtttttgcttaaaaacaattatttagtattcttagggggttttattttttatttatttttttttttattatactctaagttttagggtacatgtgcacattgtgcaggttagttacatatgtatacatgtgccatgctggtgcgctgcacccactaatgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatactcatttgggtatatacccagtaatgggatggctgggtcaaatggtatttctagttctagatccctgaggaatcgccacactgacttccacaatggttgaactagtttacagtcccaccaacagtgtaaaagtgttcctatttctccgcatcctctccagcacctgttgtttcctgactttttaatgattgccattctaactggtgtgagatgatatctcatagtggttttgatttgcatttctctgatggccagtgatgatgagcatttcttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttaattagatcccatttgtcaattttgtcttttgttgccattgcttttggtgttttggacatgaagtccttgcccacgcctatgtcctgaatggtaatgcctaggttttcttctagggtttttatggttttaggtttaacgtttaaatctttaatccatcttgaattgatttttgtataaggtgtaaggaagggatccagtttcagctttctacatatggctagccagttttcccagcaccatttattaaatagggaatcctttccccattgcttgtttttctcaggtttgtcaaagatcagatagttgtagatatgcggcattatttctgagggctctgttctgttccattgatctatatctctgttttggtaccagtaccatgctgttttggttactgtagccttgtagtatagtttgaagtcaggtagtgtgatgcctccagctttgttcttttggcttaggattgacttggcaatgcgggctcttttttggttccatatgaactttaaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtctcctcttttatttccttgagcagtggtttgtagttctccttgaagaggtccttcacatcccttgtaagttggattcctaggtattttattctctttgaagcaattgtgaatgggagttcacccatgatttggctctctgtttgtctgttgttggtgtataagaatgcttgtgatttttgtacattgattttgtatcctgagactttgctgaagttgcttatcagcttaaggagattttgggctgagatgatggggttttctagatatacaatcatgtcgtctgcaaacagggacaatttgacttcctcttttcctaattgaataccctttatttccttctcctgcctgattgccctggccagaacttccaacactatgttgaataggagcggtgagagagggcatccctgtcttgtgccggttttcaaagggaatgcttccagtttttgcccattcagtatgatattggctgtgggtttgtcatagatagctcttattattttgaaatacgtcccatcaatacctaatttattgagagtttttagcatgaagggttgttgaattttgtcaaaggctttttctgcatctattgagataatcatgtggtttttgtctttgtctctgtttatatgctggattacatttattgatttgcgtatattgaaccagccttgcatcccagggatgaagcccacttgatcatggtggataagctttttgatgtgctgctggattcggtttgccagtattttattgaggatttttgcatcaatgttcatcaaggatattggtctaaaattctcttttttggttgtgtctctgcccggctttggtatcagaatgatgctggcctcataaaatgagttagggaggattccctctttttctattgattggaatagtttcagaaggaatggtaccagttcctccatgtacctctggtagaattcggctgtgaatccatctggtcctggactctttttggttggtaaactattgattattgccacaatttcagagcctgttattggtctattcagagattcaacttcttcctggtttagtcttgggagagtgtatgtgtcgaggaatgtatccatttcttctagattttctagtttatttgcgtagaggtgtttgtagtattctctgatggtagtttgtatttctgtgggatcggtggtgatatcccctttatcattttttattgtgtctatttgattcttctctctttttttctttattagtcttgctagcggtctatcaattttgttgatcctttcaaaaaaccagctcctggattcattgattttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtaggcatttagtgctataaatttccctctacacactgctttgaatgcgtcccagagattctggtatgtggtgtctttgttctcgttggtttcaaagaacatctttatttctgccttcatttcgttatgtacccagtagtcattcaggagcaggttgttcagtttccatgtagttgagcggctttgagtgagattcttaatcctgagttctagtttgattgcactgtggtctgagagatagtttgttataatttctgttcttttacatttgctgaggagagctttacttccaactatgtggtcaattttggaataggtgtggtgtggtgctgaaaaaaatgtatattctgttgatttggggtggagagttctgtagatgtctattaggtctgcttggtgcagagctgagttcaattcctgggtatccttgttgactttctgtctcgttgatctgtctaatgttgacagtggggtgttaaagtctcccattattaatgtgtgggagtctaagtctctttgtaggtcactcaggacttgctttatgaatctgggtgctcctgtattgggtgcataaatatttaggatagttagctcctcttgttgaattgatccctttaccattatgtaatggccttctttgtctcttttgatctttgttggtttaaagtctgttttatcagagactaggattgcaacccctgcctttttttgttttccattggcttggtagatcttcctccatccttttattttgagcctatgtgtgtctctgcacgtgagatgggtttcctgaatacagcacactgatgggtcttgactctttatccaacttgccagtctgtgtcttttaattgcagaatttagtccatttatatttaaagttaatattgttatgtgtgaatttgatcctgtcattatgatgttagctggtgattttgctcattagttgatgcagtttcttcctagtctcgatggtctttacattttggcatgattttgcagcggctggtaccggttgttcctttccatgtttagtgcttccttcaggagctcttttagggcaggcctggtggtgacaaaatctctcaacatttgcttgtctataaagtattttatttctccttcacttatgaagcttagtttggctggatatgaaattctgggttgaaaattcttttctttaagaatgttgtatattggcccccactctcttctggcttgtagggtttctgccaagagatccgctgttagtctgatgggctttcctttgagggtaacccgacctttctctctggctgcccttaacattttttccttcatttcaactttggtgaatctgacaattatgtgtcttggagttgctcttctcgaggagtatctttgtggcgttctctgtatttcctgaatctgaacgttggcctgccttgctagattggggaagttctcctggataatatcctgcagagtgttttccaacttggttccattctccacatcactttcaggtacaccaatcagacgtagatttggtcttttcacatagtcccatatttcttggaggctttgctcatttctttttattcttttttctctaaacttcccttctcgcttcatttcattcatttcatcttccatcgctgataccctttcttccagttgatcgcatcggctcctgaggcttctgcattcttcacgtagttctcgagccttggttttcagctccatcagctcctttaagcacttctctgtattggttattctagttatacattcttctaaatttttttcaaagttttcaacttctttgcctttggtttgaatgtcctcccgtagctcagagtaatttgatcgtctgaagccttcttctctcagctcgtcaaaatcattctccatccagctttgttctgttgctggtgaggaactgcgttcctttggaggaggagaggcgctctgcgttttagagtttccagtttttctgttctgttttttccccatctttgtggttttatctacttttggtctttgatgatggtgatgtacagatgggttttcggtgtagatgtcctttctggttgttagttttccttctaacagacaggaccctcagctgcaggtctgttggaataccctgccgtgtgaggtgtcagtgtgcccctgctggggggtgcctcccagttaggctgctcgggggtcaggagtcagggacccacttgaggaggcagtctgtctgcccgttctcagatctccagctgcgtgctgggagaaccactgctctcttcaaagctgtcagacagggacacttaagtctgcagaggttactgctgtctttttgtttgtctgtgccctgcccccagaggtggagcctacagaggcaggcaggcctccttgagctgtggtgggctccacccagttcgagcttcccggctgctttgtttacctaagcaagcctgggcaatggcgggcgcccctcccccagcctcgttgccgccttgcagtttgatctcagactgctgtgctagcaatcagcgagattccgtgggcgtaggaccctccgagccaggtgtgggatatagtctcgtggtgcgccgtttcttaagccggtctgaaaagcgcaatattcgggtgggagtgacccgattttccaggtgcgaccgtcacccctttctttgactcggaaagggaactccctgaccccttgcgcttcccaggtgaggcaatgcctcgccctgcttcggctcgtgcacggtgcgcacacacactggcctgcgcccactgtctggcactccctagtgagatgaacccggtacctcagatggaaatgcagaaatcaccgtcttctgcgtcgctcacgctgggagctgtagaccggagctgttcctattcggccatcttggctcctctcttagggggttttaaaaacactatcacagtgctgggtacataattgatgctcaatatatacataagcattcaagttgttaccttgttagtgacacataacattttatgatttatttatttctagtttagataacggaacaaatctgtttgagtatggtactatataccctgatagacagtacctctctcagggtagatacaaattgcaaattagagttgggaaaaagtggcccaagcaagtggcacaaaaagtcacttgcagataaaaagctttctgtgagtatgttacagtgaaactttcaatgaagatatgtccaaatgcagttaatcagaaaccagccaaactagcctaaacatttatggatattacaaacattcttccaaaaattttttttcttctgctcaattttgtgctgttttaaatgattatatgtaattaattttcaaggaacttagttgagccaaatgtgaatcttttgaggttttcctattgctagcaattgcttcattaagaatatgaaataggcacctggtttttttaatgctactgttgagaatacagtgaatgagtcctatttataactgaattaaaagcttaattaagtttatgagtgttttaaaactgtataaagttaatgctaatgtatttattaaaagtaaaaattattaatacatttttcttctttgcaacaaagattaaaatgaaaaatgctttaaaatgattgaatgaatacataatcaattatccattgcttgctgactttctgcctagcattatacaagtgatagaaaaaaatagaaaatataagtgtagtttttaccattggttagtacagtggtagagtgtagtacctttagtgtggtactactagtgtagtaccattgtactaggtgggaatgattataagcacaaaaaaggtgattataacccatagtgcaattaataatcagaacttaaagtgaattagtttaaatattgtgcagtaatttttaaattaattgttttctaattatttcatgtgttagtcttttcttcttcccttttacgtttaataaaggattttttttatttcttttgcattatttcccagtccctgagaccaacttgaatactgttgaaatatttcactaatttctcccatcaggaattatacctaagcttttatgaggacataaattaccttgaaagaaaaggaaacttttggagaattagaaaaatgtgagaatgttttgattgttcatttaggttttggttgtgagatgaaggagaaaacttgcataggaagtacctaagcagagttctaatgcagtgcaaccagttttagtgctaggttgtgaaggagaatagtaatggttttgaaagagagtagctaagctctgatgacaggaggtctgaaaataagacagttaaatagcattttgtcatttaaaaatttatctgaatagagatcaattaaatattctgcaaataagaatgcaactaattatatcatttgtagtacttgtgtaatagtgattaaaacatctgaacccttttaaaaagtggtattgtactgatagtaaaaataatgtgtagtaactctctgcattgagaactatgaataatatctcaaaattagaatatatgactcctggctagaggttgtacacttgctaaatcctaattaattccatttttttacaactttggaatggaatcatagtttaaggtgctcttttgttggaacaggcatttattgtttcagaagagagattccactgggagcggagtcatttccttagagtacattagcctaagtgtcactctttaacaaagctatgcaacagctggaccacaatagaaatttactatattttattgcaatgaatgtgaaatatcagtgttccttccaaacacaagtagccaacaacgtacacacaaatgggcagagctaaccattgctaattcatcttagatttccaagtcctttgattcaagtataaaagtgttttaaaatttcttcacattgcaatagctgtatccaaaataaataagactttcttctcactatcccacatgccttagccaggcaattatattaataaatgtcagtgtactatcttgcactttgcttcttcttcttaatgttttgactcaggcttcaccctagttaaagctccttctggcattttctccagttttatggctcccttttttttttccagttcagctggcctttgagctcctcttttgatgttcagtatgtttctctagacagtctggccccaactctttgtcactaatttattttcgtattgtgatccatgatatacccccacaggctttaaataatttcaagaatgctgtttatagttgagcaaattataacctgcataaaggtggaaaatgagggagtaatggagagggtgggggtggggggactgctgaaatccagcctggaggaaagggtgctttttctaattggtcaatggtagtgctttaaaaaaaaaaagttggggggcgatgacatgtaatttttaaactctccacctttgtagaggtcatatactttaattttaagggcgtgttacaaacctatcaaatttcttagcaaaggccagcattattcagttgttttttttttttatactttaagttttagggtacatgggcacaacgtgcaggttagttacatatgtatacatgtgccatgttggtgtgctgcacccatcaacttgtcatttaacattaggtatatctcctaatgctatccctcccagttttcttaacagaaggcccaaagtcagttcctgaaagcagacaaggtctctttccaatgactagtgtgtatccttcatgaaagaactgctgagtagacagttttttttttttttttgaaacttgactacttggaggagttttttgtcctaacatctattttaaagttttatttaaaacaagtctatgtattaaactattttccagttcagatctcttagacagaaatagtagcaatgcatggcgtttccttagtgtttatcatatatcaggcattaagcttcatgcacattcacataattctcctggtaaccctctggtaagctgctattactttccatattttataaatgatgaaaataaggtgaaagagtcaaaagagtcttaaatgataggtttggtgtttttttttttttgttgttgttgttgttttagacagggtcttgctcagtcacccaggctggagtatagtggcatgatcttggctcattgcaacctctgcctcccaggttcaaacgattcccatgcctcagcctcccaagtagctgggattacagtcatatccaaccatgcccggctaatttttgtatttttagtactcagggttttgccatgttggccaggctggtcttgaactcctggcctcaagtgatttgcccacattgcccccacaaagtgctgggattatataggcataagccactgcacacggccagatttggttttaaaagacaacctacattcttgacaattcctgtcatcctattcaaaagcattgatggtcatttactgatactcttgtcacagaaactctattttattgctttttatatacacatttttatatgctacagcttgtatgtgagaagaaaactcagaattatgaatggttattcatagtttctaacaatagaaacaattggccaggcgcgatggctcatgcctgcaatcccagcactttgggaggccgaggcgggcggatcacgaggtcaggagatcgagaccatcttccctaacacggcgaaaccccatctctactaaaaatacaaaaacaaaattagccgggcgtggtgttgggcgcctgtagtcccagctattccgaaggctgaggcgggagaatggcgtgaacccaggaggcggagcttgcagtgagccgagatcgtgccactgcaccccagcctgggtgacagagtgagactctgtctcaaaaaaaaaaaaaaaaacaaacacaccagggcctgttgtggggtggggggagggtggagggatagcattaggagatatacctaatgttaaatgaagagttaatgggtgcagcacaccaacatggcacatgtatacatatgtaacaaacctgcacgttgtgcacatgtaccctgaaacttaaagtataatttaaaaaataataaaataaaacaaaacaaaacaaaaaccaaaccaatatctttcttccaacaatatgattcagtggtgagattacagtggataaaaggtaacttcagattttttctgtggaatgagatggggtaaagcatacaaataactaaggtggtattgttttctttttaaaatacaaaacaaatacctatatattaaagacaaattgcaaaagtttctgaaggacagagaagaaataattggcacctgatcttttttgtgaacctgttttttgaacagaacctgttttctaaaacttagttgaatccatacaatataaatactgatttctatcctacttttaaattatgccattattggtttttatattatgtagttttcatgatagcagtcacctggcatcttaccaagcagatttactatagtccacttaacagtcctctgtgttaggatattcaatagtttttcaccgtgggaaggcaccattataactaatgcagcaaggattacttttattttaagtgggggtattgttaaggagaaaataaataggcttatgatggagaatttggaaggaatatctttttccaagaaaaatgtatcactcctttatgccactaaggagtgatgagccacttaaataattatgcaagggaatcctttctgcaatgtagttgctaccctcctgctgcaatgcctccttcactaatctagttctctgtagactgaatcacatgctaatttttttttttttttttttttttttttgagatggagttttgctcttgttgcccaggctggaatgcaatggcatgatctcagttcactgcaacctctacttcccaggttcaagcgattctcctgcctcagccttccgagtagctggggttacaggcgcccgccatcatgcccatctaatttttgtatttttagtagagacggggtttcaccatgttgacgaggctggtctggaactcctgacctcaggcaatccacccgcctcgcctcccaaagtgctgagattacaggcatgagccaccactcctggccatcacatgctaaatttttaactcttttcttgctgccactcttcaagaaacaaactttaccatgggatagatacaggcaagattatgaaccaaaattaatgttattaatattttgcttgtatttaaaatttaacaaaacatgcatgcttaaggccttggttcgtggtgggcattccaattagagtatggtaatatgacttggtcaaaaaaaaaagaaagaaaatataatcttcaggataacatatcaacagtatacaataaattcaaatgtatattagtgttatttaacatgtaagcatatattatgtatgagtgcctttattcatccgtttattttctttaatattcatttaattgacaatgtattaccaggcactgtgagagatcagtgaacaagatatatcaagtctctctttttgtcgggtttacctctagctcaagaggcagataaaaaaaatcagaatacctatgaaatttgaaataatttgaagagtgatatgaatgagaatatctgtaggcggcatattttggtacattaatgggcaaataatgatatcaggtagcaagggctatagagaaaaaaataaggaaataaagaatggttgggcagagtcagtagctattcagatatacatgaatacttgagtgtgttctgacagttgcacaaattaaattatccacatttagtattatttctaaggatatatcatttatttgaataccatctaaattaatgcatttgtggcaaataaattaaatatgctcatttgcttgagaatttctattgttgatctgggaacagacagtgagctctcacagttgcagctttaaccttgccaatcacagacttcctgtaagtgcactgcgttatctggaaccttttcggggttggcgctggcattcttttactagattacatttatgcattagtgccatctgtgcctgctgcaccttctgtgagttagccctggccaaatgactggcatttatctggaagttggagtgggggtggggtgtttggggagaaagagagagagagagagagagagagagagagagagaagaagcgaggaaatcctagaacattttactcactgttggcacaagtggaatttaggaaagaagacacattggtaagcacaaaatatttgcagctggtgtccctgacttaaacccttataaagctaactctattcatttgctctctcttgaaaaataaaatataatacctatttatttttaggcaatgatatgcaaaagaagctaaaaccaaacatttaaataaggaagtacaacaaagcacatttgcaagattttcaagtttggcaaaattatgtacacaatttgaatatgtgtgcacttatatgacaaatatacataatgtgaggtttggtttagttgattagatttggtagaccactcgaattggactttgtattgataaatgggctgaaatgtagatttactaatattacaattctatttaggaaaattatggctatattcaacatataatcaggatactaaattattctgtagaatctttctgggattatattattttaaattgtattgcaaaatagatcaacaactctcatatctataatacactttagagctgtaagcggaaataaatagataaataattctccaatcagtaaaaatagatgttgaaaagtttcagctcgtttacactatatctatttttatcttatacctacttttagtatgcttgattatctggtttccatactgcttccaaattagaaacagtgaaatagaattggggcagaggggctgctaaagatgggtactacacgttagtgggaggtaatagttgcaaggaaaagaaggagagaatgagaggacagacacatgactcagacaagcactgcagtatggggccattcagtgcaagaagaaacagcttcttattccccagtatgccctgacagcactgatggaccacatcctgataatcaacgttcataattataaaactttatcttcagcatgaaagcatgtgcagacaacacattttaggaagatggaaatgtgaatatgttcttgaaaagaattccatccaaaatgaaaaatatatacttttagaacttaatgtagttaatccacagctttccttgagtactagcttgtgttgtactgtaggttttagggatcagagttcactttctacggtctaaaataggtaaggcctctcttatataagttctcctaatatatcttatattcatcttcataatacatttcagatttaaattgtaatgtatttaatttctgtaataaaggtaggcttttttctgttttcatttgataatattttattatctctaatatagttttaaaagaactatacaagtatccaagacatagaaattcattagtgctatagtgattatgattatgggtgttggaggttgattcttctgtgtgcaaatcttggtcctgccacttacttgctaggaagatttgagtaaatgacttgcccactctgagcatcagtttcctcatctataaattcagtataatagtacttgtcacaggaccgttgtaatgattaaatggttccagagcttataaagcatttagagaagcacttggggtataaaaagaactacattgtatgatggttagtcatttttattattgctcttgttgtcggaaaactattatatacatgtggaacttagaaggttatagttcaaattatactttaaaagatgtttgtaactggtttccctgctttacttttttttgtatatttgcatatatatcagtttggaaaggtcttcagaatcacaacagtattgttctgttagctgtaggggaaaagatgccataacgtaaaatgcctgggggcctatctcagctgagaatcttgtccttgcatcaacattaagaacagctagccacaactttattctttctcctctttcactgacaacactcagaatatttacttttgttgttgttgttgttgttgatactgagtctcgctctgttgcccagcctggagtgcagtggcgcaattttggctcactgcaacttccacctcccaggttcaagcaattctctgcctcagcctccagagtagctgggattacaggcatctgccaccacgcccggctaatttttatatttttagtagagatggggttttgccatcttggccaggctggtcttgaactcctgacctagtgattcacccgccttgtcctcccaaagtgctgggattacaggggtgagccactgcgcctgccctcccaacacgcctttttaaaatttatttttatttttttatattttatggccaagtctgtgagcccagtagtggtcatctatttccaccagaaattgtaggcaatagacagggagacccgtattatggccaacttaaacgactgagagaagagggctggtgagaaaacagcaattcttttgccccgcactctaaattattaggataattgtaccctgaaatacttcttctccaattattacatagagctaagattttgttggactctgttaaccattctcttgggattaggataatgatgtttctaaaatataattttgagttacacttgagttttgaaatgtcaggtattttcttggctttatgccttgcaaatatatgtatatataaagcactaatttctttgtacatgccctaattggttttgttagattaaaatgaattgaccacatgataattataataactatatttatagataaccatgcattagtaatttataaaaaagattgtcataacttttaaaagaatttagtaaatgtttttttattcaaacacaaatctttcatactattcttattactggcatatagattttttaaaatttgtatatatctggaatgtaccttattactccattttatgaattatgataatttttcagttgatttttaaaatgctataaataatgacaatatttttactatggtaaaacatgtataacataagacttacgatttgaataatttttaaatatacgattcagtagcattaaatatattcacattgctgtgcatctgtcaccactatccaactctctttttcattatcccagactgaaactccctacccattaaacaataattcccaattctgtccttcccctaattcccagtaaccactattttattctgtctctatgaatttgaatgttttaggcaacacatataagtgggatcatacaatatttgtccttttgtgtctggcttatttcacctgccatagtgccttatggttcattcatgttgtaacatgtatctaaatttcgtttctttttcagactgaataatacttcattgtatgtatatgacacattttatctattagtccatggagagacacttgaattgtttctactttttggcagttatgagtaatgcttctatgaatatgggtgtacaaatatctgtttgagtccctgctttcagctcttttaggtgtatacacagaagtgggattactggatcatgtagtaattctatgtttaattttttgaggaactaccattctgtgttccacaatggctacaccattttacatttccaccagcaatgcatgaggatttcagtctttccacactcctgccaacacttgttattttctttatttttttcttgttcttttgaatagtagccatcctaataggtgcgaagtatgataatggcagttcttttcagcttctacttttttgtatctgtaaacaggtcatatgcatttttaaaattgtattttattgcatatatttaaggtatacaacataatgttttaatatacacatacagacagtccttcttatctgtgggttccacattctcagattcaatcaaccaaataatactataacgataaaaataatacaaatttctaaaatatagtataaagtatttgcatagcatttacattgtattagacactataagtgatctagagattatttaaagtatacaggaatatgtaggtaggttatatgcaaattttatatcagggacttgagcatctgcagattttgatatctgccaggtgggtcttggaaccaatcccttgtggatactggggaacaactgtacataatgaaatgattaatacagcaaaacagatcaacatatccatcaccttccatagttactgtgcacaggtgagtgcgtgtgtgtgtgtgtgtggtaagagctcctaaaatctactctctttgctaatttccaatatatgatacagtatttttgtaactcttttttgttgtttagattttgcatttaagtgaaataaatagtacagtactttttctatgtctgtttttgttcgcttgccacagtgtcctttagattcatctgttgtcgtaaatgccagtacctccttttttttaaggctgaataatattcatatatatatatatatacacacacacacgcacatatatatacacacatatatacacatatgtatatatgcacacgtgtatatatacacatatatgcacacgtatatatacacatatatgcacacgtatatatacacatatatgcacatgtgtatatatacacatatatgcacacatgtgtatatacacacatatatatacacacatatatatgccacaatttctttatccatttatctgctgatggatacttacattgtttccatatcttggctactgtgaataaggctgtaatcaacatgaaaatgcagatacctgtgcagttactatacattcccagctcaggcatttttgcttggcataggaatacaaagagaaaagacttagataataagttagagtaacttcacagcagatttcctatgtaagaaatgtagttggacttcttagtttatttaaaaatacctacagatgtgatatgttcaactgtttccatatttgatgaaatatgtatttttaataatcgttactttaatatgcaagtctttacaaatctttttacattttcatgtttctttctaagcttaaaatcagatttctttatttgctttgatatggcaatcatcttcctcctcatcccacccatgtaagctaacacaacctgctagatggaaactttgatctgtagtttgtgaaatggtaggattagagctagagccttctgacatcaacataggcacattcctatctcatcaataacatggtataacaaacttttttatagaaatggttgggattgctatgttttggtaaattgaatatcatggttaactaaattccttcagaaacatacacaagaacatgtgaaccaaatatagggtgttaattcttaaagatattggatacatattcttatttaaaagtatttattgatttattgaacataaactaatcaattaattcagcagaaacttaattatctagaaagtttgccatagccattattttgaatatcaattttgatgtacaatcattccaaggtaaagcacatacaacattgaagtcattgtttaaggatacactcattgatggtagatattttctgagtccactttgttaggtatagttcgtctttttctcataaatgtgaaaagcaaccaaagaaaaatgctttaaaaattcttcatgtctgaagaacagctgaatgacattaatattttctttgtcagacttcagttgcttaaaacttaacttgttcatagacaccacctctgtgataatgcaattaagcagatagatgaacattgttacactgatagtactcagtggttactgctaattaatttattcactggctgatttgtaaaggtgttatttttaatgtcactcaataagatgtgaccctagtcatacaaagagaatgactctcttgatataatcttttaaaaactggaaataaattttgcagaacctgaacaatgtcaaatcaactgtggaaaatgtaggtcctgaaaattagtattattatagcaaagagattatgtttttgttaattttgagtctcaataaccttcccgattgaaggttactataaagtaatacatttacatggtgttagcattttaaccaatatttgtgcctggaattcaaatttgctttttcttatttccgtatgctctgttttaaacatatgttaaattggatatttaaaaatagaagtgcccaagaaacctgcataaatttttttttattatctaaggctttaatgccatcatcattaaaaaaaaaaaaaggaaagagtgactataatttaagtctgtaaaacgacttttaatagcaccaaatagaatgaacagctggcttgctttttgcaaggtgcattgatgttgtcagaagaaactaaataaaaatgctgatacagttgtccaggtttgggttttgccagcacaagaatattttattcattcagccttctctaagttcaaaagattggtgttgactttgatgaaaagatacaatgatacatatttagaatgaccgatctttcacatttaaatcttaaaaatgtgctgctgcagctggatttttctcagggatgccattcaaacaaaatgttacattgtcatctcatatagcttttctatctgtaatgataggatgttaggtgagagtatgacagttcagcacaatttttattatggcattgtactgatacatgaacactaggaccttttttgtttgtttcagtcaacaaagatgtattttcaagtgaaaatattttgaaatagaattttagttgtttcacttaaaaactatagcagagattaaatttaaaggaagtaagctttgtgaacactagattttttgtagttgcagttttctgtactttcatgttcctaatgctaaaaaatctctgtgttctagtcctgccatggagttactaatttttataataatctcttgccaaaagaagaacaactctgataatgcattagagattaagaactttttcattaactttaaaagtgctatagtaattgaaattggtttatccagttatgtttcatttctatttatttatttattttagacacagggttttgctctatcacccaggctgtagtgcagtggtgtgatcatagctcactgcagcctcaaacttctggtttcaagcagtcctcctgcctaggcctcccaaagttctggaattacaggcttgagtcactttgcacctggcctttttcgttattttaaagaatgtttttaaccttttaaaatttccaaatctttacttgttgatgaagtacccagtatttaaacctttacacttgtcagcaaatgctatcaattttgagagcattttaaaaatatgtatttgggcatggacataaagatagaaataatagacactgaggactccaaaagaagagagggagagaggagggaaaatgttggaaaaactacctgttggctactatgttcactatttggtgatgggttcactggaagcccaaaccccagcattatgcaatacacccatataacgaacctgcacatgtactccccaaatctacaattaaaacaatatatgtgtgtgtttttggaaatcttcatgaattgtgctttattttcttttccaaatttaataatgttaataattcataaatacagaaggattcctccctgatgcataacttggtgtctggagttgtatcaacacacatagagacagcgcatgtgctctgccacttgtgtgaagaaaaaccatgcttcttctacaaatatttatttatttgacagggaataacagaggcataaaggaaaataaatacctatgaagtcagttaactgtgcactgatttaaacactttcactacttcattatatgtgtacatgtgttaatgaccacttttgcttgtcaaattgtgtgttgtatttcgatctaaggctcacaatggccttagaatacatatttcatccccatcttttctgtctcttggtagcttcagcatgaagcagaccccacttcctaccatctgctttctccttggaactgacaccaaggtcagctcctctccgatgcctacttgtttcctggtttagaacacttgcatcctgtagttcagccacttccctattaattgatgggccacacatacaggaagcagaacatctaggcctacttgaatattcatgtgactaaataagccaggaagacttttgcctgtggaatgtttaccactattgtttccttacaagtcagttagcaaaaactgaaatatcattttatagttttgtgtttgtatttttagacaagtcgaccattgtattttgttttatggcttgctagtcattgtgttattgcactgaaactttcaatattcatacagatgcaatatcaattatatgctttagatagctattgactacctaaatcaaatttaatatttcaagcccagtagtgatcattcattcagctaatatttttaagcactcatcctataggatgagcactgttttaagcacttgttaaatgatagtgaaaaagaaacatggagcttacagtctaggaaagaagatggatgttaaacaaattagatttgtttgctaaaaaatattagaagtgtaaacactgaatcaactttgctgttatccaaagtctaattatattatttgatttatctaagataatacattaattatattctttttgttttagaaagccattttggcttagaatataattttttgcagctaaccttgtatttgtatctctgtctctatatatctgtgttatctatgtatctacacgagctctggacccaatatttgacttgtaggtagacctaaatttcagtttcatgttcttggatttatgttgagttattacatgtatgtcttcagcattctttagttctctggatcaagaagcaaactgttctttttacataaagctaacttaatacatattagtttttagtcaattagtattttgaaacactatcataattatgtctctacttatttgaatgtatctatatgcttgtatgtgttaaatgtttaacctttgaaaggttacatttttcattttagggattaattatatctctagagatggtttaatatttccataacttttttaaagacttactcatgttagttaacataagttgtttagactaggagtgcctgcattcattaaggacaaaattgatgtgtgtttagtttctttacaaactgtgagcaaggaatcaccattaaatgccattgtatattcattgatcagtgaaatcacatctgggtcacagtggcatctatgtttacagtataaatccctgtggctatgaatgaaaggcttgtttagacttgcatctgcacatagaagtagggatttcatgctgttatcagcctaattttagcctatagaatttcaagtttgctagaggttttgctctccatggtataagtttagcaagaaaagtcatttgtctgctgctctagcagtttagaatgtggaagtatagtgtgcagagttttaatccgtatatgttattaaaacatatacatcattttatatcatacatctgtaataaatattcaaaattaaatagtgatttgggatttattacatcttattactagatgtaataaatgacctcagtgattgtttaaaattgtttttctcaaatataataaaaatacctaaggcataaatcgattgtccaaaaattgaatatatatacacacctcttccattagaactaaatatgtgcaatgtgttcactaaacacttgctgtggtgaaaaacaaaccaaccaaccaaccaacaaacaaacaaaaacacaacatcacagtaactaagtttccagttaaagatttaggcatatttttcataaaaaatttttattcgtaagctttcagaaagtaatccataattgcccttcaaaataaagcttcaaaaactatacatttctacaaagtcttatccataaaacatcaattcagaaacttaaagtgaagttttataatatttgtgctaaatcagttagtctcaaattgtataggaatttagctgtttggcaaaatgacccaaagagctatggacatttttaagtagagacagctaatgaaaatctatatgaatccagtggaggcttttagttattaattttttgactaggacagtatacattgctttagagtcctaaaagtgaggatttaattcttagaaacctaacttaactaggaataaatcaatgtatcactagagccatatacattaggaaaatctcaataaatgtttttcaatgttgatatataaacattcagaatttctctttaaatgtgccaaaagcaacttgataaggcaaatgaattttcttaatgcctaattttctaagaagaaatacatgaattgcatgtaaactttttgctatgtggtgttaacaagtgaggttaaaaaattttaagtctagaaatactgtagcaaaaaattactattttcatgctagcattcttccactctaagcaaactaatttatactgtttcccacctgatagtgatgtaatatgcctttcatttggatttttttctaaatgcatgattcaaatatagattttgtttgcaattagccattatttgacatgaagattgtagtttggattctttagtctgatattgtctgatgggtattttttgcttgttttgtgttgtacatgtgaatcttaagaatttgaaatataagtcacttatcttaactccatctgcagtaaaagagatgagaatggatccagatgttaattttgtataattagtaatgtgctttagaaaaagatttataggtgactgctaccaggctttgtatagtaagatctctgccttctgtttgaccacatcttctactttctgcattgctcactgtgttcctgccacattatctgtctgccttttccacagatatgacaaagatgttttccccacagcctttgcccttgctggtttctctactacctatatactttttataaaattgcaacttctccagcactcatccccaacacacgtatatactgatattctgtgccctcttactctgattcagatttgccttttaattttggcatagattattcataggtggtgctatgtactttacattgcatcacataagatgttacatgtttaatgataccaagattgatcagaggaggcaggtagtaaaagcctgatccatcatattgattgctgtatatttttgacaagaagccattcgtctttgattaatttctttcattttggcataatatgtcccagacacatcgtttgcattttctgttccaaaactggaatcaatgattcaactctaaggagtcctagatttgtttttagtagataatggtatttagagaaaacattttgagtactaggggtgctaatttttactgtcattgcttttaggcctttccagcaggcagagctaggaaattcagtagttttgggaaaaaaatggtgagttaatagtatatttataattcccacttagcattaaaggattttactgaatatttttgaatttatatttacatctgttttctccaacatgaaaaatcttagtcttaacaatattaatgtaattacttgtctgccctatcattttttatatatatatatatatatatatatatatatatatatatatatatatatatatataaaatatgtattaggattctctagagggacagaactaataggatagatgtatatataaaagggagtttattaaggagtattgactcacacgatcacaaggtgaggtcccacaataggccatctgcaaactgaggagcgaagaagccagcccaagtcccaaaacctcaaaagtggggaagctgacagtgcagccttagtctgtggtcaaaggtccaagagtccaaaagctgaagaactcagagtctgatgttcgagggcaggaagtatccagcatgggagaaagatataggccagaagactaagccagactagtctttccatgtccttctacctctgaccatgctggtagctgattagatggtgacaacccagattgagagtaggtctgtctttccaagtccactgactcaaatgttaatctcctttggcaacacctttgaagacacaccctggaacaatactttgcatccttcaatccaatcaagttgacattcagtattaaccatcacattatataactatgaattaataacaaatagtaaaattaagactggtgaattaagtgaaacatttctttgcttctccatttatccttcgttcatattctaatgttgctatacaggcaaaatactgcgttaagtcacttgaaatatttattttctgtatgtggtcttgtgtccagtttatttgttccagttcttaagtttactgtatttgatttctatttgccatatccattcttttttcttctgttcctttcttcctcttttagattaacaatttttttcttctatgttctatctcctctgttatctttttagccatttctctatcttgtttttcattgcttgctcaggaaataacaatatactttaactttcagtataacatcaaataatattaaagtagttcaagaacaatatgcgaacattgcagtggtataatttccatttttttctttctgttccttatgctcttgtcatgtattctactactacatatgttatgaattcaatatgttattatttattcctttaagtaatagtgtcataaaaatttattttctttatttatattacttgtgtatttagtcacatatttactattctgtttctctatttcttcctgcaaacctgagcttccatccccagtgtcttttttcttctgtctcaagaacttttcgcacttgttaaaagctattgtggataaatgatctcatattttgtttgtctgagaatgtctttagttttaaagggtattttctctgaatggagacatcttaattgatatttttccccatttactattttagagtgttccatagcctcgtgtccttcattgttttttaataaaaagtcaactgtcgtgcttatcaccattccctctttatatctcattgtggggcagggtaaggggaaagagttatgctttcatgacattcttggttttcagaagtttaactctatattccctgggtttctatgtatttatccttctcatggttaactgagtttctttgatctgtaagtttgtgtcttctatcaattctaggaagttctgagcatttatttttcaagtagttttttagccccattttttgtcctctccttatgggacacttattacttatatgacaaaccattggatattgtctcatagggctcagattctccttagttattttcttctttattttttaatttgaacaatatctattctcctaccttcaaattcagtgagtgtttcttctgctgtgtccgttctactttaacaccaatcaaatacattttttatttcggatattgtatattttctctttagttttggtatcctattattacagtttcttatttatagtttccatctcttctgatgttcccagtatctttatgcatgttacctgccttttttgccagatcttttattatttttatcatagctatttgaagtccatgtctattaactgcaacatctaaaccacctgtagatttgcctatattatctgtgttttcattgattatgagtcatacttttatgcttctttgtgtgtaccataatttgtaattttatactagatattgggaattaaaaaagagactcgtctggtaacgtttacccttagaaaagcacataccacttcctttgtcctgttgctaatctgtagttaagttttactcttacttatttttagttcactagtagctttcatgaatttgaggataggattaaggatttgccttcagtagagagttatctctatctctcttgctttttccattacagttcatcagcaagcttgtaaagttgcagcggaggtctcttttgtctctaactgccacagtccccttaactggccaaactttctgtaccttgggagacccactttcaaactgattttctgcctgcagacttggactggccaatgacttgcaatctgagagggccccatgagttttggtgggatacttctcagttctgcttccaggtttcagcaggctgccaccttgtggttggggaaggctctgggtccctcagggctatttttttttctttcctcttgccatgtcccaaaccttcagtgggctgcttctattccctttgagaaggctccatgtgcctttggaggaggaaatatttctctaccctcttgcctttctcccagccttcagttgactacttcttggctctcaaagaaagcctcagatactgtggtaggacctctccacaccagctttcctgccatgttctatgcttttggcaggcctccgccttgcacttagtgaagtttattttaatttgttgggatttctttttattgtgtaactgtttttcaaaagtcaaacatttacatgtttgtatttaatagcatataacaagatatattgaaatttatctcatttacattcttattcagtctactctgtacctctcacctgttgctaaccaattttattttcaaacgaattatttttttctttccctttaaaaattaagcatatgttaatgccagtgtatctttcttttttgtaatataaaagatgctatagtgtgtatactatatgctccttcattttaacccccttcataaatcctgttcatcactccacaatggtgtatagaaattttcctcattctttctaacagatgtatattatttatagcattccattttctatctgtatcttaatttattcgacttttctcttcctggtggacatttggatcattttcaatttcttgttaacaccagtaatgctacaatattcataaattgctttctgtttttttattgctttaaggtcatattatgagaaatttttttactattgatttaagatctatgaaatgatctgtattctaattgagattagaaagcagaaaacaatttctacattatctactatgtgcctcacaaaataaatgttaagcaactaaaaaggtatatagaattctgtcctgattatatctatatctatttattaatacctttggggcatctacctcccattttgacttattaggaagcacaaaagaaggtatttccaacaaagatttttatagatatctatatctacatctagatatgtatcttacttggaattaataatttagaagcatctatttcatcttatgaggtgtggtttaggtgtaccattaactgccaactgtatgcctgaatttcaacaaatatttaaaaatgggtatttgcaaaataaatattaaccatatgtatagagtcactgaaattttgcaggtgccatgacagattaaagatactgaataaaaaacatttaaaggtaaatttttcagccaaactcagaacttctcttcatgacatctactgaatatccatatggtgcgtggtctctactgtaaaattggacatttataggttgagcttccattagaaaagagtatgtggaatttatatttcagctcaaaattttgcagttttatttagagcaaaagatagcacagattctttatgtatatatatatatatagagagagagagagagagagagagagagagagagggagagagacagagacagagacagagacagagagagaaagatgtgatactacatgccttttcttcagttctgtatctccatttgctacagaaaatctctactcaaaatggctattgtgttctcaagtctctccttatatcagtgatttcctggacctcttgatttgtctctgtgtctatgcgtgtggatgctttgtatacaattttaaatttgaaacactctttactgtcgtacacaacatgaaggtttagtaacctttttttagaagtgatttataaataaatcagctaactgtgtttgaaaagtctaaactaaagtgcagttgtttgcttcagcttgtcaatggatatttatgaggtgagcataatactataacagttatagcagtgaaatacatcctgtgcagatagctagtaaaggagctcacaagcgttaatgccagtggaattttaatgctcaagagatgggagacatgacacttgctctggttaatgctgctattaccggccatggtgaaaatgagacagtgaagtcagttttaaatattgcagtggagttggggtctctatacctaggcaacagcaaatatgtcagttcctactgttgtcatattttcacctcttggtttatttaactgcagagtagtaatgtaatatcagacatttttcttcagggcatctccattcccttcttacttattatgaggctcatgagaaggttttgctagcaacaatttctgggaaaatgtcaataagcttcccactctaagatatactaatcattgcctgaaaattatttgagtgaagttaaagattccgattgtgagtttagcctttattatgaccagacttttgtttctgtcatatccttggtacattctgtatcttcttatacagaagaaaagtgtgtaatagaataaagtttccacacagagctgtgctctttgtgatgaattgaaactgttaatacattccaatttgtaaagaattatattgtgcttacttttgcctagcaggcaaagaaaatgaagttttcagtcttcagacagatttgttatcatagaagaactttgggattatgtgaagaatagtatggtttcaccatgcttgcatgtataaacttcaagttcatattaatttttcaacttagttcaggttaagttcaaattaatgataatttgacatcatgaggtatctacagagtacctcattaagccctaagagaataagaaaaagcaacacagaaagcatatggtctttttgcataaggtacaggtttaggcaattctttattggaggattgtagaggttaagaggggttcagatattctccgtgactgagaaagttaagaatcaagaaagaaacctttattagtttacatttgcttatacctagtctacttctaaagaagtattgagaaaaaaaatagtcaaacctcagaagaacaaaaattgggaaataataataagccgaagtcaagatatggttataacagaaaatgtacactgaggctagcaattgcttttaggacatcttataaatctgagcttcctaaaagccaagacataaagagaaagacgttaagttgtatagttgccattatataataaaagggaaacatgagcttaccagtattgacaaactttttcctagctctgaacaccaaggagaattggtcccttaatatcagtatccttaatagcaattttccaaaatgataattctcctcatatgaccaaaattcacagggctcttcatataaccaaaaaaaaagagagaatgctatattgtttatgtagatcgacgagaagatctaaagaaaagaaatcaagtctatatatacagatagtttactagtgatctgacttgacacaggcatgaaaatatctggactattagttatcaaactacatttttagcacagacaaaatcttatactgaattgcaattttaaaagatagataagagtggagcattaggtttgagaggcctgcagttgtgcctgcttgttcccttctcatgcactaccccagccacagtgacctctgagtcaccttgcagaaaagccacaggacttcgtggtttacactatgaaaaaaccattgacctagagaaataaatggctagcattccacttagagtcaactgggagtagttttgcaaactgatagggagagaaaggattctaacattattacaggtaaatacctgaggtataaatattctatattgttaattgcatagagaaattatatgccctagatattgggtatgtggttaaaagtttgatgtggtatgaaaattaaagagcctctcttatatttatttcaatgtaatgaactccagcatgcacaggacatgaggtaggttgtacatgtgtgctgacatgcacttatagacatacacacaaacacatttttaacaattagatagccagttgtgaccttgccaaatgctttaacaggcattttattgaaacttttataattttgtatgataagaagaaaatgctgatggtggattataaaatttattaataaattttctaccatataaatgtgctcttgagtcaaactgcattacagtgggtacaccaagatagtaattgtaaaatacagcccaaagatgagtaaaagagttaacttatgggcacattcagtgctgaaaagctttggcatagctggaagttggtggtgattccagaaagtgagtagttagtaagttcttgggctgacttttaaaaacataaatatagatgtttaaaaatgattgcatgtttgctgaatgaattcactctttgaaaactaatgttaccttttaagatagcaaatatttgaatatgcagaaaaggtaaaacatagaatgcgagaagtcatgtgctatcccattacctgcagcatgctggtatattgtctattaaagtctttgcataattacaatcatatcatatttttagttaccatttttttcatctttagaaagtttccttattttaatggctgtagaatagagtaaaatagatgaatctgaatttattcaactagtctctgattgttgcacagaaataaatacaaattgagtatatttttgactactataaaaatcctatagtaaatatttgaaatctcagtctagctattttattaggttagattcctaaagtgaaattactgagtcaaagaatgtaagtttttaaagaccattttttatatactttgcttaccatatagtttttgacaattttaacttccactagcaatgcacaagagttcttttcttgccacagaaattcactaactatgtaatgttttcaatataagtttgcataaaacagtattctttagaagggaggcagttttatttctttaaagtcataatttctattaggcaaataaaatgtttccataaaaagatcataatttaatgattgaattattaatatttacatgcacttcttatatgctatacaaattttaagttggtaaacatactcagagtaatcttatttagattaatttgcctaattctttaatatatggagaaaggtcagctacaacataactgatatctgaataattataatttctaaattaatgtactttaaatgtatttcgctatagcataaagtcagtgataaaattatctttattctgccgaaccttaatttttataaatatttaataaattctacttctaaatttttatgtcatgcaaaattaataaatttaacaatgattttattgaatccaaagagtattatattcccaaaccaaaataaggtaatatatttatagttcattttgaactaatgcagacactaatttaattcaattttatattaactaaatgttgttattgttaaagtttatgattcaatgatttataaggcatgatatcaaattcaagttggttgtattgattgcctaattgagtaagtagtgtgggtgctacctaaagcattacaacaacagtatatttagattttactcatgatttacatgcatgattagggcatgattttaaatcaaaaaagttatttagaaagaatatttaattaaattttcagaaaataaagaaaatattttcttcataataataacatatattacatgttatattattctctaaacacttatgccacctgagttccagtttatcgaattactttgtgaaatacaggcataatattatacattcattcattttaactcttgctcatggaaatcttatgttcctctattttggcatattaaaatgttagaatggtttattttagaagctatgtaatcctgtcacctgatactagtattagcctctatatagagaataaaatattaaaattataataaatataggtctatatgtattcttctatatatttaggttacatttaatagtttcttattaaaccatttagttaacttttgttatttttagcactcgtattatttgttataaaagtgaaatagtctcagaaaaagaaacattattaactaataatactttccattctgtacagttctttgatgagttgtatgttcaagctattttatattgctttgcttgctaccttgataacatacctttattgtgtaggcactataagcatgaggatatgacagacttgtaagccttgtcatattccatttcctgcaaataaaaattgctctgcagtgttctggcattgacttcttgctgtaatataaatggcagcccaaacatccatgaccttaaacataattagcgttatcactaatgtgtgttttgtaagctagtattaggtatagttgaaggacgatgctatctgatcatggtcacttacaaccgggatgcattgccttgtttttagaattttctggtaagcggatggcagcagccaatgtgaagtagttgcctggaagtaagactcctaatggaatactgaataaaatatttccctttagccaaaagtttccatttttcttgattcttctttcttaccttttcctcttctgtgctactctcatctattttctccgtattttcttctctcctttccactgtttttctccttggtaatcctctttctctccctcagattcatacttattctgtcttctcatttttgacatctggctccaccaccagtggaggtctgtgtgattgtgtactcattcattcttcatccatccattcacttgttcctaagtaagaaattattgagtctgctctatgttagacttatgcaatgacattatagagggaatatggataacaacaatatctgttttttttgtttttgttttctttttttttttcttttttttctttttttttttttttgagacggagtctcgctctgtcactaggctggagtgcagtggtgcgatcttggttcactgcaacctccgcctcccgggttcaagcgattctcctgcctcagcctctggagtagctgggactacaggcatgcgctaccacgcccagctaatttttatattttcagtagagatggagtttcaccatgttggccaggatggtcttgatctcttgacctcatgatctgcctgcctcagcctcccaaagtgctgggattacaggcgtgagccaccacacccggccaacaatatgtgtttttattgagagtttgctatatgtcaggcactattttcagtgctttatatatcttaacaaatttagtctttacaacagccttaagaataggcccattttttagtcttcattttactgataaaagaactgaggtacacagtagttaagtaagtcgcccatggtcacacagttaagaagtggcagaggctagaagacatactccatctttagagtgctcagatttaaccactgttatctttccttttagatatttgagcatgctattatggcacgatgtagtgtggctatagtggagatgcagtgggatctttgggagaaggagccccacaactgggaaaactgggaaatgcttaacagtgactcttgaacagatttcccgaagtcacctccttttaaatttttgaaaaataaaattacacccagacattatcagctgttgataaaagtaaaaagcactgagtccatgattagaaggggagaagagcatttgcttgtttttgttaacgggtgtgtgttaaaacatataggtgtatattgcatttaataggtttttctttatttttttttttttggtgcttatatctagagacatttggcagctgataattgatacttaataccaagctttggaagagttgtaataaggacctcagagtcaaaatcagcagagttttcttccttcaagtttccatatctttaattaaaccacatcaaatgaaataaataatttaggtctggaataatttaggtattgaagtgaatttgagaatttgagtagattgtgaatctcacttgttaaactatccagttgcttaaagttaacactctactaaccattactagtcatcacacactaaagttaatatggtatatagaacggaagtggtcacagaagaatcattatatctatatccatttaataagtgataaaacacaagtgaaaatagaggttcataattattttcaaacttgtacagaaaagagaaaactattactttttggtctacaaatacacaagaaaaagatgtcattaatttatatctttgctgtcataatatcattcctcatatatttgttcaattctggaaaattgccagaagataatattttaaatgttgatcactgttgaatttgcatagttcaaatctatttctcattgaatatatatactactgtgttttagaagcactactttttttgcctcaattatttatgtatgatgtcattttcctacctgtccctcttgaaaaagtatttctaaccatggaacataacgtgttgtgaaaaataacacttttattgttttattgtgttttggcaacacacatagctcccttagctaatctagagaagagcacctcttcacttaatatgcactctgaaaatcttgtttgaaagttctaggtaacacaaaaatacagtctggagtacatgacctatgacctgtatcagtaaggaattttgacctcattttaatctgatcttctatctgcttttttcctacttccttttgcactgatatgatttgtagacattaacacaaagtaagtaataaaaattggcaattgtagtaataatgttgcttccaaaagtatttaaaagacagataatttgatgaatgtttgccttttagcaaaaactaatggaaactcctagctctgttttgtttttatgcttttctgtttcttcactaatttctgaaattttcaaatttattgagcagtcatttttggaaaaaatggaaaaaaattctgcttttatattccaaccattcttcttcaacaatgttttgttgcttttgaattttatcatgctttggatattaaaaacaaacttacatttagagggaaataaagagacactctaaaatctaaaacactttgagagcagctattgtaagaaatacagaaatgtcaatagggtgataatcctgaccctaacttagaaagcactatgctagcttttggctggttgaggaatctgggaatgatttaggtggtacagtggatcagtagtatcataccaaagagagaaaaggaaagtatcttttatcactaattggaaactttgttttcaaatatttcttggattatttacaatacacatggaacagtgtgtccacacaaccttcacagaagtgttaagaaattagaatggcaaatcctctttagaaatgtgtaagtgcagatagtcaccatttgaaatgaacacatcatggtggtgtgcagtggtgagaaatggtcagtaacaatggacatgattttgcaaaaatccttcataaaagggaattacaaagagatgacaataccttttttgggattcctcatcctcagtctcttactgcttactaaatgtgatcctcatgtatgcattttaggagcatcagcttgcataagaaaatattttcattttaattcattacataaagaagtatattgggttgtggctttgatatttttatggccgtgatagtaatgctgtattttagataatagtaacattctatttccataattgtttttccatattaattataccataccatctcataggtgaacttactgtggaaaagatctgaatgaaccatcatgctttaacccaagaaaaaaaaattatgacttcatgtctcaagttaacatggtactttccttgggtaatcatttattatcatggtaggaaaactagatgttctcatgcttttttaaataatggcttcaatatttcaatatggatttatttctttcttcagggccggtgcataaacttctcccgagttccatctcagtaaaagggaagcaggaagaccaagaaggtacgaagatggcacattttcacatagctgattttcaaccaaatgaaaaaaatcaagtgcatttcagaagcttttggaagagcagcttaattcctctcagtcgggaaatgttttctctgccttctgctttgcttgcaccaaacatttctaaacacttgttctgccatctacatgggaggtgatgaaactcagtggtaactcatgatttatgacattgaaaataaagaggaacattgacctgcagactatggtttgtacaagaaagtttgtttgaatgtgtagaagaggaaaaagcaacaacagcaacaacacgaagatgataccaaaacaaggaccacaaaacaactagccatgatgggagacaggagttttttacatggaaacatggcacttgtgtttttatgtggcaagatctttatccataggcagagtatgaaatttcccaccaggctaagcaaataaagaagtccattgccttatagctatgtcagatcacagaatccttccaagtgctctatcacagtgtgccttatgggaagtttctgactggaaaatcttgtcattctaacactgaaaagtgcacacgcatgacaaaatgtagacaagatgcctcaaggtattggtagcaagcaagattttgccctttagttttcgaagacacctttctttcattatgcactcgggacaagaaaattaatagagcgttattccacagaaggcctctagccagagatcttgagtgtagtgcaagggactcattttttgcgaacttgtccctgtgactagtagattcccccttttcctgtgtttaggatttagtagtgcataaagcattaatatccataaacatacctagaagtttgttttgcttttaatttaaaggaagcagtaaccacaaagcttccgctcagggttttttctttcttcaagtctccaagggctcttcagcgtcacaagccagcaactctctttgcattaaaatttcaaagtttaattaatataattaaaagcaacagcaagcagcagcctgtgaagattttgctcatcttttttatgccttttgacattgaatgacctattactgtatgcgcattacttggattttgaggggcactctaccttggttatgattcagtagaggaaaaagaccacctttcttcaatttacaaattaaatcttctggagggtcgctatcacaaaacattgacgatgtatgtattataattttttagaaaaaccaccatcgtgtcacgtcgacgatgccaaattatgttagcgtgagcagaaacaccgtgggggaggaaggcagcagctgaagaaaaaagctcaaatgatctagtcactttcgatactgtacttcagatgcgaaatggatattcgagtggaaacctgacaaagtgcgcctgctttgatgtgaactggtatagacaatgaccagtggctgggtcagtgggatgtctctctgtgagcacaaaggcttatcaaatgacactaaaaataagttcaacaaccatcacattggaagggagaaggcgaacatttcatgtttggcgggcatgtgagtgcacaagatggaaagagcgattggagcatcctggtataattacccccattgtgctcttaatggaaatttcaaaggacgggagtattctgttggttggtgtccaggtttgtggcactgttccaagaggccttacacacacacacaaatatataattttctatacatatatatcctctagcttgaaacttttgctcaagtttatttatgtcactggctggctggatccaaagtcatgtgtccacacattcataaataaaaattttacctatg

SEQ ID NO: 2 - DNA, 1464 bp, Homo sapiens
atggtggcggagcggtccccggcccgcagccccgggagctggctgttccccgggctgtggctgttggtgctcagcggtcccggggggctgctgcgcgcccaggagcagccctcctgcagaagagcctttgatctctacttcgtcctggacaagtctgggagtgtggcaaataactggattgaaatttataatttcgtacagcaacttgcggagagatttgtgagccctgaaatgagattatctttcattgtgttttcttctcaagcaactattattttgccattaactggagacagaggcaaaatcagtaaaggcttggaggatttaaaacgtgttagtccagtaggagagacatatatccatgaaggactaaagctagcgaatgaacaaattcagaaagcaggaggcttgaaaacctccagtatcataattgctctgacagatggcaagttggacggtctggtgccatcatatgcagagaaagaggcaaagatatccaggtcacttggggctagtgtttattgtgttggtgtccttgattttgaacaagcacagcttgaaagaattgctgattccaaggagcaagttttccctgtcaaaggtggatttcaggctcttaaaggaataattaattctatactagctcagtcatgtactgaaatcctagaattgcagccctcaagtgtctgtgtgggggaggaatttcagattgtcttaagtggaagaggattcatgctgggcagtcggaatggcagtgttctctgcacttacactgtaaatgaaacatatacaacgagtgtaaaaccagtaagtgtacagcttaattctatgctttgtcctgcacctatcctgaataaagctggagagactcttgatgtttcagtgagctttaatggaggaaaatctgtcatttcaggatcattaattgtcacagccacagaatgttctaacgggatcgcagccatcattgttattttggtgttactgctactcctggggatcggtttgatgtggtggttttggcccctttgctgcaaagtggtgattaaggatcctccaccaccacccgcccctgcaccaaaagaggaggaagaagaacctttgcctactaaaaagtggccaactgtggatgcttcctattatggtggtcgaggggttggaggaattaaaagaatggaggttcgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaagggtcgtcttgatgctctctgggctttgttgaggcggcagtatgaccgggtttctttgatgcgacctcaggaaggagatgagggccggtgcataaacttctcccgagttccatctcag

SEQ ID NO: 3 - protein, 488 aa, Homo sapiens
MVAERSPARSPGSWLFPGLWLLVLSGPGGLLRAQEQPSCRRAFDLYFDLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSILAQSCTEILELQPSSVCVGEEFQIVLSGRGFMLGSRNGSVLCTYTVNETYTTSVKPVSVQLNSMLCPAPILNKAGETLDVSVSFNGGKSVISGSLIVTATECSNGIAAIIVILVLLLLLGIGLMWWFWPLCCKVVIKDPPPPPAPAPKEEEEEPLPTKKWPTVDASYYGGRGVGGIKRMEVRWGDKGSTEEGARLEKAKNAVVKIPEETEEPIRPRPPRPKPTHQPPQTKWYTPIKGRLDALWALLRRQYDRVSLMRPQEGDEGRCINFSRVPSQ

SEQ ID NO: 4 - DNA, 1343 bp, Homo sapiens
cgctgccgcaggcgccggcgtctcagctgctcgccgccccccaccccagagtgcgtgcagggtgactcccgccacctttgcgaccctcctgagcttaggggactgcgagcgggagggagtctcaggcccccggccgcaggatggtggcggagcggtccccggcccgcagccccgggagctggctgttccccgggctgtggctgttggtgctcagcggtcccggggggctgctgcgcgcccaggagcagccctcctgcagaagagcctttgatctctacttcgtcctggacaagtctgggagtgtggcaaataactggattgaaatttataatttcgtacagcaacttgcggagagatttgtgagccctgaaatgagattatctttcattgtgttttcttctcaagcaactattattttgccattaactggagacagaggcaaaatcagtaaaggcttggaggatttaaaacgtgttagtccagtaggagagacatatatccatgaaggactaaagctagcgaatgaacaaattcagaaagcaggaggcttgaaaacctccagtatcataattgctctgacagatggcaagttggacggtctggtgccatcatatgcagagaaagaggcaaagatatccaggtcacttggggctagtgtttattgtgttggtgtccttgattttgaacaagcacagcttgaaagaattgctgattccaaggagcaagttttccctgtcaaaggtggatttcaggctcttaaaggaataattaattcttctaacgggatcgcagccatcattgttattttggtgttactgctactcctggggatcggtttgatgtggtggttttggcccctttgctgcaaagtggttattaaggatcctccaccaccacccccccctgcaccaaaagaggaggaagaagaacctttgcctactaaaaagtggccaactgtggatgcttcctattatggtggtcgaggggttggaggaattaaaagaatggaggttcgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaagggtcgtcttgatgctctctgggctttgttgaggcggcagtatgaccgggtttctttgatgcgacctcaggaaggagatgaggtttgtatatgggaatgtattgagaaagagctaactgcttgagtcagtataatggaggcagggaaatagtaataaaaaatgatt

SEQ ID NO: 5 - protein, 386 aa, Homo sapiens [sequence omitted]

SEQ ID NO: 6 - protein, 489 aa, Homo sapiens
MVAERSPARSPGSWLFPGLWLLVLSGPGGLLRAQEQPSCRRAFDLYFDLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSILAQSCTEILELQPSSVCVGEEFQIVLSGRGFMLGSRNGSVLCTYTVNETYTTSVKPVSVQLNSMLCPAPILNKAGETLDVSVSFNGGKSVISGSLIVTATECSNGIAAIIVILVLLLLLGIGLMWWFWPLCCKVVIKDPPPPPAPAPKEEEEEPLPTKKWPTVDASYYGGRGVGGIKRMEVRWGDKGSTEEGARLEKAKNAVVKIPEETEEPIRPRPPRPKPTHQPPQTKWYTPIKGRLDALWALLRRQYDRVSLMRPQEGDEVCIWECIEKELTA

SEQ ID NO: 7 - protein, 322 aa, Homo sapiens
MVAERSPARSPGSWLFPGLWLLVLSGPGGLLRAQEQPSCRRAFDLYFDLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSILAQSCTEILELQPSSVCVGEEFQIVLSGRGFMLGSRNGSVLCTYTVNETYTTSVKPVSVQLNSMLCPAPILNKAGEWGLTVTQAGVKWHLDTHCTFGLSGSGDPPTSAS

SEQ ID NO: 8 - protein, 368 aa, Homo sapiens
MVAERSPARSPGSWLFPGLWLLVLSGPGGLLRAQEQPSCRRAFDLYFDLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSILAQSCTEILELQPSSVCVGEEFQIVLSGRGFMLGSRNGSVLCTYTVNETYTTSVKPVSVQLNSMLCPAPILNKAGETLDVSVSFNGGKSVISGSLIVTATECSNGIAAIIVILVLLLLLGIGLMWWFWPLCCKVVIKDPPPPPCTKRGGRRTFAY

SEQ ID NO: 9 - DNA, 153 bp, Homo sapiens
atggtggcggagcggtccccggcccgcagccccgggagctggctgttccccgggctgtggctgttggtgctcagcggtcccggggggctgctgcgcgcccaggagcagccctcctgcagaagagcctttgatctctacttcgtcctggacaag

SEQ ID NO: 10 - DNA, 69 bp, Homo sapiens
tctgggagtgtggcaaataactggattgaaatttataatttcgtacagcaacttgcggagagatttgtg

SEQ ID NO: 11 - DNA, 75 bp, Homo sapiens
agccctgaaatgagattatctttcattgtgttttcttctcaagcaactattattttgccattaactggagacagg

SEQ ID NO: 12 - DNA, 79 bp, Homo sapiens
ggcaaaatcagtaaaggcttggaggatttaaaacgtgttagtccagtaggagagacatatatccatgaaggactaaagc

SEQ ID NO: 13 - DNA, 108 bp, Homo sapiens
gcgaatgaacaaattcagaaagcaggaggcttgaaaacctccagtatcataattgctctgacagatggcaagttggacggtctggtgccatcatatgcagagaaagag

SEQ ID NO: 14 - DNA, 69 bp, Homo sapiens
gcaaagatatccaggtcacttggggctagtgtttattgtgttggtgtccttgattttgaacaagcacag

SEQ ID NO: 15 - DNA, 81 bp, Homo sapiens
cttgaaagaattgctgattccaaggagcaagttttccctgtcaaaggtggatttcaggctcttaaaggaataattaattct

SEQ ID NO: 16 - DNA, 60 bp, Homo sapiens
atactagctcagtcatgtactgaaatcctagaattgcagccctcaagtgtctgtgtgggg

SEQ ID NO: 17 - DNA, 102 bp, Homo sapiens
gaggaatttcagattgtcttaagtggaagaggattcatgctgggcagtcggaatggcagtgttctctgcacttacactgtaaatgaaacatatacaacgagt

SEQ ID NO: 18 - DNA, 69 bp, Homo sapiens
gtaaaaccagtaagtgtacagcttaattctatgctttgtcctgcacctatcctgaataaagctggagag

SEQ ID NO: 19 - DNA, 78 bp, Homo sapiens
actcttgatgtttcagtgagctttaatggaggaaaatctgtcatttcaggatcattaattgtcacagccacagaatgt

SEQ ID NO: 20 - DNA, 98 bp, Homo sapiens
tctaacgggatcgcagccatcattgttattttggtgttactgctactcctggggatcggtttgatgtggtggttttggcccctttgctgcaaagtggt

SEQ ID NO: 21 - DNA, 42 bp, Homo sapiens
attaaggatcctccaccaccacccgcccctgcaccaaaagag

SEQ ID NO: 22 - DNA, 96 bp, Homo sapiens
gaggaagaagaacctttgcctactaaaaagtggccaactgtggatgcttcctattatggtggtcgaggggttggaggaattaaaagaatggaggtt

SEQ ID NO: 23 - DNA, 165 bp, Homo sapiens
cgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaag

SEQ ID NO: 24 - DNA, 81 bp, Homo sapiens
ggtcgtcttgatgctctctgggctttgttgaggcggcagtatgaccgggtttctttgatgcgacctcaggaaggagatgag

SEQ ID NO: 25 - DNA, 36 bp, Homo sapiens
ggccggtgcataaacttctcccgagttccatctcag

SEQ ID NO: 26 - DNA, 18 bp, Artificial sequence
  Primer
agagtgcgtgccgggtga

SEQ ID NO: 27 - DNA, 24 bp, Artificial sequence
  Primer
gaaagaagacagcaacagggcacc

SEQ ID NO: 28 - DNA, 21 bp, Artificial sequence
  Primer
gacggagtcttgctctgggac

SEQ ID NO: 29 - DNA, 21 bp, Artificial sequence
  Primer
gtgcaatacgaccttgaggca

SEQ ID NO: 30 - DNA, 20 bp, Artificial sequence
  Primer
ctggaccattcagtgagacc

SEQ ID NO: 31 - DNA, 20 bp, Artificial sequences
  Primer
gcctgaatcaccacttggaa

SEQ ID NO: 32 - DNA, 23 bp, Artificial sequence
  Primer
agcttagttacaatactgccatg

SEQ ID NO: 33 - DNA, 21 bp, Artificial sequence
  Primer
ccagtgtcacaatgtcatcag

SEQ ID NO: 34 - DNA, 24 bp, Artificial sequence
  Primer
gccaacttaaaggtactctgactg

SEQ ID NO: 35 - DNA, 24 bp, Artificial sequence
  Primer
tctagataatgaccacctgcactg

SEQ ID NO: 36 - DNA, 23 bp, Artificial sequence
  Primer
gaagtatggagaagacctcaagg

SEQ ID NO: 37 - DNA, 20 bp, Artificial sequence
  Primer
gcctgtcacacaatatgctc

SEQ ID NO: 38 - DNA, 19 bp, Artificial sequence
  Primer
ggaaagccagcacagttgg

SEQ ID NO: 39 - DNA, 20 bp, Artificial sequence
  Primer
tgctgatgtgctttgcagag

SEQ ID NO: 40 - DNA, 21 bp, Artificial sequence
  Primer
tgaactctgattgaagcatgc

SEQ ID NO: 41 - DNA, 18 bp, Artificial sequence
  Primer
ggcttgcccaaggcttac

SEQ ID NO: 42 - DNA, 22 bp, Artificial sequence
  Primer
caggagtttgagacccttactc

SEQ ID NO: 43 - DNA, 27 bp, Artificial sequence
  Primer
ccatagattatttctggatggaattgc

SEQ ID NO: 44 - DNA, 22 bp, Artificial sequence
  Primer
ggaatttgaccataagctgtgc

SEQ ID NO: 45 - DNA, 26 bp, Artificial sequence
  Primer
gaaactttgctgttattaacatggca

SEQ ID NO: 46 - DNA, 21 bp, Artificial sequence
  Primer
gacttctttggagctaccaca

SEQ ID NO: 47 - DNA, 23 bp, Artificial sequence
  Primer
gccctagaaatacatactccaga

SEQ ID NO: 48 - DNA, 24 bp, Artificial sequence
  Primer
ctctgagatgtgaactaaaggacc

SEQ ID NO: 49 - DNA, 21 bp, Artificial sequence
  Primer
gggctgatgcaatgattgtgc

SEQ ID NO: 50 - DNA, 25 bp, Artificial sequence
  Primer
gacttcatgtctcaagttaacatgg

SEQ ID NO: 51 - DNA, 23 bp, Artificial sequence
  Primer
cagaaggcagagaaaacatttcc

SEQ ID NO: 52 - DNA, 20 bp, Artificial sequence
  Primer
gtgagcttttaacttggcca

SEQ ID NO: 53 - DNA, 21 bp, Artificial sequence
  Primer
ccttgtcttccttacaaaccc

SEQ ID NO: 54 - DNA, 20 bp, Artificial sequence
  Primer
cctagccaatagagaccgtg

SEQ ID NO: 55 - DNA, 19 bp, Artificial sequence
  Primer
agagagatccctcatccct

SEQ ID NO: 56 - DNA, 21 bp, Artificial sequence
  Primer
aagctaagacccaacttcttt

SEQ ID NO: 57 - DNA, 17 bp, Artificial sequence
  Primer
tcatgcaatccacacag

SEQ ID NO: 58 - DNA, 25 bp, Artificial sequence
  Primer
aatccagtaaatgaaatagtcatca

SEQ ID NO: 59 - DNA, 20 bp, Artificial sequence
  Primer
ataagccaaacatgatggga

SEQ ID NO: 60 - DNA, 20 bp, Artificial sequence
  Primer
catgtttccactccagttct

SEQ ID NO: 61 - DNA, 20 bp, Artificial sequence
  Primer
ataaagggcagttagggatg

SEQ ID NO: 62 - DNA, 25 bp, Artificial sequence
  Primer
gagcaaaactctgtctcaaaaataa

SEQ ID NO: 63 - DNA, 22 bp, Artificial sequence
  Primer
ggcttacttggaaaggtctctt

SEQ ID NO: 64 - DNA, 20 bp, Artificial sequence
  Primer
gtctcaccctgaaagggatt

SEQ ID NO: 65 - DNA, 20 bp, Artificial sequence
  Primer
ggttacaggaccacaagtgc

SEQ ID NO: 66 - DNA, 20 bp, Artificial sequence
  Primer
tactccagcctggatgacag

SEQ ID NO: 67 - DNA, 20 bp, Artificial sequence
  Primer
tgttccataacaagcacgtt

SEQ ID NO: 68 - protein, 198 aa, homo sapiens
GSHMASRQEQDIVFLIDGSGSISSRNFATMMNFVRAVISQFQRPSTQFSLMQFSNKFQTHFTFEEFRRSSNPLSLLASVHQLQGFTYTATAIQNVVHRLFHASYGARRDAAKILIVITDGKKEGDSLDYKDVIPMADAAGIIRYAIGVGLAFQNRNSWKELNDIASKPSQEHIFKVEDFDALKDIQNQLKEKIFAIEG

(19) United States
(12) Patent Application Publication
(10) Pub. No.: US 2005/0221336 A1
(43) Pub. Date: Oct. 6, 2005

(54) Mutations in capillary morphogenesis gene-2 (CMG-2) and use thereof

(75) Inventors: John Martignetti, New York, NY (US); Oonagh Dowling, Brooklyn, NY (US)

Correspondence Address: DARBY & DARBY P.C., P. O. BOX 5257, NEW YORK, NY, 10150-5257, US

(73) Assignee: Mount Sinai School of Medicine of New York University, New York, NY (US)

(21) Appl. No.: 10/939,321

(22) Filed: Sep. 9, 2004

Related U.S. Application Data
(60) Provisional application No. 60/501,865, filed on Sep. 10, 2003.

(51) Int. Cl. C12Q 1/68

(52) U.S. Cl. 435/6

(57) ABSTRACT

Mutations and polymorphisms in a particular gene, the capillary morphogenesis gene-2 (CMG-2) have been identified. The mutations have been associated with infantile systemic hyalinosis (ISH) and juvenile hyaline fibromatosis (JHF), as well as conditions associated with these disorders. Described herein are variant CMG-2 nucleic acids and variant CMG-2 polypeptides; cells comprising such variant CMG-2 nucleic acids and/or expressing variant CMG-2 polypeptides; and methods of diagnosing and treating such disorders and conditions. Variant CMG-2 proteins include those comprising one or more of E220X, G105D, L329, P257insC, 1189T, A357P, and A322S. Variant CMG-2 nucleic acids include those encoding these mutant CMG-2 proteins, as well as silent mutations or polymorphisms.

This application claims priority from U.S. Provisional Application Ser. No. 60/501,865, filed on Sep. 10, 2003, which is herein incorporated by reference in its entirety.

### FIELD OF THE INVENTION

The present invention relates to certain syndromes and conditions associated with a deficiency in the capillary morphogenesis gene-2 (CMG-2) protein. In particular, the invention relates to diagnostic and therapeutic applications for juvenile hyaline fibromatosis (JHF), infantile systemic hyalinosis (ISH), and conditions associated with these disorders. Applications based on specific mutations and/or polymorphisms in the CMG-2 gene are contemplated.

### BACKGROUND OF THE INVENTION

JHF and ISH are autosomal recessive syndromes of unknown etiology, characterized by multiple recurring subcutaneous tumors, gingival hypertrophy, joint contractures, osteolysis and osteoporosis. Both disorders present in infancy with papulonodular skin lesions, particularly of the perianal, perinasal and perioral areas. Affected individuals often develop several associated features including multiple subcutaneous tumors, gingival hypertrophy, flexion contractures of joints, osteolytic lesions and osteopenia (Landing and Nadorra, Pediat Path 1986;6:55-79; Fayad et al., Am J Med Genet 1987;26:123-131; Keser et al., Clin. Rheumatol. 1999; 18:248-252). ISH has a more severe phenotype than JHF, including an earlier onset, more painful and severe course, and, histologically, by widespread deposition of hyaline material throughout the skin, gastro-intestinal tract, endocrine glands, and muscle (Landing and Nadorra, Pediat Path 1986;6:55-79). In addition, ISH has been associated with an increased susceptibility to bone fractures, infections and death in infancy (Stucki et al., Am J Med Genet 2001;100:122-129). Diagnosis can generally only be based on clinical findings, including distribution of skin lesions, and biopsy which typically reveals the presence of an abundant extracellular, acidophilic hyaline material.

Because of their significant phenotypic overlaps, JHF and ISH have been suggested to be allelic (Mancini et al., Dermatology 1999; 198:18-25). The JHF disease gene has been localized to chromosome 4q21 using a positional cloning approach (Rahman et al., Am J Hum Genet 2002;71:975-980). The 5.3 cM/6.9 Mb locus is bounded by microsatellite marker D4S2393 centromerically and D4S395 telomerically (Rahman et al., Am J Hum Genet 2002;71:975-980 2002; Kong et al., Nat Genet 2002;31:241-7). Recently, several mutations in a gene located in this region, the capillary morphogenesis gene-2 (CMG-2), were identified in families with JHF or HIS (Hanks et al., Am J Med Genet 2003 (published on internet ahead of print)). The CMG-2 gene was originally identified on the basis of its up-regulation in endothelial cells induced to undergo capillary formation (Bell et al., J Cell Sci 2001; 114:2755-2773), and was recently shown to function as an anthrax toxin receptor (Scobie et al., Proc Natl Acad Sci USA 2003;100:5170-5174). However, the physiologic role of the encoded protein, CMG-2, is unknown.

Since the underlying mechanisms of JHF and ISH disorders are still unknown, treatment options alleviating the cause or causes of the disorders are not available. Elucidating the causes and mechanisms of JHF and ISH could also offer new and improved strategies for diagnosis of patients suffering from one or more of the conditions associated with the diseases, e.g., osteoporosis and arthritis, as well as new treatment options for such conditions. Thus, there is a need in the art for improved diagnostic methods and new therapeutic regimens for JHF and ISH, and for the conditions associated with these disorders. The invention addresses these and other needs in the art.

### SUMMARY OF THE INVENTION

Accordingly, the present invention provides a method for diagnosing a disease, disorder, or condition associated with at least one of osteoporosis, osteopenia, osteolysis, and arthritis, in a subject, which method comprises detecting a variant capillary morphogenesis gene-2 (CMG-2) gene in the subject. In one embodiment, the disorder or condition is infantile systemic hyalinosis (ISH) or juvenile hyaline fibromatosis (JHF). The variant may have a mutation or polymorphism which is a member of the group consisting of, for example, a deletion, an insertion, a substitution, and combinations thereof. The mutation or polymorphism may be in a coding or non-coding region of the gene. In one embodiment, the mutation or polymorphism results in a variant of a CMG-2 protein having the sequence of SEQ ID NO:3, 5, 6, or 7. In another embodiment, the mutation or polymorphism results in the deletion of a segment comprising more than one amino acid of SEQ ID NO:3, 5, 6, or 7. The mutation in the CMG-2 protein can be selected from the group consisting of, e.g., (a) E220X, (b) G105D, (c) L329R; (d) P257insC; and (e) I189T. The polymorphism can in the CMG-2 protein can, for example, be selected from A357P and A322S. In one embodiment, the mutation in the CMG-2 gene, having the sequence of SEQ ID NO:1, is selected from the group consisting of: (a) G37632T; (b) G17627A; (c) T65089G; (d) C88794CC; and (e) T19288C. In another embodiment, the polymorphism in the CMG-2 gene, having the sequence of SEQ ID NO:1, is selected from C88790G and C48964T.

The invention also provides for a kit for diagnosing a disease, disorder, or condition associated with at least one of osteoporosis, osteopenia, osteolysis, and arthritis, comprising an oligonucleotide that specifically hybridizes to or adjacent to a site of mutation or polymorphism in a CMG-2 gene, and instructions for use. The disorder or condition may be, for example, JHF or ISH. In one embodiment, the site of mutation or polymorphism comprises a nucleotide selected from the group consisting of nucleotides 37632, 17627, 65098, 88794, 19288, 17700, 18352, 19400, 88790, 116113, 166226, and 48964. The kit may comprise at least one probe comprising the site of mutation or polymorphism. Alternatively, the kit comprises a first oligonucleotide primer comprising at least 15 consecutive nucleotides of SEQ ID NO:1, and a second oligonucleotide primer comprising at least 15 consecutive nucleotides of a sequence complementary to SEQ ID NO:1. The kit may also comprise a first nucleotide sequence selected from the group consisting of SEQ ID NOS:26, 28, 30, 32, 34, 36, 38, 40, 42, 44, 46, 48, and 50, and a second primer selected from the group consisting of SEQ ID NOS: 27, 29, 31, 33, 35, 37, 39, 41, 43, 45, 47, 49, and 51.

The invention also provides for a kit for diagnosing a disease, disorder, or condition associated with at least one of osteoporosis, osteopenia, osteolysis, and arthritis comprising an antibody that specifically recognizes a mutation or polymorphism in a CMG-2 protein; and instructions for use. The disorder or condition can be, for example, JHF or ISH. In one embodiment, the CMG-2 protein has the sequence of SEQ ID NO:3, 5, 6, or 7. The mutation can selected from selected from the group consisting of, e.g., (a) E220X, (b) G105D, (c) L329R; (d) P257insC; and (e) I189T. The polymorphism can in the CMG-2 protein can, for example, be selected from A357P and A322S.

The invention also provides for a method for diagnosing a disease, disorder, or condition associated with at least one of osteoporosis, osteopenia, osteolysis, and arthritis in a subject, which method comprises assessing the level of expression or activity of wild-type CMG-2 having the sequence of SEQ ID NO:3, 5, 6, or 7 in the test subject and comparing it to the level of expression or activity of wild-type CMG-2 in a control subject, wherein a decreased level of expression is indicative of the disease, disorder, or condition. The disorder or condition may, for example, be JHF or ISH. In one embodiment, the level of expression is assessed by determining the amount of mRNA that encodes the wild-type CMG-2 in a biological sample. In another embodiment, the level of expression of is assessed by determining the concentration of wild-type, full-length CMG-2 protein in a biological sample. The level of activity can, for example, be assessed by determining the level of fibroblasts in a biological sample capable of binding to laminin. Alternatively, the level of activity can be assessed by determining the level of fibroblasts in a biological sample capable of producing mature matrix metalloproteinase 2 (MMP-2).

The invention also provides for a method for treating a disease, disorder, or condition associated with at least one of osteoporosis, osteopenia, osteolysis, and arthritis, which method comprises administering to a patient in need of such treatment an effective amount of an agent that provides CMG-2 activity, in association with a pharmaceutically acceptable carrier. The disorder or condition may, for example, be JHF or ISH. In one embodiment, the agent is wild-type CMG-2 protein having the sequence of SEQ ID NO:3, 5, 6, or 7. In another embodiment, the agent is a gene encoding CMG-2 protein having the sequence of SEQ ID NO:2 or 4.

The invention also provides for an isolated variant of CMG-2 having the sequence of SEQ ID NO:3, 5, 6, or 7, the variant comprising a mutation selected from the group consisting of E220X, G105D, L329R; P257insC, and I189T. The invention further provides for an isolated cell comprising a vector, which vector comprises a nucleic acid encoding the CMG-2 variant, operatively associated with an expression control sequence. The cell can be selected from a prokaryotic cell and an eukaryotic cell. The invention additionally provides for an isolated nucleic acid encoding the CMG-2 variant, as well as for an isolated oligonucleotide which specifically hybridizes to the nucleic acid.

The invention also provides for an isolated variant of CMG-2 having the sequence of SEQ ID NO:3, 5, 6, or 7, the variant comprising a polymorphism selected from A357P and A322S. The invention further provides for an isolated cell comprising a vector, which vector comprises a nucleic acid encoding such a CMG-2 variant, operatively associated with an expression control sequence. The cell can be selected from a prokaryotic cell and an eukaryotic cell. The invention additionally provides for an isolated nucleic acid encoding the CMG-2 variant, as well as for an isolated oligonucleotide which specifically hybridizes to the nucleic acid.

The above features and many other advantages of the invention will become better understood by reference to the following detailed description when taken in conjunction with the accompanying drawings.

### BRIEF DESCRIPTION OF THE DRAWINGS

FIG. 1. Predicted amino acid sequence of CMG-2 (SEQ ID NO:3).

FIG. 2A to 2D. Pedigrees and haplotypes analysis in four JHF/ISH families. (A) Family JHF1. (B) Family JHF2. (C) Family ISH1. (D) Family ISH2. Genotypes are represented by allele sizes in base pairs and markers are ordered according to their physical order. Blackened symbols denote affected individuals and shaded areas denote disease segregating haplotypes.

FIG. 3. Predicted CMG-2 protein structure. The protein is 487 amino acids in length and contains an N-terminal signal peptide followed by a von Willebrand factor type A domain, a transmembrane domain and a cytosolic tail. Mutations were identified in exons 3, 7, 8 and 12 and are shown relative to affected protein domains.

FIG. 4. CMG-2 isoforms identified by Scobie et al., 2003, supra. The different isoforms depicted are CMG-2⁴⁸⁹, CMG-2⁴⁸⁸, CMG-2³⁸⁶, and CMG-2³²². CMG-2⁴⁸⁹ has a signal peptide, extracellular VWFA domain and a transmembrane region. CMG-2⁴⁸⁸ is identical to that of CMG-2⁴⁸⁹ except that the last 12 amino acids of the cytoplasmic tail diverge in the two isoforms (this is shown by different shading). CMG-2³⁸⁶ is identical to CMG-2⁴⁸⁹ except its missing the amino acids 213-315. It is expected that this variant is secreted since it has a signal peptide but is missing the transmembrane domain.

FIG. 5A and 5B. DNA sequence analysis of CMG-2 in individuals with ISH and JHF. (A) Three homozygous mutations were identified: GAA>TAA (E220X) nonsense mutation in Exon 8 of ISH1 family; GGC→GAC (G105D) missense mutation in Exon 4 of JHF1 family; and CTA→CGA(L329R) missense mutation in Exon 12 of JHF2 family. (B) Both affected children in family ISH2 were compound heterozygotes: ATT→ACT (I189T) missense mutation (paternal allele) and a nucleotide insertion, P357insC (maternal allele).

FIG. 6A to 6E. Molecular modeling of CMG-2 mutations: (A) Supraposition of CMG-2 model (dark grey) with chain A of the Alpha-X Beta2 Integrin I Domain (light grey; PDB accession number 1N3Y; SEQ ID NO:68). Non-conserved residues were mutated using the software program O (Jones et al., Acta Crystallogr 1991;47:110-119) and the CMG-2 model was minimized using MOE (Molecular Operating Environment) software. The root mean square deviation of the CMG-2 model from the integrin template is about 1.03 Å, with greater variation in the loops and less variance in the conserved regions where the mutations reside. (B and C) Glycine 105 is mutated to an aspartate C, within the extracellular region, and is rendered with SPOCK and Raster3D (Merritt and Bacon, Meth Enzymol 1997;277:505-524). (D and E) Isoleucine 189 is mutated to threonine and contours are provided by the calculated electron density. A cavity is formed as depicted by the purple asterisk (*) in (E).

FIG. 7. CMG-2 mutations result in altered CMG-2 protein expression as detected by Western blotting. 293 cells were transfected with 1.5 mg of plasmid DNA (in 6-well dishes) using Lipofectamine 2000 and various CMG-2 WT and mutant constructs, as indicated. Following transfection, cells were lysed after 24 hr with 0.5 ml SDS-PAGE sample buffer containing mercaptoethanol and treated at 100° C. for 10 minutes. 30 mL of sample was loaded per lane on an 10% SDS-PAGE gel and protein samples were transferred to PVDF membranes and probed with anti-CMG-2 affinity purified antibodies (1 mg/ml) as described (Bell et al., 2001, supra). Closed arrowheads indicate the position of anti-CMG-2 reactive mutant proteins; Solid arrow indicates the position of CMG-2 WT protein observed in 293 cells transfected with pCIneo-CMG-2-WT.

FIG. 8A to 8L. Crystal violet staining of adherent patient and control primary fibroblasts to laminin, collagen I and collagen IV extracellular matrix. Cells were plated in serum free media at a density of 1×10⁵ cells/well and allowed to adhere to laminin, collagen I and collagen IV 24 well plates (BD Biosciences) for 75 min. Unbound cells were removed by washing with PBS and adherent cells were fixed in ethanol (10 min), stained with 0.5% crystal violet (20 min), washed extensively with water, and solubilized with 800 μl 1% SDS. Relative adhesion was quantified by monitoring the absorbance of released dye at 540 nm (n=4). Experiments were repeated three times in quadruplicate. Cells are shown at 7.5 magnification. Bar charts. (A), (B), and (C) are control fibroblasts stained for laminin, collagen I, and collagen IV, respectively. (D), (E), and (F) are JHF1 fibroblasts stained for laminin, collagen I, and collagen IV, respectively. (G), (H), and (I), are ISH2 fibroblasts stained for laminin, collagen I, and collagen IV, respectively. (J), (K), and (L) are bar charts indicating relative adhesion of patient fibroblasts compared to control fibroblasts for laminin, collagen I, and collagen IV, respectively.

FIG. 9A to 9F. Correction of CMG-2 deficient fibroblast laminin binding defect with serum. As shown in this figure, the addition of 5% serum to CMG-2 deficient fibroblasts, derived from both patients with JHF and the more severe ISH, grown in culture corrects their previous inability to bind to laminin. (A), (C), and (E) are controls, i.e., without serum, for control, JHF, and ISH fibroblasts, respectively. (B), (D), and (F) represent control, JHF, and ISH fibroblasts, respectively, incubated with serum.

FIG. 10. Overall upregulation of MMP-2 expression but loss of MMP-2 activation by CMG-2 deficient cells grown on laminin. While normally a rich source of active MMP-2, supernatant from CMG-2 deficient fibroblasts grown in serum-free media shows the presence of the inactive pro-form when assayed by zymography. JHF cells, those derived from individuals with the milder disease, show partial activation. ISH-derived fibroblasts, those with the more severe and fatal disease, have virtually no active MMP-2. Notably, normal fibroblasts produced no to very little amounts of MMP-2 protein under the same conditions, further emphasizing the differences between CMG-2 deficient cells and normal cells.

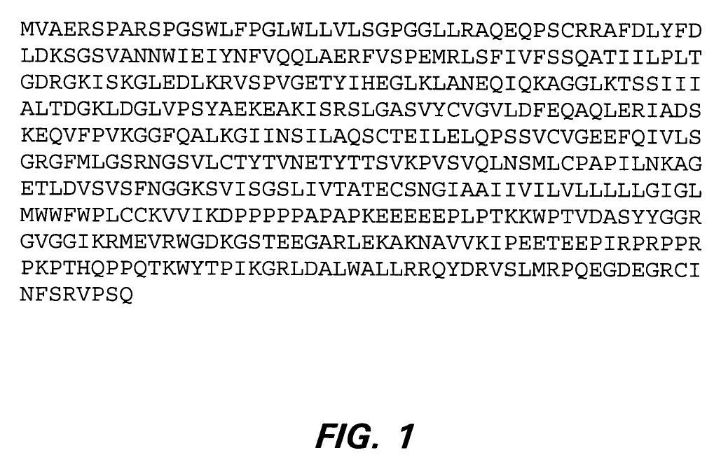

### DETAILED DESCRIPTION OF THE INVENTION

The present invention is, in part, based on the identification of mutations in the capillary morphogenesis gene-2, CMG-2, that associate closely with certain syndromes such as JHF and IHS, as well as with certain conditions associated with a CMG-2 deficiency. CMG-2 mutations, include, but are not limited to, E220X, G105D, L329, P257insC, and 1189T, and other amino acid and nucleotide changes described in Tables 1A and 1B below.

TABLE 1A
CMG-2 Mutations Associated With JHF and ISH

| Mutation | | Mutation | | |
|---|---|---|---|---|
| in CMG-2 | | in CMG-2 | | |
| Genomic DNA | Codon | Protein (SEQ | Predicted | Dis- |
| (SEQ ID NO: 1) | Substitution | ID NO: 3) | Effect | order |
| G37632T | GAA(220)TAA | E220X | Loss of | ISH |
| | in exon 8 | | residues | |
| | | | 220 et seq. | |
| | | | in CMG-2 | |
| G17627A | GGC(105)GAC | G105D | Non- | JHF |
| | in exon 4 | | conservative | |
| | | | mutation | |
| T65089G | CTA(329)CGA | L329R | Non- | JHF |
| | in exon 12 | | conservative | |
| | | | mutation | |
| C88794CC | insert C in | P257insC | Loss of | ISH |
| | exon 13 | | residues | |
| | | | 257 et seq.; | |
| | | | new 12-amino | |
| | | | acid C-termi- | |
| | | | nal (see SEQ | |
| | | | ID NO: 8). | |
| T19288C | T(189)C in | I189T | Possible | ISH |
| | exon 7 | | cavity | |
| | | | formed in | |
| | | | CMG-2 | |

TABLE 1B
Polymorphisms and Non-Coding Mutations in CMG-2 Gene Identified
in non-ISH or non-JHF Individuals. IVS = intervening
sequence. The position of the nucleotide change in Exon 13
and UTR regions refers to the cDNA position.

| Location (genomic | | Predicted |
|---|---|---|
| nucleotide position) | Nucleotide change | Effect |
| Intron 4 (17,700 bp) | IVS4 + 8A→C | — |
| Intron 6 (18,352 bp) | IVS6 + 29A→C | — |
| Intron 7 (19,400 bp) | IVS7 + 54T→A | — |
| Exon 13 (88,790 bp) | 1597C→G | A357P |
| Intron 16 (116,113 bp) | IVS16-47T→A | — |
| 3′ UTR (166,226 bp) | 2023C→T | — |
| Nucleotide position | CMG-2⁴⁸⁹ & CMC³⁸⁶: | A322S |
| 48,964 bp | IVS10 + 6,000 G→T | |
| (exonic or non-coding | CMG-2³²²: Exon 11 G→T | |
| depending on isoform) | | |
| Intron 9 (40,154 bp) | IVS9 + 2T→C | splice |
| | | defect |

Described herein are also mutant CMG-2 coding nucleotide sequences, and mutant CMG-2 polypeptides that are encoded by such variant nucleic acids. Such mutant polypeptides comprise one or more amino acid residue substitutions, insertions or deletions. Such CMG-2 variant or variants can be characterized by a decreased CMG-2 function and/or activity; or by lower CMG-2 expression levels, as compared to controls.

In one embodiment, antibodies that specifically bind to variant CMG-2 polypeptides can be used in the methods of the invention to detect a variant CMG-2 polypeptide or CMG-2 gene expression product. In another embodiment, oligonucleotide sequences can be used, e.g., to detect a mutation in a CMG-2 gene, or to amplify a CMG-2 nucleic acid (for example, a specific locus on a CMG-2 gene) from a subject having or suspected of having a mutation that is associated with JHF and/or ISH, or a condition associated with a CMG-2 deficiency.

Methods are also provided, as part of the present invention, which use the nucleic acids, polypeptides and antibodies described herein to diagnose or treat JHF and/or ISH. For example, the invention provides methods to evaluate individuals for JHF and/or ISH by detecting a variant CMG-2 nucleic acid or CMG-2 polypeptide, such as one of the variants described herein, which is associated with JHF and/or ISH. The invention further provides methods to evaluate individuals for JHF and/or ISH by detecting a decreased CMG-2 activity, for example, by comparing CMG-2 activity to controls, or by detecting a variant CMG-2 polypeptide known to lead to a CMG-2 deficiency. In addition, the invention provides therapeutic methods for treating JHF, ISH, or a condition associated with CMG-2 deficiency, by administering a compound that provides or enhances CMG-2 activity or function. In one preferred embodiment, the compound is a wild-type CMG-2 nucleic acid or expression product, or a compound acting downstream in a pathway in which CMG-2 is a member.

Briefly, as described in the Examples, the CMG-2 gene, and JHF and ISH disease-causing mutations, were identified and characterized using the previously reported chromosome 4q21 JHF disease locus as a guide for candidate gene identification (Example 1). Two ISH family-specific truncating mutations, E220X and the 1 bp insertion P357insC which results in translation of an out-of-frame stop codon, were generated by site-directed mutagenesis and shown to delete the CMG-2 transmembrane and/or cytosolic domains, respectively. An ISH compound mutation, I189T, is predicted to create a novel and destabilizing internal cavity within the protein. The JHF family-specific homoallelic missense mutation G105D destabilizes a VWFA extracellular domain alpha helix while the other mutation, L329R, occurs within the protein's transmembrane domain (Example 2). Recombinant expression of the mutant CMG-2 sequences in HEK 293 cells showed that the mutant sequences were translated and expressed (Example 3). In addition, analysis of JHF and ISH patient-derived fibroblasts showed that the CMG-2 mutations abrogate normal cell interactions with the extracellular matrix protein (Example 4). Polymorphisms and mutations in non-coding regions were also identified in individuals not suffering from JHF or ISH, e.g., in family members to a JHF- or ISH-affected individual or individuals, or in subjects which had no known association to such an individual (Table 1B).

The discovery that CMG-2 mutations result in the allelic disorders JHF and ISH provides a non-invasive molecular diagnostic tool, defines these two diseases as being on either end of the same disease spectrum, and highlights novel information on the in vivo function of this integrin-like cell surface molecule and its role in key developmental and physiological processes. The dermal, gastrointestinal, and skeletal findings present in these syndromes could result from dysregulation in basement membrane architecture, possibly arising from compromised cell-matrix or cell-cell interactions. Histologic reports have identified cells embedded within a fibrillogranular material with cellularity inversely proportional to the lesion's age, and abnormal accumulation of extracellular deposits apparently originating from dermal blood vessels (Stucki et al., Am J Med Genet 2001;100:122-129). Without being bound to any specific theory, it is believed that CMG-2 plays an important role in basement membrane-matrix homeostasis and architecture during development and morphogenesis (Bell et al., J Cell Sci 2001; 114:2755-2773).

The following sequences are provided in the attached Sequence Listing, and can be used in accordance with the invention:

TABLE 2
Amino Acid and Nucleotide Sequences

| | |
|---|---|
| SEQ ID NO: 1 | CMG-2 genomic DNA sequence |
| SEQ ID NO: 2 | CMG-2-488 cDNA |
| SEQ ID NO: 3 | CMG-2-488 amino acid sequence |
| SEQ ID NO: 4 | CMG-2-386 cDNA |
| SEQ ID NO: 5 | CMG-2-386 amino acid sequence |
| SEQ ID NO: 6 | CMG-2-489 amino acid sequence |
| SEQ ID NO: 7 | CMG-2-322 amino acid sequence |
| SEQ ID NO: 8 | Amino acid sequence of CMG-2 |
| | P(357)insC variant |
| SEQ ID NOS: 9-25 | Exons 1-17, respectively |
| SEQ ID NOS: 26-51 | Primer pairs for exons 1-17 |
| SEQ ID NO: 52-67 | Microsatellite marker primers |
| SEQ ID NO: 68 | chain A of the Alpha-X Beta2 |
| | Integrin I Domain |

### DEFINITIONS

As used herein, the term “juvenile hyaline fibromatosis” (JHF) encompasses all forms of the disorder as described under the accession No. MIM 228600 in the Online Mendelian Inheritance in Man (OMIM) at World-Wide Web Address ncbi.nlm.nih.gov/Omim (as accessed in Aug. 26, 2003), and in the references cited therein. The references are as follows: Aldred et al., Oral Surg. Oral Med. Oral Path. 1987; 63: 71-77; Bedford et al., J. Pediat. 1991; 119: 404-410; Breier et al., Arch. Dis. Child. 1997; 77: 436-440; Dowling et al., Am. J. Hum. Genet. 2003; 73: 957-966; Drescher et al. Pediat. Surg. 1967; 2: 427-430; Enjoji et al., Acta Med. Univ. Kagoshima Suppl. 10: 1968; 145-151; Fayed et al., Am. J. Med. Genet. 1987; 26: 123-131; Gorlin et al., New York: Oxford Univ. Press (pub.) (3rd ed.) 1990. Pp. 849-850; Hanks et al., Am. J. Hum. Genet. 2003; 73: 791-800; Ishikawa et al., Arch. Klin. Exp. Derm. 1964; 218: 30-51; Keser et al., Clin. Rheum. 1999; 18: 248-252; Kitano et al., Arch. Derm. 1976; 112: 86-88; Kitano et al., Arch. Derm. 1972; 106: 877-883; Landing et al., Pediat. Path. 1986; 6: 55-79; Puretic et al., Brit. J. Derm. 1962; 74: 8-19; Rahman et al., Am. J. Hum. Genet. 2002; 71: 975-980; Roggli et al., Cancer 1980; 45: 954-960; Suschke et al., Dtsch. Med. Wschr. 1971; 96: 1941-1943; and Woyke et al., Cancer 1970; 26: 1157-1168.

JHF is a rare recessively inherited deforming disorder of head, neck, and generalized cutaneous nodules or tumors in children with normal mentality; the lesions consist of fibroblasts separated by an eosinophilic hyaline stroma composed mostly of glycosaminoglycans. Osteolytic lesions, osteoporosis, osteopenia, and arthritis are also associated with JHF.

The term “infantile systemic hyalinosis” (ISH) encompasses all forms of the disorder as described under the accession No. MIM 236490 OMIM database described above (as accessed on Aug. 26, 2003), and in the references cited therein. These references are as follows (excluding those which overlap with the one for JHF above): Nezelof et al., Arch. Franc. Pediat. 1978; 35: 1063-1074; and Stucki et al., Am. J. Med. Genet. 2001; 100: 122-129).

ISH is usually present at birth and is diagnosed in the first few weeks of life, and is characterized by deposits of hyaline material in skin, gastrointestinal tract, adrenals, urinary bladder, ovaries, skeletal muscles, thymus, parathyroids, and other loci. Clinical features include thickness and focal nodularity of skin, relatively short limbs and neck, gum hypertrophy, hypotonia and reduced movement, joint contractures, osteoporosis, arthritis, growth failure, diarrhea, and recurrent infections. ISH is more severe than JHF and is terminal.

The subject to whom the diagnostic or therapeutic applications of the invention are directed may be any human or animal, more particularly a mammal, preferably a primate or a rodent, but including, without limitation, monkeys, dogs, cats, horses, cows, pigs, sheep, goats, rabbits, guinea pigs, hamsters, mice and rats, including laboratory animals and genetically modified animals. The subject may be of any age, e.g., an adult, a child, an infant. Prenatal diagnostics and therapeutics interventions are also encompassed.

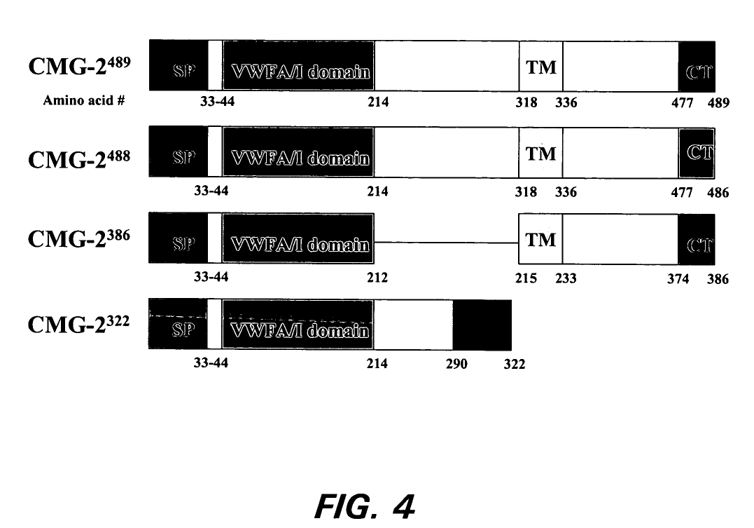

As used herein the term “CMG-2 protein” or “CMG-2 polypeptide” refers to gene products of the capillary morphogenesis gene-2 and homologs thereof, including isoforms and orthologs. This term includes CMG-2 protein isolated from a biological sample, synthetically produced, or recombinantly produced. CMG-2 encompasses CMG-2 protein of human origin, i.e., the CMG-2 protein having the sequence of SEQ ID NO:3, and CMG-2 isoforms having the sequences of SEQ ID NOS:5-7. This term further includes CMG-2 amino acid sequences described in U.S. patent application Publication 2002/0064831, published May 30, 2002, which is hereby incorporated by reference in its entirety. The differences in these isoforms are described in FIG. 4. For example, CMG-2⁴⁸⁸ (SEQ ID NO:2) is identical to CMG-2⁴⁸⁹ (SEQ ID NO:6) except that the last 12 amino acids of the cytoplasmic tail diverge in the two isoforms; CMG-2³⁸⁶ (SEQ ID NO:5) is identical to CMG-2⁴⁸⁹ except that it is missing amino acids 213-315; and CMG-2322 (SEQ ID NO:7) lacks, e.g., the cytoplasmic domains.

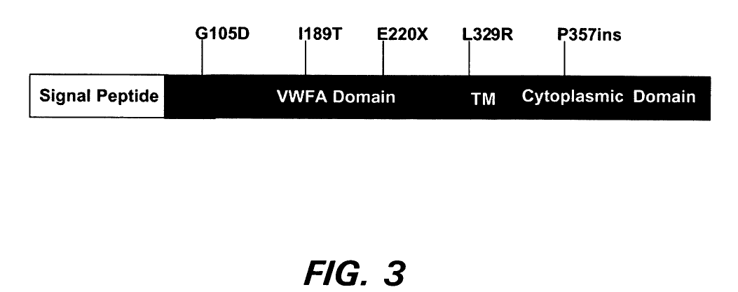

“CMG-2” also encompasses function-conservative variants and homologous proteins thereof, and proteins originating from different species. The term “CMG-2” refers to a peptide or protein sequence, whereas italicized “CMG-2” refers to a nucleotide sequence (genomic, cDNA, etc.). In a particular embodiment, a CMG-2 protein or polypeptide can be identified by comprising an amino acid sequence similar or identical to that of the signal peptide and VWFA domains (see FIGS. 3 and 4).

As used herein the term “CMG-2 nucleic acid” refers to a polynucleotide that encodes an CMG-2 polypeptide as described above, and homologs, including sequence-conservative variants, allelic variants and orthologs. One isoform is depicted in the GenBank database under the Accession No. AK091721. The CMG-2 cDNA sequence (from human fibroblasts) is depicted in SEQ ID NO:2, and the cDNA encoding the CMG-2³⁸⁶ isoform is depicted in SEQ ID NO:4. The genomic sequence of CMG-2 (SEQ ID NO:1) is organized into 17 exons, depicted in SEQ ID NOS: 9-25, exons 1-17, respectively. As used herein, this term also refers to nucleic acid CMG-2 primers or probes, i.e., nucleic acids comprising about 10-25 nucleotides of the sequence encoding a CMG-2 polypeptide.

A “CMG-2 gene” is used herein to refer to a portion of a DNA molecule that includes an CMG-2-polypeptide coding sequence operatively associated with expression control sequences. Thus, a gene includes both transcribed and untranscribed regions. The transcribed region may include introns, which are spliced out of the mRNA, and 5′- and 3′-untranslated (UTR) sequences along with protein coding sequences. In some embodiments, the gene can be a genomic or partial genomic sequence, in that it contains one or more introns. In other embodiments, the term gene may refer to a cDNA molecule (i.e., the coding sequence lacking introns).

“CMG-2 variant” nucleic acids are CMG-2 genomic DNA, cDNA, or mRNA comprising at least one mutation or polymorphism, such as a nucleotide substitution, deletion, or insertion. The nucleotide substitution may be in a coding or non-coding region. Preferred CMG-2 variants are those resulting in a CMG-2 deficiency as compared to a control. Any known wild-type or consensus CMG-2 sequence can be used as the reference sequence when identifying a mutation or polymorphism. For example, in one embodiment, CMG-2 mutations or polymorphisms are identified in reference to the CMG-2 genomic DNA sequence described herein as SEQ ID NO:1. Alternatively, any cDNA sequence encoding a CMG-2 isoform, including CMG-2-488 (SEQ ID NO:2), CMG-2-386 (SEQ ID NO:4), CMG-2-489, or CMG-2-322, can be used as reference.

“CMG-2 variant” polypeptides are CMG-2 proteins or polypeptides comprising at least one mutation or polymorphism. The CMG-2 variants can be function-conservative variants, including variants having an abrogated or reduced CMG-2 activity, such as a reduced ability to bind laminin, or a variant resulting in a reduced capability of a dermal fibroblast to bind to laminin or to convert MMP-2 into its active form. This may be assessed either by direct sequencing or detection of the mutation or measurement of CMG-2 activity (see, Example 4) and comparison to a reference sequence. Any known wild-type or consensus CMG-2 sequence can be used as the reference sequence when identifying a mutation or polymorphism. In one embodiment, CMG-2 mutations or polymorphisms are identified in reference to the CMG-2 amino acid sequence described herein as SEQ ID NO:3. In alternative embodiments, CMG-2 mutations or polymorphisms are identified in reference to the CMG-2 isoforms having the amino acid sequences described herein as SEQ ID NOS:5-7. Preferred mutations and polymorphisms are amino acid substitutions, deletions, and/or insertions, in particular those described in Tables 1A and 1B.

As used herein, the term “CMG-2 deficiency” refers to both deficient quantities of CMG-2 gene or CMG-2 protein expression, and reduced or abrogated GMG-2 protein activity (e.g., due to a truncation mutation leading to the loss of the transmembrane and cytosolic domains, or to inactivating mutation in a binding, transmembrane, or activation domain). Thus, a reduction in CMG-2 activity can result from the presence of less protein, or the presence of a normal amount of protein having lower activity as a result of a mutation or because of deregulation of its activity. Such CMG-2 deficiencies result in decreased CMG-2 function, and, in some embodiments, JHF and ISH pathology. In other embodiments, CMG-2 a CMG-2 deficiency can lead to a condition associated with a JHF- and/or ISH-associated pathology, such as, e.g., osteoporosis, osteolytic lesions, osteopenia, arthritis. Preferably, although not necessarily, “CMG-2 deficiency” is characterized by an CMG-2 expression or activity level of no more than 95%, preferably no more than 90%, more preferably no more than 50%, and even more preferably no more than 10% of the CMG-2 expression or activity level of a control.

The reduced or abrogated activity of CMG-2 in a test subject or a biological sample refers to a lower CMG-2 activity in the test subject or biological sample in comparison with a control, e.g., a healthy subject or a standard sample. A lower expression level of wild-type or variant CMG-2, resulting from, for example, a mutation or polymorphism in a non-coding region of a CMG-2 gene or a mutation in a coding or non-coding gene involved in CMG-2 transcription or translation, and can be determined by, e.g., comparing CMG-2 mRNA or level of CMG-2 protein in a test subject as compared to a control.

In a specific embodiment, the term “about” or “approximately” means within an acceptable error for the type of measurement used to obtain a value, e.g. within 20%, preferably within 10%, and more preferably within 5% of a given value or range. Alternatively, particularly with respect to biological systems or processes, the term means within an order of magnitude, and preferably a factor of two, of a value.

As used herein, the term “isolated” means that the referenced material is free of components present in the natural environment in which the material is normally found. In particular, isolated biological material is free of cellular components. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. A purified tumor cell is preferably substantially free of other normal cells. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

### Molecular Biology Terms

In accordance with the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

The terms “polypeptide” and “protein” may be used herein interchangeably to refer to the expression product (or corresponding synthetic product) of a message RNA (mRNA) encoded by a gene.

A “gene” is used herein to refer to a portion of a DNA molecule that includes a polypeptide-coding sequence operatively associated with expression control sequences. Thus, a gene includes both transcribed and untranscribed regions. The transcribed region may include introns, which are spliced out of the mRNA, and 5′- and 3′-untranslated (UTR) sequences along with protein coding sequences. In one embodiment, the gene can be a genomic or partial genomic sequence, in that it contains one or more introns. In another embodiment, the term gene may refer to a cDNA molecule (i.e., the coding sequence lacking introns). In yet another embodiment, the term gene may refer to expression control sequences, such as the promoter or the enhancer sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A “vector” is a recombinant nucleic acid construct, such as plasmid, phage genome, virus genome, cosmid, or artificial chromosome, to which another DNA segment may be attached. In a specific embodiment, the vector may bring about the replication of the attached segment, e.g., in the case of a cloning vector. A “replicon” is any genetic element (e.g., plasmid, chromosome, virus) that functions as an autonomous unit of DNA replication in vivo, i.e., it is capable of replication under its own control. The term “vector” includes both viral and nonviral means for introducing the nucleic acid into a cell in vitro, ex vivo or in vivo. Non-viral vectors include plasmids, liposomes, electrically charged lipids (cytofectins), DNA-protein complexes, and biopolymers. Viral vectors include retrovirus, adeno-associated virus, pox, baculovirus, vaccinia, herpes simplex, Epstein-Barr and adenovirus vectors. In addition to a nucleic acid according to the invention, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA is expressed and effects a function or phenotype on the cell in which it is expressed.

The term “heterologous” refers to a combination of elements not naturally occurring. For example, heterologous DNA refers to DNA not naturally located in the cell, or in a chromosomal site of the cell. A heterologous expression regulatory element is such an element operatively associated with a different gene than the one it is operatively associated with in nature, such as a CMV promoter operatively associated with a CMG-2 coding region. In the context of the present invention, a CMG-2 gene is heterologous to vector DNA in which it is inserted for cloning or expression.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 1987;50:667). Such proteins (and their encoding genes) have sequence homology, as reflected by their sequence similarity, whether in terms of percent similarity or the presence of specific residues or motifs at conserved positions.

The term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90% or at least 95%, 96%, 97%, 98% or 99%) of the nucleotides match over the defined length of the DNA sequences, as determined by sequence comparison algorithms, such as BLAST, FASTA, DNA Strider, etc. An example of such a sequence is an allelic or species variant of the CMG-2 gene. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90%, 91%, 92%, 93%, 94%, 95%, 96%, 97%, 98%, or 99% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc.).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., infra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. SSC is a 0.15M NaC1, 0.015M Na-citrate. Hybridization requires that the two nucleic acids contain complementary sequences. Depending on the stringency of the hybridization, however, mismatches between bases are possible. The appropriate stringency for hybridizing nucleic acids depends on the length of the nucleic acids and the degree of complementarity, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., infra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Electronic database information for use in accordance with the present invention can be found, for example, on the World-Wide-Web (www.) sites of the following entities: Celera, for identification of candidate genes (celera.com); Decode, for the human genetic map (decodegenetics.com); Ensembl, for the identification of candidate genes (ensembl.org); Genome Database, for microsatellite markers (gdbwww. followed by gdb.org); and Online Mendelian Inheritance in Man, for definitions and descriptions of various inherited disorders (OMIM; ncbi.nlm.nih.gov/Omim), including JHF (MIM 228600); ISH (MIM 236490); Epidermolysis bullosa with pyloric atresia (MIM 226730); and Multiple epiphyseal dysplasia (MIM 607068).

“Amplification” of DNA as used herein encompasses the use of polymerase chain reaction (PCR) to increase the concentration of a particular DNA sequence within a mixture of DNA sequences. For a description of PCR see Saiki et al., Science 1988, 239:487.

“Sequencing” of a nucleic acid includes chemical or enzymatic sequencing. “Chemical sequencing” of DNA denotes methods such as that of Maxam and Gilbert (Maxam-Gilbert sequencing, Maxam and Gilbert, Proc Natl Acad Sci USA 1977;74:560), in which DNA is randomly cleaved using individual base-specific reactions. “Enzymatic sequencing” of DNA denotes methods such as that of Sanger (Sanger et al., Proc Natl Acad Sci USA 1977;74:5463), in which a single-stranded DNA is copied and randomly terminated using DNA polymerase, including variations thereof, which are well-known in the art. Preferably, oligonucleotide sequencing is conducted using automatic, computerized equipment in a high-throughput setting, for example, microarray technology, as described herein. Such high-throughput equipment are commercially available, and techniques well known in the art.

The terms “mutant” and “mutation” mean any detectable change in genetic material, e.g., DNA, or any process, mechanism, or result of such a change. As used herein, a mutation is known to be associated with a disease phenotype, such as, e.g., JHF and/or ISH. When compared to a control material, such change may be referred to as an “abnormality”. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant linked with a disease.

The term “polymorphism” refers, generally, to the coexistence of more than one form of a gene (e.g., more than one allele) within a population of individuals. As used herein, a polymorphism is generally not conclusively linked to a disease, but may or may not be associated with an increased risk for a disease or condition. The different alleles may differ at one or more positions of their nucleic acid sequences, which are referred to herein as “polymorphic locuses”. When used herein to describe polypeptides that are encoded by different alleles of a gene, the term “polymorphic locus” also refers to the positions in an amino acid sequence that differ among variant polypeptides encoded by different alleles. Polymorphisms include “single nucleotide polymorphisms” (SNPs), referring to a polymorphic site occupied by a single nucleotide, which is the site of variation between allelic sequences. Typically, the polymorphic site of an SNP is flanked by highly conserved sequences (e.g., sequences that vary in less than 1/100 and, more preferably, in less than 1/1000 individuals in a population). The polymorphic locus of an SNP may be a single base deletion, a single base insertion, or a single base substitution.

As used herein, “sequence-specific oligonucleotides” refers to related sets of oligonucleotides that can be used to detect variations or mutations in the CMG-2 gene.

A “probe” refers to a nucleic acid or oligonucleotide that forms a hybrid structure with a sequence in a target region due to complementarity of at least one sequence in the probe with a sequence in the target protein.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of CMG-2, or to detect the presence of nucleic acids encoding CMG-2. In a further embodiment, an oligonucleotide of the invention can form a triple helix with a CMG-2 DNA molecule. In still another embodiment, a library of oligonucleotides arranged on a solid support, such as a silicon wafer or chip, can be used to detect various mutations of interest. Generally, oligonucleotides are prepared synthetically, preferably on a nucleic acid synthesizer. Accordingly, oligonucleotides can be prepared with non-naturally occurring phosphoester analog bonds, such as thioester bonds, etc.

Specific non-limiting examples of synthetic oligonucleotides envisioned for this invention include oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH)₃—O—CH₂, CH₂—O—N(CH)₃—CH₂, CH₂—N(CH)₃—N(CH)₃—CH₂ and O—N(CH)₃—CH₂—CH₂ backbones (where the phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 1991;254:1497). Other synthetic oligonucleotides may contain substituted sugar moieties comprising one of the following at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—; S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.

The term “linkage” refers to the tendency of genes, alleles, loci or genetic markers to be inherited together as a result of their location on the same chromosome. Linkage may be measured, e.g., by the percent recombination between two genes, alleles, loci or genetic markers.

### Expression of CMG-2 Polypeptides

A nucleotide sequence coding for CMG-2, for an antigenic fragment, derivative or analog of CMG-2, of for a functionally active derivative of CMG-2 (including a chimeric protein) may be inserted into an appropriate expression vector, i.e., a vector which contains the necessary elements for the transcription and translation of the inserted protein-coding sequence. Such cells, comprising variant CMG-2 genes or expressing variant CMG-2 polypeptides, can be useful for a variety of purposes, including antibody production and drug discovery, such as in, e.g., screening methods to identify substances that modify CMG-2 translation or transcription, or that otherwise alleviate a condition caused by the CMG-2 deficiency in the cell.

Thus, a nucleic acid encoding a CMG-2 polypeptide of the invention can be operationally associated with a promoter in an expression vector of the invention. Both cDNA and genomic sequences can be cloned and expressed under control of such regulatory sequences. Such vectors can be used to express functional or functionally inactivated CMG-2 polypeptides. In particular, the CMG-2 nucleic acids which may be cloned and expressed according to these methods include, not only wild-type CMG-2 nucleic acids, but also variant or variant CMG-2 nucleic acids. These include, for example, a CMG-2 nucleic acid having one or more of the mutations set forth in Tables 1A and 1B. In addition, nucleic acids that encode a variant CMG-2 polypeptide, for example a variant CMG-2 polypeptide comprising one or more of the amino acid substitutions listed in Tables 1A and 1B may be cloned and expressed according to the methods described here.

The necessary transcriptional and translational signals can be provided on a recombinant expression vector. Potential host-vector systems include but are not limited to mammalian cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of a CMG-2 protein, including CMG-2 variants, may be controlled by any promoter/enhancer element known in the art, but these regulatory elements must be functional in the host selected for expression. Promoters which may be used to control CMG-2 gene expression include, but are not limited to, cytomegalovirus (CMV) promoter (U.S. Pat. Nos. 5,385,839 and 5,168,062), the SV40 early promoter region (Benoist and Chambon, Nature 1981;290:304-310), the promoter contained in the 3′ long terminal repeat of Rous sarcoma virus (Yamamoto et al., Cell 1980;22:787-797), the herpes thymidine kinase promoter (Wagner et al., Proc Natl Acad Sci U.S.A. 1981;78:1441-1445), the regulatory sequences of the metallothionein gene (Brinster et al., Nature 1982;296:39-42); prokaryotic expression vectors such as the beta-lactamase promoter (Villa-Komaroff et al., Proc Natl Acad Sci U.S.A. 1978;75:3727-3731), or the tac promoter (DeBoer et al., Proc Natl Acad Sci U.S.A. 1983;80:21-25); see also “Useful proteins from recombinant bacteria” in Scientific American 1980;242:74-94. Still other useful promoter elements which may be used include promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 1985;315:338-340; Kollias et al., Cell 1986;46:89-94), hematopoietic stem cell differentiation factor promoters, erythropoietin receptor promoter (Maouche et al., Blood 1991;15:2557), etc.

Soluble forms of the protein can be obtained by collecting culture fluid, or solubilizing-inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2 dimensional gel electrophoresis, chromatography (e.g., ion exchange, affinity, immunoaffinity, and sizing column chromatography), centrifugation, differential solubility, immunoprecipitation, or by any other standard technique for the purification of proteins.

A wide variety of host/expression vector combinations may be employed in expressing the DNA sequences of this invention. Useful expression vectors, for example, may consist of segments of chromosomal, non chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 1988;67:31-40), pCR2.1 and pcDNA 3.1+ (Invitrogen, Carlsbad, Calif.), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

One type of suitable vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or variant CMG-2 protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures (see below), as well as in vitro expression, are DNA-based vectors and retroviral vectors. Methods for constructing and using viral vectors are known in the art (see, e.g., Miller and Rosman, BioTechniques 1992;7:980-990). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), or can be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), baculovirus, and the like. RNA viral vectors include, for example, retroviruses, lentiviruses, and alphaviruses (e.g., Sindbis virus and Venezuelan Equine Encephalitis virus), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Mol Cell Neurosci 1991;2:320-330), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al.(J Clin Invest 1992;90:626-630; see also La Salle et al., Science 1993;259:988-990); and a defective adeno-associated virus vector (Samulski et al., J Virol 1987;61:3096-3101; Samulski et al., J Virol 1989;63:3822-3828; Lebkowski et al., Mol Cell Biol 1988;8:3988-3996) and Lieber et al., J Virol 1999;73:9314-24.

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), Transgene (Strasbourg, France; adenoviral, vaccinia, retroviral, and lentiviral vectors) and Invitrogen (Carlbad, Calif.).

In another embodiment, the vector can be introduced into a cell, in vitro or in vivo, by lipofection, as naked DNA, or with other transfection facilitating agents (peptides, polymers, etc.). Synthetic cationic lipids can be used to prepare liposomes for in vivo transfection of a gene encoding a marker (Felgner et al., Proc Natl Acad Sci U.S.A. 1987;84:7413-7417; Felgner and Ringold, Science 1989;337:387-388; Mackey et al., Proc Natl Acad Sci U.S.A. 1988;85:8027-8031; Ulmer et al., Science 1993;259:1745-1748). Useful lipid compounds and compositions for transfer of nucleic acids are described in International Patent Publications WO 95/18863 and WO 96/17823, and in U.S. Pat. No. 5,459,127. Lipids may be chemically coupled to other molecules for the purpose of targeting (see, Mackey et al., Proc Natl Acad Sci U.S.A. 1988;85:8027-8031). Targeted peptides, and proteins such as antibodies, or non-peptide molecules could be coupled to liposomes chemically. Other molecules are also useful for facilitating transfection of a nucleic acid in vivo, such as a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce the vector in vivo as a naked DNA plasmid. Naked DNA vectors for gene therapy can be introduced into the desired host cells by methods known in the art; e.g., electroporation, microinjection, cell fusion, DEAE dextran, calcium phosphate precipitation, use of a gene gun, or use of a DNA vector transporter (see, e.g., Wu et al., J Biol Chem 1992, 267:963-967; Wu and Wu, J Biol Chem 1988;263:14621-14624; Hartmut et al., Canadian Patent Application No. 2,012,311, filed Mar. 15, 1990; Williams et al., Proc Natl Acad Sci U.S.A. 1991;88:2726-2730). Receptor-mediated DNA delivery approaches can also be used (Curiel et al., Hum Gene Ther 1992;3:147-154; Wu and Wu, J Biol Chem 1987;262:4429-4432). U.S. Pat. Nos. 5,580,859 and 5,589,466 disclose delivery of exogenous DNA sequences, free of transfection facilitating agents, in a mammal. A relatively low voltage, high efficiency in vivo DNA transfer technique, termed electrotransfer, has been described (Mir et al., C.P. Acad Sci 1998;321:893; WO 99/01157; WO 99/01158; WO 99/01175).

For in vivo administration, an appropriate immunosuppressive treatment can be employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors (see, e.g., Wilson, Nat Med 1995;1:887-889). It may also be advantageous to employ a viral vector that is engineered to express a minimal number of antigens.

### Diagnostic Methods

According to the present invention, mutations or polymorphisms in the CMG-2 gene leading to a CMG-2 deficiency are linked to certain syndromes and conditions. For example, polymorphisms in the CMG-2 gene can be detected and linked with an increased risk for conditions such as osteolytic lesions, osteoporosis, osteopenia, arthritis, and other conditions observed in JHF and/or ISH. In addition, mutated forms of CMG-2 can be detected to diagnose JFH and/or IHS. Diagnostic methods may comprise, for example, detecting a mutation or polymorphism in a CMG-2 gene, wherein the mutation or polymorphism results in decreased CMG-2 expression or activity. The mutation or polymorphism may especially affect a coding region of the gene. The mutation or polymorphism may be a missense mutation, preferably a missense mutation resulting in nucleic acid substitution, or a deletion, insertion, or a combination of two or more mutations. Preferably, the mutation or polymorphism results in one or more of the amino acid substitutions or truncations/deletions/insertions set forth in Tables 1A and 1B. Most preferably, the nucleotide substitutions, insertions, or deletions are selected from the ones described in Tables 1A and 1B.

The diagnostic methods of the invention also encompass detecting a CMG-2 variant, in particular a variant having decreased CMG-2 activity. The variant may have a mutation or polymorphism, such as an amino acid or polypeptide substitution or truncation. Preferred amino acid substitutions and deletions for diagnostic use are set forth in Table 1A.

In a further embodiment, the diagnosis of JFH or IHS in a subject comprises assessing the level of expression or activity of a wild-type or consensus CMG-2 protein in the test subject and comparing it to the level of expression or activity in a control subject, wherein an decreased expression and/or activity of the CMG-2 protein in the test subject compared to the control subject is indicative of JFH or IHS, or a condition associated with one or more of these disorders.

The level of expression of CMG-2 may be assessed by determining the amount of mRNA that encodes the CMG-2 protein in a biological sample, or by determining the concentration of CMG-2 protein in a biological sample. The level of CMG-2 protein or activity may also be assessed by determining the laminin-binding capability of patient fibroblasts, or the capability of the patient fibroblasts to convert the inactive pro-form of MMP-2 to active MMP-2. In an alternative embodiment, a secreted form of a CMG-2 protein, such as e.g., the CMG-2-322 isoform, can be detected in a body fluid.

The invention also provides kits for performing these diagnostic methods. A particular subject of the invention is a kit for diagnosing JHF and/or ISH, comprising an oligonucleotide that specifically hybridizes to a site harboring a mutation of the CMG-2 gene, or an adjacent site, wherein the mutation results in decreased basal activity of the CMG-2 protein. The site of mutation may particularly comprise a nucleotide selected from the group consisting of the nucleotides corresponding to G37632, G17627, T65089, C88794, T19288, C88790, and 48964 of SEQ ID NO:3, or any nucleotide recited in Tables 1A and 1B, as described below. A further subject of the invention is a kit for diagnosing JHF and/or ISH, or a condition observed in individuals suffering from these disorders, such as, e.g., osteoporosis or arthritis, comprising an antibody that specifically recognizes a mutated form of CMG-2 protein that results in increased basal activity of the protein.

As used herein, the term “diagnosis” refers to the identification of a disease or condition at any stage of its development, and also includes the determination of a predisposition of a subject to develop the disease or condition. Importantly, the invention permits genetic counseling of prospective parents and in utero genetic testing for JHF and ISH syndromes. Families with one affected parent or with advanced paternal age are of particular concern. The diagnostic method of the invention also allows confirmation of a questionable JHF or ISH diagnosis based on phenotype (appearance and symptomology). The diagnostic method of the invention may also be envisioned in the case of fetal abnormalities whose cause may not be obvious, or in the case of fetal loss, to evaluate viability of future pregnancies. Further, the risk or propensity for a non-JHF and non-ISH individual to develop a condition associated with JHF or ISH, such as osteoporosis or arthritis, can be estimated based on the detection of a CMG-2 variant or CMG-2 deficiency.

The term “biological sample” refers to any cell source from which CMG-2 DNA or CMG-2 protein may be obtained. Non-limiting examples of cell sources available in clinical practice include without limitation blood cells, dermal cells (e.g., fibroblasts), buccal cells, cervicovaginal cells, epithelial cells from urine, fetal cells, or any cells present in tissue obtained by biopsy. Cells may also be obtained from body fluids, including without limitation blood, plasma, serum, lymph, milk, cerebrospinal fluid, saliva, sweat, urine, feces, and tissue exudates (e.g., pus) at a site of infection or inflammation. For prenatal testing, genetic material can be obtained from fetal cells, e.g., from amniotic fluid (through amniocentesis), chronic villi, blood, or any tissue of a pregnant woman. DNA is extracted using any of the numerous methods that are standard in the art. It will be understood that the particular method used to extract DNA will depend on the nature of the source. Generally, the minimum amount of DNA to be extracted for use in the present invention is about 25 pg (corresponding to about 5 cell equivalents of a genome size of 4×10⁹ base pairs). The CMG-2 gene has been found to be fairly ubiquitously expressed in the body, except for in brain and thymus.

Various methods for detecting variant forms of CMG-2 are described herein. The present invention especially contemplates detecting abnormalities, i.e., mutations or polymorphisms in the CMG-2 gene that result in an decreased basal activity of the CMG-2 protein, render the protein in a inactive conformation, results in a truncated form of CMG-2, or decreases the level of expressed CMG-2 protein. Mutations and polymorphisms may include an insertion in the gene, a truncation of or deletion in the gene, a nonsense mutation, a frameshift mutation, a splice-site mutation, and a missense mutation. Such variations can occur in the coding region of the CMG-2 gene, more particularly in any of the functional domains, as well as in the untranslated regions, more particularly in the promoter or enhancer regions. Preferred mutations or polymorphisms are those in any of exons 4, 7, 8, 11, 12 or 13, and those in introns 4, 6, 7, 9, 13, 16, and the 3′ UTR region. Even more preferred are mutations resulting in amino acid substitutions or truncations. Specific mutations are listed in Tables 1A and 1B.

### Nucleic Acid Based Assays

According to the invention, variant forms of CMG-2 nucleic acids, i.e. in the CMG-2 DNA or in its transcripts, as well as a deregulated expression, e.g. decreased expression, of CMG-2 can be detected by a variety of suitable methods. Standard methods for analyzing the nucleic acid contained in a biological sample and for diagnosing a genetic disorder can be employed, and many strategies for genotypic analysis are known to those of skilled in the art. In a preferred embodiment, the determination of mutations in the CMG-2 gene encompasses the use of nucleic acid sequences such as specific oligonucleotides, to detect mutations in CMG-2 genomic DNA or mRNA in a biological sample. Such oligonucleotides may specifically hybridize to a site of mutation or polymorphism, or to a region adjacent to this site of mutation or polymorphism present in a CMG-2 nucleic acid. One may also employ primers that permit amplification of all or part of CMG-2. Alternatively, or in combination with such techniques, oligonucleotide sequencing described herein or known to the skilled artisan can be applied to detect the CMG-2 mutations or polymorphisms.

One skilled in the art may use hybridization probes in solution and in embodiments employing solid-phase procedures. In embodiments involving solid-phase procedures, the test nucleic acid is adsorbed or otherwise affixed to a selected matrix or surface. The fixed, single-stranded nucleic acid is then subjected to specific hybridization with selected probes.

In another embodiment, one skilled in the art may use oligonucleotide primers in an amplification technique, such as PCR or reverse-PCR (“reverse polymerase chain reaction”), to specifically amplify the target DNA or mRNA, respectively, that is potentially present in the biological sample. Useful oligonucleotides include primers that permit amplification of CMG-2 exons or introns. The following are exemplary primers for amplifying CMG-2 exons (where the preceding number or numbers refers to the exon(s) amplified, “F” indicates forward primer; and “R” indicates reverse primer):

SEQ ID
NO:

| | |
|---|---|
| Exon 1F: AGA GTG CGT GCC GGG TGA | 26 |
| Exon 1R: GAA AGA AGA CAG CAA CAG GGC ACC | 27 |
| Exon 2F: GAC GGA GTC TTG CTC TGG GAC | 28 |
| Exon 2R: GTG CAA TAC GAC CTT GAG GCA | 29 |
| Exon 3F: CTG GAC CAT TCA GTG AGA CC | 30 |
| Exon 3R: GCC TGA ATC ACC ACT TGG AA | 31 |
| Exon 4,5,6F: AGC TTA GTT ACA ATA CTG CCA TG | 32 |
| Exon 4,5,6R: CCA GTG TCA CAA TGT CAT CAG | 33 |
| Exon 7F: GCC AAC TTA AAG GTA CTC TGA CTG | 34 |
| Exon 7R: TCT AGA TAA TGA CCA CCT GCA CTG | 35 |
| Exon 8F: GAA GTA TGG AGA AGA CCT CAA GG | 36 |
| Exon 8R: GCC TGT CAC ACA ATA TGC TC | 37 |
| Exon 9F: GGA AAG CCA GCA CAG TTG G | 38 |
| Exon 9R: TGC TGA TGT GCT TTG CAG AG | 39 |
| Exon 10F: TGA ACT CTG ATT GAA GCA TGC | 40 |
| Exon 10R: GGC TTG CCC AAG GCT TAC | 41 |
| Exon 11F CAG GAG TTT GAG ACC CTT ACT C | 42 |
| Exon 11R CCA TAG ATT ATT TCT GGA TGG AAT TGC | 43 |
| Exon 12F GGA ATT TGA CCA TAA GCT GTG C | 44 |
| Exon 12R GAA ACT TTG CTG TTA TTA ACA TGG CA | 45 |
| Exon 13,14F GAC TTC TTT GGA GCT ACC ACA | 46 |
| Exon 13,14R GCC CTA GAA ATA CAT ACT CCA GA | 47 |
| Exon 15,16F CTC TGA GAT GTG AAC TAA AGG ACC | 48 |
| Exon 15,16R GGG CTG ATG CAA TGA TTG TGC | 49 |
| Exon 17F GAC TTC ATG TCT CAA GTT AAC ATG G | 50 |
| Exon 17R CAG AAG GCA GAG AAA ACA TTT CC | 51 |

The present invention is more particularly directed to a method of in vitro diagnosis of JHF and/or ISH, or a condition associated therewith, comprising the steps of: (a) contacting a biological sample containing DNA with specific oligonucleotides permitting the amplification of all or part of the CMG-2 gene, the DNA contained in the sample having being rendered accessible, where appropriate, to hybridization, and under conditions permitting a hybridization of the primers with the DNA contained in the biological sample; (b) amplifying said DNA; (c) detecting the amplification products; and (d) comparing the amplified products as obtained to the amplified products obtained with a normal control biological sample, and thereby detecting a possible abnormality in the CMG-2 gene.

The method of the invention can also be applied to the detection of an abnormality in the transcript of the CMG-2 gene, e.g. by amplifying the mRNAs contained in a biological sample, for example by RT-PCR. Thus another subject of the present invention is a method of in vitro diagnosis of JHF, ISH, or a condition associated therewith, as previously defined comprising the steps of: (a) producing cDNA from mRNA contained in a biological sample; (b) contacting said cDNA with specific oligonucleotides permitting the amplification of all or part of the transcript of the CMG-2 gene, under conditions permitting a hybridization of the primers with said cDNA; (c) amplifying said cDNA; (d) detecting the amplification products; and (e) comparing the amplified products as obtained to the amplified products obtained with a normal control biological sample, and thereby detecting a possible abnormality in the transcript of the CMG-2 gene.

For RNA analysis, the biological sample may be any cell source, as described above, such as a biopsy tissue, from which RNA is isolated using standard methods well known to those of ordinary skill in the art such as guanidium thiocyanate-phenol-chloroform extraction (Chomocyznski et al., Anal. Biochem. 1987;162:156). The isolated RNA is then subjected to coupled reverse transcription and amplification by polymerase chain reaction (RT-PCR), using specific oligonucleotide primers that are specific for a selected site. Conditions for primer annealing are chosen to ensure specific reverse transcription and amplification; thus, the appearance of an amplification product is diagnostic of the presence of a particular genetic variation. In another embodiment, RNA is reverse-transcribed and amplified, after which the amplified sequences are identified by, e.g., direct sequencing. In still another embodiment, cDNA obtained from the RNA can be cloned and sequenced to identify a mutation.

The CMG-2 nucleic acids of the invention can also be used as probes, e.g., in therapeutic and diagnostic assays. For instance, the present invention provides a probe comprising a substantially purified oligonucleotide, which oligonucleotide comprises a region having a nucleotide sequence that is capable of hybridizing specifically to a region of a CMG-2 gene which differs from that of a wild-type or consensus gene such as SEQ ID NO:1, e.g., a mutant or polymorphic region. Such probes can then be used to specifically detect which mutation or polymorphism of the CMG-2 gene is present in a sample taken from a subject. The mutant or polymorphic region can be located in the promoter, exon, intron, or UTR sequences of the CMG-2 gene.

For example, preferred probes of the invention include one or more of the nucleotide substitutions listed in Tables 1A and 1B, as well as the wild-type flanking regions (see, e.g., SEQ ID NO: 1). For each such probe; the complement of that probe is also included in the Table as a preferred probe of the invention. Particularly preferred probes of the invention have a number of nucleotides sufficient to allow specific hybridization to the target nucleotide sequence. Thus, probes of suitable lengths based on SEQ ID NO:1 and complementary to the variant sequences provided herein can be constructed and tested by the skilled artisan for appropriate level of specificity depending on the application intended. Where the target nucleotide sequence is present in a large fragment of DNA, such as a genomic DNA fragment of several tens or hundreds of kilobases, the size of the probe may have to be longer to provide sufficiently specific hybridization, as compared to a probe which is used to detect a target sequence which is present in a shorter fragment of DNA. For example, in some diagnostic methods, a portion of the CMG-2 gene may first be amplified and thus isolated from the rest of the chromosomal DNA and then hybridized to a probe. In such a situation, a shorter probe will likely provide sufficient specificity of hybridization. For example, a probe having a nucleotide sequence of about 10 nucleotides may be sufficient, although probes of about 15 nucleotides, even more preferably 20 nucleotides, are preferred.

In a preferred embodiment, the probe or primer further comprises a label attached thereto, which preferably is capable of being detected. The label can, for example, be selected from radioisotopes, fluorescent compounds, enzymes, and enzyme co-factors. In another preferred embodiment of the invention, the isolated nucleic acid, which is used, e.g., as a probe or a primer, is modified, such as to become more stable. Exemplary nucleic acid molecules which are modified include phosphoramidate, phosphothioate and methylphosphonate analogs of DNA (see also U.S. Pat. Nos. 5,176,996; 5,264,564; and 5,256,775).

In yet another embodiment, one may use HPLC or denaturing HPLC (DHPLC) techniques to analyze the CMG-2 nucleic acids. DHPLC was developed when observing that, when HPLC analyses are carried out at a partially denaturing temperature, i.e., a temperature sufficient to denature a heteroduplex at the site of base pair mismatch, homoduplexes can be separated from heteroduplexes having the same base pair length (Hayward-Lester et al., Genome Research 1995;5:494; Underhill, et al., Proc Natl Acad Sci USA 1996;93:193; Doris et al., DHPLC Workshop 1997, Stanford University). Thus, the use of DHPLC was applied to mutation detection (Underhill et al., Genome Research 1997;7:996; Liu et al., Nucleic Acid Res 1998;26:1396). DHPLC can separate heteroduplexes that differ by as little as one base pair. “Matched Ion Polynucleotide Chromatography” (MIPC), or Denaturing “Matched Ion Polynucleotide Chromatography” (DMIPC) as described in U.S. Pat. Nos. 6,287,822 or 6,024,878, are separation methods that can also be useful in connection with the present invention.

Alternatively, one can use the DGGE method (Denaturing Gradient Gel Electrophoresis), or the SSCP method (Single Strand Conformation Polymorphism) for detecting an abnormality in the CMG-2 gene. DGGE is a method for resolving two DNA fragments of identical length on the basis of sequence differences as small as a single base pair change, using electrophoresis through a gel containing varying concentrations of denaturant (Guldberg et al., Nucleic Acid Res. 1994;22:880). SSCP is a method for detecting sequence differences between two DNAs, comprising hybridization of the two species with subsequent mismatch detection by gel electrophoresis (Ravnik-Glavac et al., Hum Mol Genet 1994;3:801). “HOT cleavage”, a method for detecting sequence differences between two DNAs, comprising hybridization of the two species with subsequent mismatch detection by chemical cleavage (Cotton et al., Proc Natl Acad Sci USA 1988;85:4397), can also be used. Such methods are preferably followed by direct sequencing. Advantageously, the RT-PCR method may be used for detecting abnormalities in the CMG-2 transcript, as it allows to visualize the consequences of a splicing mutation such as exon skipping or aberrant splicing due to the activation of a cryptic site. Preferably this method is followed by direct sequencing as well.

More recently developed techniques using microarrays, preferably microarray techniques allowing for high-throughput screening, can also be advantageously implemented for detecting an abnormality in the CMG-2 gene or for assaying expression of the CMG-2 gene. Microarrays may be designed so that the same set of identical oligonucleotides is attached to at least two selected discrete regions of the array, so that one can easily compare a normal sample, contacted with one of said selected regions of the array, against a test sample, contacted with another of said selected regions. These arrays avoid the mixture of normal sample and test sample, using microfluidic conduits. Useful microarray techniques include those developed by Nanogen, Inc (San Diego, Calif.) and those developed by Affymetrix. However, all types of microarrays, also called “gene chips” or “DNA chips”, may be adapted for the identification of mutations. Such microarrays are well known in the art (see for example the following: U.S. Pat. Nos. 6,045,996; 6,040,138; 6,027,880; 6,020,135; 5,968,740; 5,959,098; 5,945,334; 5,885,837; 5,874,219; 5,861,242; 5,843,655; 5,837,832; 5,677,195 and 5,593,839).

The solid support on which oligonucleotides are attached may be made from glass, silicon, plastic (e.g., polypropylene, nylon), polyacrylamide, nitrocellulose, or other materials. One method for attaching the nucleic acids to a surface is by printing on glass plates, as is described generally by Schena et al., Science 1995;270:467-470. This method is especially useful for preparing microarrays of cDNA. See also DeRisi et al., Nature Genetics 1996;14:457-460; Shalon et al., Genome Res. 1996;6:639-645; and Schena et al., Proc. Natl. Acad. Sci. USA 1995;93:10539-11286. Another method of making microarrays is by use of an inkjet printing process to bind genes or oligonucleotides directly on a solid phase, as described, e.g., in U.S. Pat. No. 5,965,352.

Other methods for making microarrays, e.g., by masking (Maskos and Southern, Nuc. Acids Res. 1992;20:1679-1684), may also be used. In principal, any type of array, for example, dot blots on a nylon hybridization membrane (see Sambrook et al., Molecular Cloning A Laboratory Manual (2nd Ed.), Vol. 1-3, Cold Spring Harbor Laboratory, Cold Spring Harbor, N.Y., 1989) could be used, although, as will be recognized by those of skill in the art, very small arrays will be preferred because hybridization volumes will be smaller. For these assays nucleic acid hybridization and wash conditions are chosen so that the attached oligonucleotides “specifically bind” or “specifically hybridize” to at least a portion of the CMG-2 gene present in the tested sample, i.e., the probe hybridizes, duplexes or binds to the CMG-2 locus with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base [symbol] pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., Science 1996;274:610-614).

A variety of methods are available for detection and analysis of the hybridization events. Depending on the reporter group (fluorophore, enzyme, radioisotope, etc.) used to label the DNA probe, detection and analysis are carried out fluorimetrically, calorimetrically or by autoradiography. By observing and measuring emitted radiation, such as fluorescent radiation or a particle emission, information may be obtained about the hybridization events.

When fluorescently labeled probes are used, the fluorescence emissions at each site of transcript array can, preferably be detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., Genome Res. 1996;6:639-695).

### Protein Based Assays

As an alternative to analyzing CMG-2 nucleic acids, one can evaluate CMG-2 on the basis of mutations or polymorphisms in the protein, or dysregulated production, e.g. decreased production, of one or more isoforms of the CMG-2 protein. In one embodiment, the ability of patient fibroblasts to bind laminin or other ligands, and/or to process MMP-2 can be evaluated to determine decreased expression or activity of CMG-2. In another embodiment, patient fibroblast can be used for assessing the binding of a CMG-2 ligand from, e.g., serum or identified as described below, wherein a lack of binding or lack of cellular response to binding indicates a CMG-2 deficiency.

In preferred embodiments, CMG-2 is detected by immunoassay. For example, Western blotting permits detection of a specific variant, or the presence or absence of CMG-2. In particular, an immunoassay can detect a specific (wild-type or variant) amino acid sequence in a CMG-2 protein. Other immunoassay formats can also be used in place of Western blotting, as described below for the production of antibodies. One of these is ELISA assay. In ELISA assays, an antibody against wild-type or consensus CMG-2 or against an epitopic fragment of CMG-2 is immobilized onto a selected surface, for example, a surface capable of binding proteins such as the wells of a polystyrene microtiter plate. After washing to remove incompletely adsorbed polypeptides, a nonspecific protein such as a solution of bovine serum albumin (BSA) may be bound to the selected surface. This allows for blocking of nonspecific adsorption sites on the immobilizing surface and thus reduces the background caused by nonspecific bindings of antisera onto the surface. The immobilizing surface is then contacted with a sample, to be tested in a manner conductive to immune complex (antigen/antibody) formation. This may include diluting the sample with diluents, such as solutions of BSA, bovine gamma globulin (BGG) and/or phosphate buffered saline (PBS)/Tween. The sample is then allowed to incubate for from 2 to 4 hours, at temperatures between about 25° to 37° C. Following incubation, the sample-contacted surface is washed to remove non-immunocomplexed material. The washing procedure may include washing with a solution, such as PBS/Tween or borate buffer. Following formation of specific immunocomplexes between the test sample and the bound antibody, and subsequent washing, the occurrence, and an even amount of immunocomplex formation may be determined by subjecting the immunocomplex to a second antibody against CMG-2, that recognizes a different epitope on the protein. To provide detecting means, the second antibody may have an associated activity such as an enzymatic activity that will generate, for example, a color development upon incubating with an appropriate chromogenic substrate. Quantification may then be achieved by measuring the degree of color generation using, for example, a visible spectra spectrophotometer. Typically the detection antibody is conjugated to an enzyme such as peroxidase and the protein is detected by the addition of a soluble chromophore peroxidase substrate such as tetramethylbenzidine followed by 1 M sulfuric acid. The test protein concentration is determined by comparison with standard curves. These protocols are detailed in Current Protocols in Molecular Biology, V. 2 Ch. 11 and Antibodies, a Laboratory Manual, Ed Harlow, David Lane, Cold Spring Harbor Laboratory (1988) pp 579-593.

Alternatively, a biochemical assay can be used to detect expression, or accumulation of CMG-2, e.g., by detecting the presence or absence of a band in samples analyzed by polyacrylamide gel electrophoresis; by the presence or absence of a chromatographic peak in samples analyzed by any of the various methods of high performance liquid chromatography, including reverse phase, ion exchange, and gel permeation; by the presence or absence of CMG-2 in analytical capillary electrophoresis chromatography, or any other quantitative or qualitative biochemical technique known in the art.

The immunoassays discussed above involve using antibodies directed against the CMG-2 protein, or against fragments or variants thereof.

### Anti-CMG-2 Antibodies

Anti-CMG-2 antibodies include but are not limited to polyclonal, monoclonal, chimeric, single chain, Fab fragments, and Fab expression library. Various procedures known in the art may be used for the production of polyclonal antibodies to CMG-2 polypeptides or derivative or analog thereof. For the production of antibody, various host animals can be immunized by injection with the antigenic polypeptide, including but not limited to rabbits, mice, rats, sheep, goats, etc.

For preparation of monoclonal antibodies directed toward the CMG-2 polypeptides, any technique that provides for the production of antibody molecules by continuous cell lines in culture may be used. These include but are not limited to the hybridoma technique originally developed by Kohler and Milstein (Nature 1975;256:495-497), as well as the trioma technique, the human B-cell hybridoma technique (Kozbor et al., Immunology Today 1983;4:72; Cote et al., Proc. Natl. Acad. Sci. U.S.A. 1983;80:2026-2030), and the EBV-hybridoma technique to produce human monoclonal antibodies (Cole et al., in Monoclonal Antibodies and Cancer Therapy 1985, Alan R. Liss, Inc., pp. 77-96). In an additional embodiment of the invention, monoclonal antibodies can be produced in germ-free animals (International Patent Publication No. WO89/12690, published 28 Dec, 1989).

According to the invention, techniques described for the production of single chain antibodies (U.S. Pat. Nos. 5,476,786 and 5,132,405 to Huston; U.S. Pat. No. 4,946,778) can be adapted to produce the CMG-2 polypeptide-specific single chain antibodies. Indeed, these genes can be delivered for expression in vivo. An additional embodiment of the invention utilizes the techniques described for the construction of Fab expression libraries (Huse et al., Science 1989;246: 1275-1281) to allow rapid and easy identification of monoclonal Fab fragments with the desired specificity for a CMG-2 polypeptide, or its derivatives, or analogs. Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitation reactions, immunodiffusion assays, in situ inmmunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

### CMG-2 Assays

As described herein, decreased activity or level of CMG-2 is indicative of JHF, ISH, or conditions associated with such disorders, such as osteoporosis and arthritis. In one embodiment one may assess the activity of a CMG-2 protein in a test subject or biological sample taken from the subject and compare it with a control. A decreased activity of the CMG-2 protein in the test subject or biological sample compared with the control is indicative of JHF or ISH, or a condition associated therewith, in the test subject. As described above, a labeled CMG-2 ligand can, for example, be used to detect lack of binding or reduced binding to patient fibroblasts, or the lack of cellular response to ligand binding detected.

The activity of CMG-2 can also be indirectly assayed by evaluating the capability of patient fibroblasts to bind to a laminin substrate or to produce the active form of MMP-2. The nucleic acid-based assays or protein-based assays as described herein may be readily adapted for that purpose. One example of an indirect CMG-2 activity assay is as follows: Cells are plated in serum-free DMEM or other suitable media at a density of about 0.1-10×10⁵ cells per well on laminin and allowed to adhere for 60-90 minutes. After the incubation, the wells are washed and fixed in ethanol for 10 minutes. The remaining bound cells are stained with a suitable staining agent, e.g., 0.5% crystal violet, and washed extensively with water. The fraction of bound cells is then evaluated and compared to control. For example, after crystal violet staining, the well contents can be solubilized with sodium dodecyl sulfate (SDS), and relative adhesion quantified by measuring the absorbance at 540 nm.

An alternative indirect assays for CMG-2 deficiency is to detect fibroblast activation of a gelatinase such as MMP-2 (see Example 4). While the fibroblasts can be attached to a laminin substrate in the presence of serum, lower amounts of active MMP-2 is subsequently secreted in (serum-free) medium. For example, patient and control fibroblasts can be plated on laminin-coated and plastic cell culture dishes in serum-containing media, and allowed to attach and grow for 48 hours. After washing, serum-free media is added, and the cells incubated for a further 24 hours. The supernatant is then harvested, centrifuged to remove debris, and analyzed for active/inactive MMP-2 contents using electrophoresis or another suitable procedure.

### Diagnostic Kits

The present invention further provides kits for the determination of the sequence within the CMG-2 gene in an individual. The kits comprise a means for determining the sequence at the variant positions, and may optionally include data for analysis of mutations. The means for sequence determination may comprise suitable nucleic acid-based and immunological reagents. Preferably, the kits also comprise suitable buffers, control reagents where appropriate, and directions for determining the sequence at a variant position.

Nucleic Acid Based Diagnostic Kits. The invention provides nucleic acid-based methods for detecting genetic variations of CMG-2 in a biological sample. The sequence at particular positions in the CMG-2 gene is determined using any suitable means known in the art, including without limitation one or more of hybridization with specific probes for PCR amplification (e.g., primer pairs selected from SEQ ID NOS: 26 to 51), restriction fragmentation, direct sequencing, SSCP, and other techniques known in the art.

The present invention also provides kits suitable for nucleic acid-based diagnostic applications. In one embodiment, diagnostic kits include the following components: (a) probe DNA: The probe DNA may be pre-labeled; alternatively, the probe DNA may be unlabeled and the ingredients for labeling may be included in the kit in separate containers; (b) hybridization reagents: the kit may also contain other suitably packaged reagents and materials needed for the particular hybridization protocol, including solid-phase matrices, if applicable, and standards. In another embodiment, diagnostic kits include: (a) sequence determination primers: sequencing primers may be pre-labeled or may contain an affinity purification or attachment moiety; and (b) sequence determination reagents: The kit may also contain other suitably packaged reagents and materials needed for the particular sequencing protocol. In one preferred embodiment, the kit comprises a panel of sequencing primers, whose sequences correspond to sequences adjacent to variant positions.

Antibody Based Diagnostic Kits. The invention also provides antibody-based methods for detecting variant (or wild type) CMG-2 proteins in a biological sample. The methods comprise the steps of: (i) contacting a sample with one or more antibody preparations, wherein each of the antibody preparations is specific for variant (or wild type) CMG-2 under conditions in which a stable antigen-antibody complex can form between the antibody and CMG-2 in the sample; and (ii) detecting any antigen-antibody complex formed in step (i) using any suitable means known in the art, wherein the detection of a complex indicates the presence of variant (or wild type) CMG-2.

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labeled immunoassays, such as ELISA assays.

The present invention also provides kits suitable for antibody-based diagnostic applications. Diagnostic kits typically include one or more of the following components: (i) CMG-2-specific antibodies: The antibodies may be pre-labeled; alternatively, the antibody may be unlabeled and the ingredients for labeling may be included in the kit in separate containers, or a secondary, labeled antibody is provided; and (ii) reaction components: The kit may also contain other suitably packaged reagents and materials needed for the particular immunoassay protocol, including solid-phase matrices, if applicable, and standards. The kits may include instructions for conducting the test. Furthermore, in preferred embodiments, the diagnostic kits are adaptable to high-throughput and/or automated operation.

### Therapeutics

Having identified that inactivation or alteration of CMG-2, leading to CMG-2 deficiency, plays a role in the pathology of JHF and ISH, the invention provides a method of treating or preventing these disorders, and/or one or more conditions associated with either one or both of these disorders, in a subject. The term “therapy” or “treatment” means to therapeutically intervene in the development or progression of a disease in a subject showing a symptom of this disease. The term “treatment” also encompasses prevention, which means to prophylactically interfere with a pathological mechanism that results in the disease.

In one embodiment, the method comprises administering an amount of a vector that expresses a gene encoding functional CMG-2 effective to express a functional level of CMG-2 into cells of the subject. More particularly this expression vector is useful for expressing the CMG-2 protein in somatic cell types for human gene therapy. “Gene therapy” refers to transfer of a gene encoding an effector molecule into cells, in this case of the tumor. Gene therapy vectors include, but are not limited to, viral vectors (including retroviruses and DNA viruses), naked DNA vectors, and DNA-transfection agent admixtures. Preferably, a therapeutically effective amount of the vectors are delivered in a pharmaceutically acceptable carrier.

Accordingly, the invention provides a vector, such as a defective virus (particularly a neurotrophic virus) or non-viral vector, that comprises a gene encoding a functional human CMG-2 operatively associated with a regulatory sequence that allows expression of the CMG-2 gene in human target cells in vivo. This regulatory sequence preferably comprises a promoter that provides for a high level of expression of the CMG-2 gene. A pharmaceutical composition for treating or preventing CMG-2 deficiency can be made by combining such a vector and one or more pharmaceutically acceptable carriers. The pharmaceutical composition can be employed to prevent or treat syndromes or conditions associated with CMG-2 deficiency, which method comprises introducing a gene encoding a CMG-2 protein into the mammalian cells, whereby the ability of the mammalian cells to produce functional CMG-2 is restored. The pharmaceutical compositions may also include other biologically active compounds. Vectors suitable for use in CMG-2 gene therapy are described in more detail below.

As an alternative to gene therapy, the invention contemplates preventing or treating CMG-2 deficiency of cells in a subject by administering a therapeutically effective amount of a functional CMG-2 protein, or analogues thereof, to the subject. In yet another embodiment, an agent acting downstream to a dysfunctional CMG-2 protein can be administered to alleviate one or more aspects of the CMG-2 deficiency. The CMG-2 protein CMG-2 analog, or downstream agent, is advantageously formulated in a pharmaceutical composition, with a pharmaceutically acceptable carrier. This substance may be then called active ingredient or therapeutic agent against CMG-2 deficiency. The concentration or amount of the active ingredient depends on the desired dosage and administration regimen, as discussed below. Suitable dose ranges may include from about 0.01 mg/kg to about 100 mg/kg of body weight per day, or may be administered according to a schedule resulting in a therapeutically effective amount being delivered to the subject. This type of treatment is described in more detail below.

The term “therapeutically effective amount” is used herein to mean an amount or dose sufficient to e.g., increase the level of CMG-2 expression and/or activity, or an activity downstream to a CMG-2 protein, e.g., to at least about 10 percent, preferably to at least about 50 percent, more preferably to at least about 90 percent, most preferably to at least about 95%, and optimally to about 100% of a control level. Preferably, a therapeutically effective amount can ameliorate or present a clinically significant improvement in at least one CMG-2 activity in the subject. Alternatively, a therapeutically effective amount is sufficient to cause an improvement in a clinically significant condition in the host, e.g., to improve osteoporosis, arthritis, or another condition associated with JHF and/or ISH.

A composition comprising “A” (where “A” is a single protein, DNA molecule, vector, recombinant host cell, etc.) is substantially free of “B” (where “B” comprises one or more contaminating proteins, DNA molecules, vectors, etc.) when at least about 75% by weight of the proteins, DNA, vectors (depending on the category of species to which A and B belong) in the composition is “A”. Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

According to the invention, the pharmaceutical composition of the invention can be introduced parenterally, transmucosally, e.g., orally (per os), nasally, or rectally, or transdermally. Parental routes include intravenous, intra-arteriole, intra-muscular, intradermal, subcutaneous, intraperitoneal, intraventricular, and intracranial administration. The pharmaceutical compositions may also be added to a retained physiological fluid such as blood or synovial fluid. In another embodiment, the active ingredient can be delivered in a vesicle, in particular a liposome (see Langer, Science 1990;249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.). In yet another embodiment, the therapeutic compound can be delivered in a controlled release system. For example, a polypeptide may be administered using intravenous infusion with a continuous pump, in a polymer matrix such as poly-lactic/glutamic acid (PLGA), a pellet containing a mixture of cholesterol and the active ingredient (SilasticRTM; Dow Corning, Midland, Mich.; see U.S. Pat. No. 5,554,601) implanted subcutaneously, an implantable osmotic pump, a transdermal patch, liposomes, or other modes of administration.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin. Such methods, including routes of administration and dose, are well known in the art.

### Gene Therapy Vectors

As discussed above, a vector is any means for the transfer of a nucleic acid according to the invention into a host cell. Preferred vectors for transient expression are viral vectors, such as retroviruses, herpes viruses, adenoviruses and adeno-associated viruses. Thus, a gene encoding a functional CMG-2 protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication No. WO 95/28494.

The DNA-based and viral vectors described above for use in expressing wild-type or variant CMG-2 polypeptides in vitro can also be used for in vivo or ex vivo targeting and therapy procedures. Other suitable viral vectors include defective herpes simplex virus, which has been shown to be effective for delivery of genes, particularly to cells of the CNS (see, e.g., Belloni et al., Human Gene Therapy 1996;7:2015-24). Recombinant defective adenoviruses have also been used for transferring foreign genes into cells, particularly for gene therapy of tumors, and for delivery of therapeutic genes to cells of the central nervous system. For example, PCT Publication Nos. WO 94/08026 and WO 94/08026 describe recombinant adenovirus vectors for the transfer of foreign genes into the central nervous system (CNS). Other examples of gene delivery to the CNS include the following: French Publication No. FR2717824 discloses adenoviruses containing DNA from glial derived neutrophilic factors, which infected nerve cells very efficiently; various publications describe adenoviral vectors that express glial maturation factor (FR2717497), brain derived neurotropic factor (FR2717496) and acidic fibroblast growth factor (FR2717495); PCT Publication No. WO 95/26409 describes adenoviruses containing the DNA sequence for basic fibroblast growth factor to infect cells directly or via implants to treat neurological disorders; PCT Publication No. WO 96/00790 describes adenoviruses containing DNA encoding superoxide dismutase (SOD) to treat neurodegenerative diseases and excessive SOD expression; and PCT Publication No. WO 96/01902 describes adenoviruses expressing nitric oxide synthase for gene therapy where angiogenesis is required for treating disorders of the CNS.

Adenoviruses can be genetically modified to reduce the levels of viral gene transcription and expression, including adenoviruses defective in the E1 and E4 regions (PCT Publication No. WO 96/22378) and adenoviruses with an inactivated E1 region but also with altered genomic organization reducing the number of viable viral particles produced if recombination occurs with the host genome (PCT Publication No. WO 96/13596). PCT Publication No. WO 96/10088 describes defective adenoviruses with an inactivated IVa2 gene. PCT Publication No. WO 95/02697 describes an adenovirus defective in regions E1 and E2, E4, or L1-L5.

In another embodiment, a gene can be introduced using a combined virus, also termed plasmovirus (Genopoietic, France) vector system. Plasmovirus systems permit one cycle of infectious virus formation in infected host cells. In these systems, a complementing gene(s) for defective viral genome sequences and the defective viral sequences are both provided to target cells in vivo or in vitro. The primary infected cells produce infectious, defective virus. By permitting one cycle of infectious defective virus formation in infected cells, plasmovirus technology amplifies gene delivery in vitro and, particularly, in vivo. This cycle of infectious virus formation in situ permits wider infection of tumor cells in a tumor, thus enhancing the anti-tumor effect and reducing reliance on the bystander effect. See PCT Publication Nos. WO 95/22617, WO 95/26411, WO 96/39036, WO 97/19182.

Alternatively, the vector can be introduced in vivo by lipofection, as naked DNA, as a naked DNA plasmid, or with other transfection facilitating agents (peptides, polymers, etc.), according to the methods described for expressing CMG-2 polypeptides (above). Gene correction in vitro, optionally for later administration of cells in vivo, may also be achieved by various techniques, including chimeraplasty (Tagalakis et al., J Biol Chem 2001;276(16):13226-30; Ken et al., Semin Liver Dis 1999;19(1):93-104). This technique is based on the observation that oligonucleotides containing complementary RNA/DNA hybrid regions are more active than duplex DNA in homologous pairing reactions in vitro. The chimeric molecules are designed with a homologous targeting sequence comprised of a DNA region flanked by blocks of 2′-O-methyl RNA residues (the chimeric strand), its complementary all-DNA strand, thymidine hairpin caps, a single-strand break, and a double-stranded clamp region. The oligonucleotide can align in perfect register with a genomic target except for the designed single base pair mismatch, which is recognized and corrected by harnessing the cell's endogenous DNA repair system.

Other possible techniques include transposon technology, successfully reported for the nonhomologous insertion of foreign genes into genomes of adult mammals using naked DNA (Yart et al., Nat Genet 2000;251(1):35-41). Linear DNA concatamers provide another approach for achieving expression of a transgene in vivo (Chen et al., Mol Ther 2001;3(3):403-10).

### CMG-2 Protein Therapy

As described above, an effective amount of a functional CMG-2 protein can be administered to a subject to prevent or treat CMG-2 deficiency. Pharmaceutical compositions comprising a CMG-2 protein as an active ingredient, with a pharmaceutically acceptable carrier, are thus encompassed by the invention. The use of analogues, derivatives or mimetics of the CMG-2 protein as the active ingredient, are also contemplated.

In one embodiment, the active ingredient is designed so that it is less prone to proteolytic enzyme digestion by, e.g., such as enzymes of the digestive tract. In another embodiment, a CMG-2 protein is modified by conjugation to a translocation peptide sequence. Specifically, peptide sequences have been identified that mediate membrane transport, thus providing for delivery of polypeptides to the cytoplasm. For example, translocation peptides can be derived from the antennapedia homeodomain helix 3 to generate membrane transport vectors, such as penetratin (PCT Publication WO 00/29427; see also Fischer et al., J. Pept. Res. 2000;55:163-72; DeRossi et al., Trends in Cell Biol 1998;8:84-7; Brugidou et al., Biochem Biophys Res Comm 1995;214:685-93). Protein transduction domains, which include the antennapedia domain and the HIV TAT domain (see Vives et al., J Biol Chem 1997;272:16010-17), posses a characteristic positive charge, which led to the development of cationic 12-mer peptides that can be used to transfer therapeutic proteins and DNA into cells (Mi et al., Mol Therapy 2000;2:339-47). Accordingly, therapeutic polypeptides are generated by creating fusion proteins or polypeptide conjugates combining a translocation peptide sequence with a therapeutically functional sequence. For example, p21WAF1-derived peptides linked to a translocation peptide inhibited ovarian tumor cell line growth (Bonfanti et al., Cancer Res 1997;57:1442-1446). These constructs yield more stable, drug-like, polypeptides able to penetrate cells and effect a therapeutic outcome. These constructs can also form the basis for rational drug design approaches.

In addition, complexes or conjugates containing tetrameric streptavidin, e.g., including a biotinylated protein, translocate into the cytoplasmic efficiently with preservation of protein function. A preferred such construct employs a Protein A-streptavidin fusion protein, which can bind a targeting antibody and the active protein, which can be biotinylated (see, e.g., U.S. Pat. No. 5,328,985; Sano and Cantor, Bio/Technology 1991;9:1378-81; Ohno et al., Biochem Mol Med 1996;58:227-33; Yu et al., DNA and Cell Biol. 2000;19:383-8).

### Screening Methods

As described in Example 4, the addition of serum to CMG-2 deficient patient fibroblasts, which are unable to bind to laminin, restored laminin-binding capability to the cells. Serum thus contains one or more agents or drug candidates that can be used in a pharmaceutical composition to treat or prevent JHF, ISH, and/or one or more conditions associated with these disorders. This agent or agents can be identified by, e.g., fractionating serum and testing which fraction restores laminin-binding and/or MMP-2 processing of cells expressing variant CMG-2. Serum or serum-fractions can also be treated to, for example, heat-inactivate proteins to test if a protein or polypeptide is responsible for the restoring effect.

Another manner of identifying suitable drug candidates is via screening of test substances. A “test substance” is a chemically defined compound or mixture of compounds (as in the case of a natural extract or tissue culture supernatant), whose ability to overcome CMG-2 activity may be defined by various assays. A “test substance” is also referred to as a “candidate drug” or “drug candidate” herein. Test substances may be screened from large libraries of synthetic or natural compounds, or isolated from a biological fluid such as serum. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from, e.g., Pan Laboratories (Bothell, Wash.) or MycoSearch (NC), or are readily producible. Additionally, natural and synthetically produced libraries and compounds are readily modified through conventional chemical, physical, and biochemical means (Blondelle et al., TIBTech 1996; 14:60).

In one embodiment, the cell assay described in Example 4 is applied to evaluate whether a test substance can be used for treating or preventing CMG-2 deficiency. For example, cells in which a CMG-2 gene is inactivated, or dermal fibroblasts isolated from a JHF or ISH patient, can be contacted with a candidate compound and cell-binding to laminin substrate thereafter evaluated. Such an assay will identify CMG-2 substitutes, i.e., compounds that can alleviate for the CMG-2 deficiency. Candidate compounds that lead to CMG-2 expression, improved CMG-2 activity, or laminin-binding are selected.

Any screening technique known in the art can be used to screen for drug candidates. The present invention contemplates screens for synthetic small molecules as well as screens for natural molecules, using synthetic libraries or natural products libraries.

One approach uses recombinant bacteriophages to produce large libraries. Using the “phage method” (Scott and Smith, Science, 1990, 249:386-390; Cwirla, et al., Proc Natl Acad Sci USA 1990;87:6378-6382; Devlin et al., Science 1990;49:404-406), very large libraries can be constructed (10⁶-10⁸ chemical entities). A second approach uses primarily chemical methods, of which the Geysen method (Geysen et al., Molecular Immunology 1986;23:709-715; Geysen et al., J Immunol Meth 1987;102:259-274; and the method of Fodor et al. (Science 1991;251:767-773) are examples. Furka et al. (14th International Congress of Biochemistry, 1988, Volume #5, Abstract FR:013; Furka, Int. J. Peptide Protein Res 1991;37:487-493), and U.S. Pat. Nos. 4,631,211 and 5,010,175 describe methods to produce a mixture of peptides that can be tested for their capability in alleviating one or more aspects of CMG-2 activity.

In another aspect, synthetic libraries (Needels et al., Proc Natl Acad Sci USA 1993;90:10700-4; Ohlmeyer et al., Proc Natl Acad Sci USA 1993;90:10922-10926; PCT Publication Nos. WO 92/00252 and WO 94/28028) and the like can be used to screen for CMG-2 ligands or compounds acting in a CMG-2 pathway according to the present invention.

When screening for compounds affecting CMG-2 expression or activity in the presence of test substances, various reporter gene assays can be used. For example, a green fluorescent protein expression assay permits evaluation of CMG-2 expression and/or activity. GFP can be modified to produce proteins that remain functional but have different fluorescent properties, including different excitation and emission spectra (U.S. Pat. No. 5,625,048 and PCT Publication No. WO 98/06737); an enzyme recognition site (PCT Publication No. WO 96/23898); increased intensity compared to the parent proteins (PCT Publication No. WO 97/11094); higher levels of expression in mammalian cells (PCT Publication No. WO 97/26633); twenty times greater fluorescence intensity than wild-type GFP (PCT Publication No. WO 97/42320); and mutants excitable with blue and white light (PCT Publication No. WO 98/21355). Other reporter genes include luciferase, β-galactosidase (β-gal or lac-Z), chloramphenicol transferase (CAT), horseradish peroxidase, and alkaline phosphatase. In addition, expression of almost any protein can be detected using a specific antibody.

Selected agents may be modified to enhance efficacy, stability, pharmaceutical compatibility, and the like. Structural identification of an agent may be used to identify, generate, or screen additional agents. For example, where peptide agents are identified, they may be modified in a variety of ways, e.g. to enhance their proteolytic stability.

### EXAMPLES

The invention is illustrated in the following examples, which are provided by way of illustration and are not intended to be limiting.

### Example 1

### Identifying CMG-2 and Mutations Associated with JHF and ISH

The JHF disease gene was recently localized to chromosome 4q21 using a positional cloning approach (Rahman et al., Am J Hum Genet. 2002;71:975-980). The 5.3 cM/6.9 Mb locus is bounded by microsatellite marker D4S2393 centromerically and D4S395 telomerically (Rahman et al., 2002, supra; Kong et al., Nat Genet 2002;31:241-7). In an attempt to further refine the locus and investigate the possibility that these clinically overlapping autosomal recessive disorders, JHF and ISH, are indeed allelic, four unrelated families with established clinical diagnoses and features consistent with these syndromes were first ascertained. Various features of the patients are provided in Table 3. In one affected individual in family JHF1, radiological features included diffuse osteopenia, narrowing of interarticular spaces, and multiple subluxations and contractures in both hands, as well as a marked narrowing of joint space and profound osteopenia in the knees.

TABLE 3
Comparison Of Features Of Patients With Juvenile Hyaline
Fibromatosis And Infantile Systemic Hyalinosis.

| Features | JHF1 | JHF2 | ISH1 | ISH2 |
|---|---|---|---|---|
| Consanguinous | + | + | + | − |
| Ethnic origin | Turkish | African | Turkish | Swiss |
| | | American | | |
| Skin | | | | |
| Multiple subcutaneous tumors | + | + | − | + |
| Thickened firm skin | − | − | + | + |
| Pearly nodules | + | + | − | + |
| Perianal granulomas | + | − | − | + |
| Gingiva | | | | |
| Gingival hypertrophy | − | + | − | + |
| Gingival fibromatosis | + | ? | − | ? |
| Skeletal findings | | | | |
| Joint contractures | + | − | + | + |
| Restricted movement of joints | + | − | + | + |
| Painful joints | + | − | + | + |
| Osteoporosis | + | ? | + | + |
| Osteopenia | + | ? | + | + |
| Growth | | | | |
| Failure to thrive | − | − | + | + |
| Stunted growth | + | − | − | + |
| Facial features | | | | |
| Coarse face | + | + | − | + |
| Narrow face | − | − | + | + |
| Large low-set dysplastic ears | − | − | + | + |
| Micellaneous | | | | |
| Early death | − | − | + | − |
| Recurrent infections | +/− | − | + | − |
| Histology | | | | |
| Accumulation of material | | | | |
| In skin | − | + | + | + |
| In articular soft tissues | ? | − | ? | + |

Note:
Family ISH 2 has been previously described by Stucki et al. 2001, supra.

Using a dense set of microsatellite markers spanning the linked region, available family members were all haplotyped to look for regions which were homozygous-by-descent. After informed consent and Institutional Review Board approval from the corresponding institutions, blood samples were collected from family members and genomic DNA was isolated. Haplotype analysis was performed using 8 fluorescently labelled microsatellite markers (D4S2393; D4S2947; D4S2964; D4S3243; D4S2922; D4S2932; D4S395). Markers were amplified by PCR using standard protocols and products were run on an ABI3100 Genetic Analyzer (Applied Biosystems, Foster City, Calif.) and electropherograms analysed by the ABI Genescan and Genotyper software packages (Perkin Elmer), as previously described (Heath et al., Am J Hum Genet 2001;69:1033-45). The following primer sequence were used:

SED ID NO:

| | | |
|---|---|---|
| D4S2393 | | |
| Left Primer: | GTGAGCTTTTAACTTGGCCA | 52 |
| Right Primer: | CCTTGTCTTCCTTACAAACCC | 53 |
| Distance: | 103-115 bps | |
| D4S2947 | | |
| Left Primer: | CCTAGCCAATAGAGACCGTG | 54 |
| Right Primer: | AGAGAGATCCCTCATCCCT | 55 |
| Distance: | 229-247 bps | |
| D4S2964 | | |
| Left Primer: | AAGCTAAGACCCAACTTCTTT | 56 |
| Right Primer: | TCATGCAATCCACACAG | 57 |
| Distance: | 159-197 bps | |
| D4S3243 | | |
| Left Primer: | AATCCAGTAAATGAAATAGTCATCA | 58 |
| Right Primer: | ATAAGCCAAACATGATGGGA | 59 |
| Distance: | 170-171 bps | |
| D4S2922 | | |
| Left Primer: | CATGTTTCCACTCCAGTTCT | 60 |
| Right Primer: | ATAAAGGGCAGTTAGGGATG | 61 |
| Distance: | 258-268 bps | |
| D4S2932 | | |
| Left Primer: | GAGCAAAACTCTGTCTCAAAAATAA | 62 |
| Right Primer: | GGCTTACTTGGAAAGGTCTCTT | 63 |
| Distance: | 209-221 bps | |
| D4S3088 | | |
| Left Primer: | GTCTCACCCTGAAAGGGATT | 64 |
| Right Primer: | GGTTACAGGACCACAAGTGC | 65 |
| Distance: | 238 bps | |
| D4S395 | | |
| Left Primer: | TACTCCAGCCTGGATGACAG | 66 |
| Right Primer: | TGTTCCATAACAAGCACGTT | 67 |
| Distance: | 113-137 bps | |

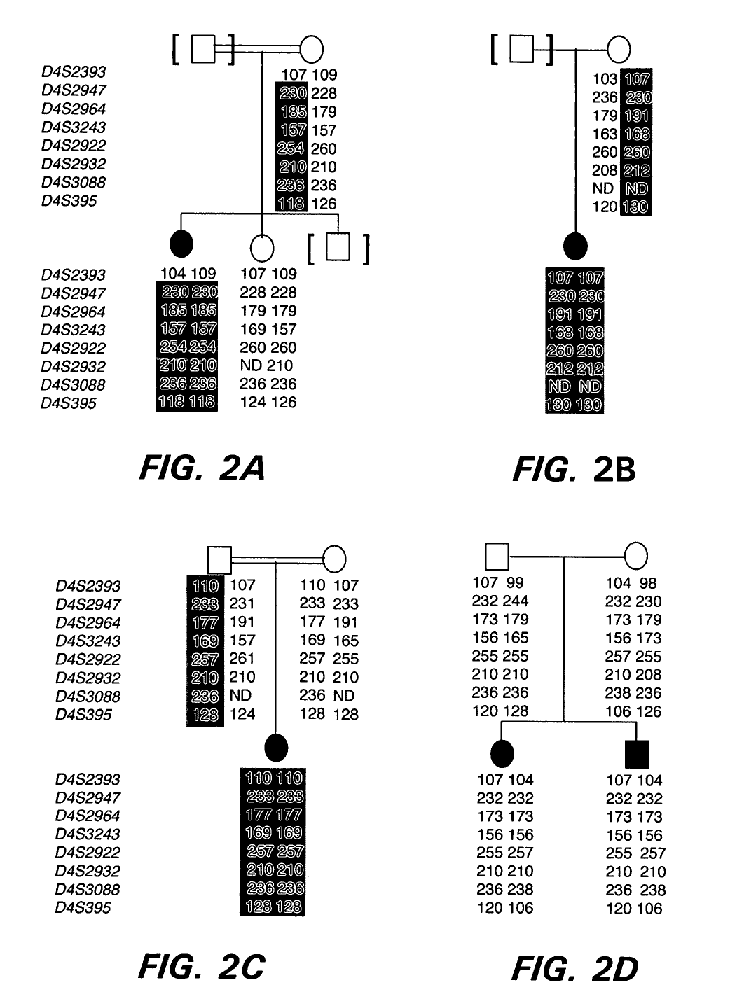

Probands in families ISH1 and JHF2 were homoallelic for all 8 markers which, while consistent with the previous linkage report, did not further narrow the region (FIG. 2). Support for the originally defined centromeric border of the JHF locus was provided by members of the remaining two families. The centromeric boundary of the region was confirmed by non-homozygosity of marker D4S2393 in the JHF1 affected individual, wherein all other tested markers were homoallelic. Interestingly, while the Family ISH2 affected haplotypes suggested a potential narrowing of the distal boundary of the region, as demonstrated by homozygosity of three contiguous markers, D4S2947, D4S2964 and D4S3243, it could not be ruled that this merely reflected “identity-by-state.” Because of this, the candidate gene interval could not be conclusively narrowed. This caution was found to be supported by DNA sequence analysis (see below).

Inspection of genes in the JHF/ISH common region revealed a number of possible disease gene candidates including bone morphogenetic protein 3 (BMP-3), fibroblast growth factor-5 (FGF-5) and capillary morphogenesis protein 2 (CMG-2). Of these, the capillary morphogenesis gene-2 (CMG-2), was immediately attractive because of its expression in endothelial cells and suggested role in binding extracellular matrix proteins, including laminin and collagen IV, by virtue of its von Willebrand factor A (VWFA)-like domain (Bell et al., J. Cell Sci. 2001;114:2755-2773). In addition, the phenotypes of previously reported murine knockouts of BMP3 and FGF5 genes were not consistent with either JHF or ISH (Herbert et al., Cell 1994;78: 1017-1025; Daluiski et al., Nat Genet 2001;27:84-8).

While CMG-2 was originally identified on the basis of its upregulation in endothelial cells induced to undergo capillary formation (Bell et al., J Cell Sci 2001; 114:2755-2773), the physiologic role of CMG-2 is unknown. Interestingly, CMG-2 not only possesses a protein sequence similarity to the tumor endothelial marker 8 (TEM8) gene, a cell-surface receptor which may play a role in neovascularization and is also the human anthrax toxin receptor (ATR), but CMG-2 was also recently shown to function as the second known human ATR (Scobie et al., Proc Natl Acad Sci USA 2003; 100:5170-5174). The predicted topology of CMG-2 is similar to ATR/TEM8 in that they both have a signal peptide, type 1 transmembrane region and, within the VWFA or I domain, share 60% identity (FIG. 3), (Bell et al., J Cell Sci 2001; 114:2755-2773).

A first study was therefore directed to determine whether CMG-2 mutations could result in JHF and ISH. First, all human and non-human EST and mRNA data and gene prediction output (UCSC Genome Browser; November 2002 and April 2003 assembly dates) were analyzed to identify possible coding regions since several isoforms had been predicted (Scobie et al., Proc Natl Acad Sci USA 2003;100:5170-5174).

From this combined information, primer pairs were designed to amplify all 17 predicted exons and intron/exon boundaries. The CMG-2-488 isoform was used. This isoform is conserved with the originally cloned CMG-2-386 isoform (Bell et al., J Cell Sci 2001;114:2755-2773) but includes an inserted 100 amino acid membrane-proximal region between the VWA-like domain and transmembrane region and 12 alternative amino acids at the C-terminal (FIG. 3; SEQ ID NO:3). PCR products were sequenced in both directions using the ABI Bigdye terminator sequencing kit (Perkin Elmer) and data was analyzed using Sequencher 4.1 (GeneCodes).

CMG-2 mutations were identified in all affected individuals and these were predicted to either truncate or functionally disrupt the wild type protein. None of the mutations identified in any of the families were present in the genomic DNA isolated from 50 unrelated control subjects (100 chromosomes). The mutations and their locations are identified in Tables 1A and 1B, and are further discussed in Example 2.

### Example 2

### Structural and Functional Implications of Identified Mutations

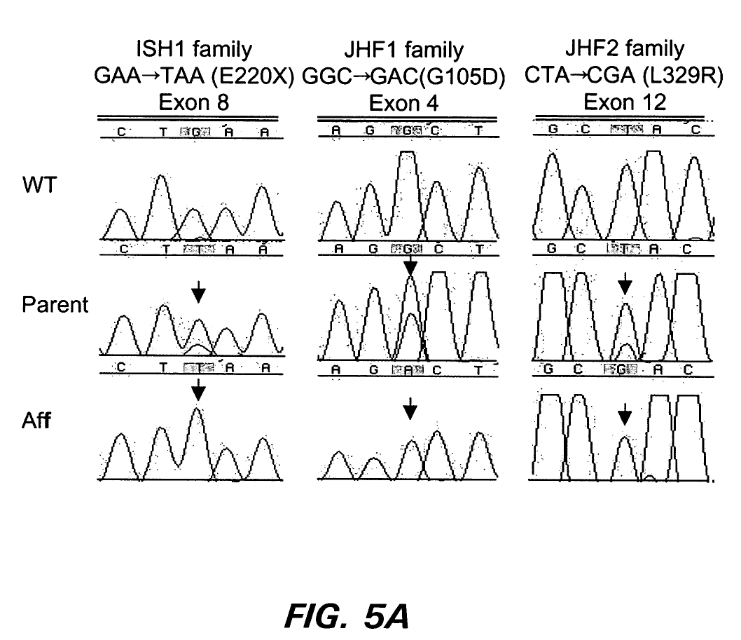

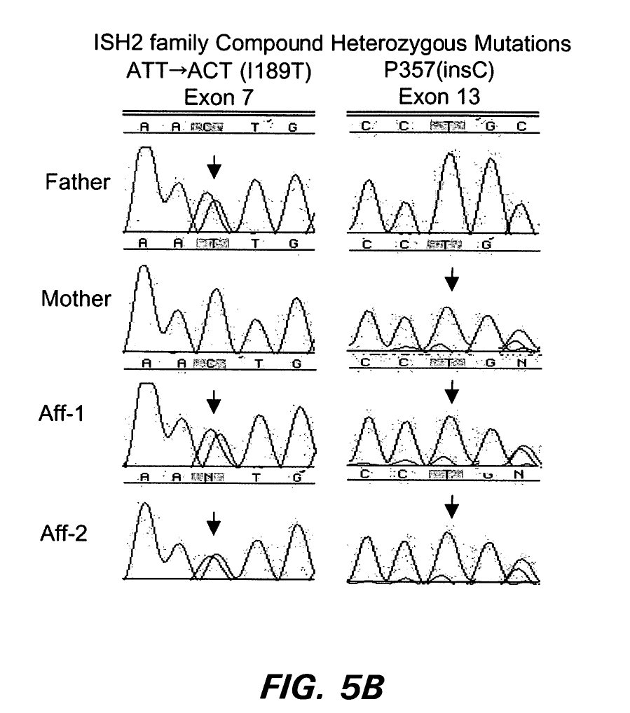

E220X: In family ISH1, the affected individual was found to be homoallelic for a nonsense mutation a GAA→TAA transversion in codon 220 of exon 8 (E220X), (FIG. 5A). This mutation predicts the loss of the majority of the wild type protein, including the transmembrane and cytosolic domains (FIG. 3).

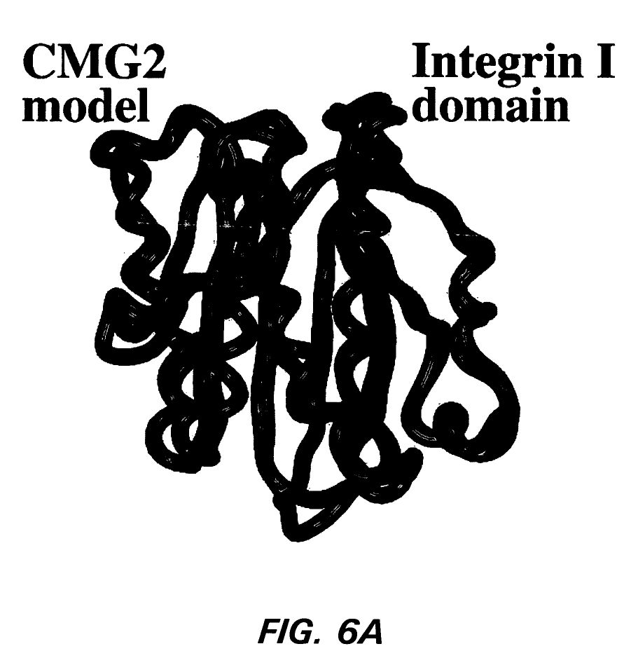

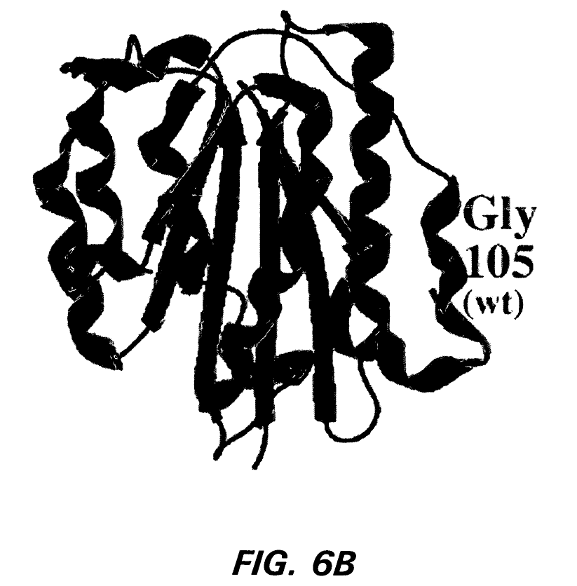

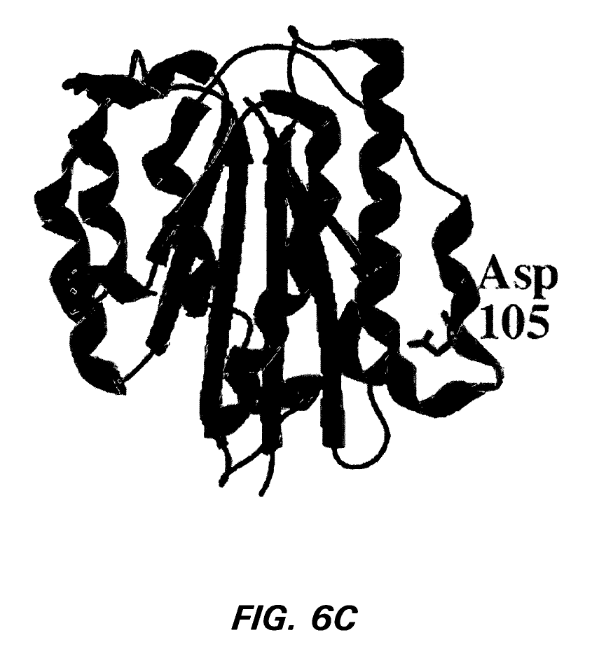

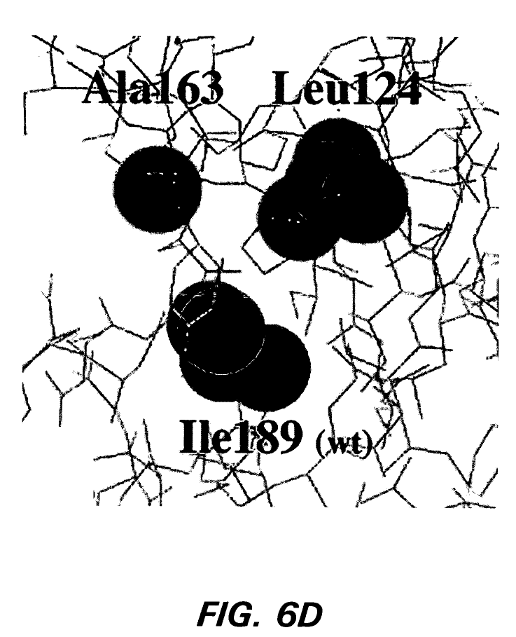

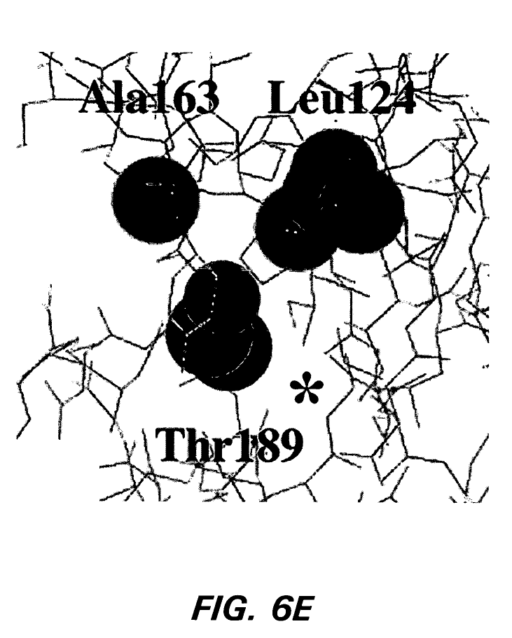

The possible structure-function effects of patient mutations were explored by identifying an appropriate model template. Based on sequence analysis that demonstrated 48% homology, chain A of the Alpha-X Beta2 Integrin I Domain (PDB accession number 1N3Y) was chosen as a template since the structure was solved by X-ray diffraction to atomic resolution (FIG. 6A). The 1N3Y sequence of 198 amino acid residues is as follows (SEQ ID NO:63):

| | |
|---|---|
| 1 | GSHMASRQEQ DIVFLIDGSG SISSRNFATM MNFVRAVISQ FQRPSTQFSL |
| 51 | MQFSNKFQTH FTFEEFRRSS NPLSLLASVH QLQGFTYTAT AIQNVVHRLF |
| 101 | HASYGARRDA AKILIVITDG KKEGDSLDYK DVIPMADAAG IIRYAIGVGL |
| 151 | AFQNRNSWKE LNDIASKPSQ EHIFKVEDFD ALKDIQNQLK EKIFAIEG |

Non-conserved residues from this domain were mutated in silico to the corresponding CMG-2 sequences using the program O (Jones et al., Acta Crystallogr 1991;47:110-119). The CMG-2 model energy was minimized and the effect of mutations on energy minimization, surface accessibility, interatomic distances and potential atomic interactions, was evaluated using the Molecular Operating Environment suite of programs (CCG, Montreal Canada).

G105D: DNA sequence analysis of Family JHF1 determined the presence of a homozygous change in codon 105 of exon 4, a GGC→GAC transition, which predicted the replacement of a glycine by an aspartate (G105D) in the VWFA-like domain (FIG. 3; FIG. 5A). VWA domains are found in a number of ECM proteins including integrins, some collagens and the matrilins (Hohenester and Engel, Matrix Biol 2002;2: 115-128; Whittaker and Hynes, Mol Biol Cell 2002;10:3369-3387). Indeed, mutations in the VWFA domain of the matrilin-3 protein have previously been found to result in an osteochondrodysplasia, multiple epiphyseal dysplasia (MIM 607078), (Chapman et al., Nat Genet 2000;28:393-6). While this domain is involved in ligand-recognition in non-ECM molecules, little is known about its role in ECM molecule function (Hohnester and Engel, Matrix Biol 2001;2:115-128). Structure alignment of the G105D mutation showed that the wild type glycine residue maps to the carboxy-terminal end of an alpha-helix containing the Schellman motif (FIGS. 6B AND 6C), (Aurora and Rose, Protein Sci. 1991 ;7:21-38). Therefore, the replacement of glycine by aspartate, a nonconserved acidic residue, could destabilize the critical helical “cap” of this secondary structure motif which could result in the mutation's pathogenicity.

L329R: In family JHF2, we detected a homoallelic mutation in codon 329 of exon 12, a CTA→CGA transversion (FIG. 3; FIG. 5A). Significantly, this is predicted to result in the non-conserved replacement of a leucine residue by an arginine (L329R) within the transmembrane domain (FIG. 3). This change from hydrophobic to charged amino acid alters the calculated hydropathy and charge profile of the transmembrane (TM) domain. Regarding the pathophysiologic role of this mutation, by analogy with other TM protein regions, the altered CMG-2 leucine is in the center of a stretch of five contiguous leucines within the TM region and thus could effect problems in cell surface expression, affinity for other TM regions or for ligand binding and subsequent signaling (Scott et al., Thromb Haemost 1998;4:546-550). Alternatively, if CMG-2 is in a monomeric state, the introduction of an aspartate may cause receptor aggregation by placing a buried charge within the membrane.

P357insC: Surprisingly, the affected individuals in Family ISH2 were found to be compound heterozygotes for CMG-2 disease mutations. In accord with the identified germline mutations, RNA isolated and directly sequenced from cultured fibroblasts confirmed the existence of two transcripts. First, each individual possessed a 1 bp C nucleotide insertion in codon 357 of exon 13, predicting a frameshift mutation, incorporation of a novel 12 amino acid carboxy-tail and a premature downstream stop codon (TGA; P357insC; SEQ ID NO:8). The P357insC truncation results in the loss of the terminal 132 amino acid residues comprising the cytoplasmic domain (FIG. 3; FIG. 5B). While no functional roles have yet been defined for this region, it would be expected that this truncated cytoplasmic domain is normally an important modulator in relaying signals across the plasma membrane. In fact, two Wiskott-Aldrich syndrome protein (WASP)-homology1 (WH1) domains are present in this region (Bell et al.2001, supra), and therefore loss of both of these domains could result in loss of actin cytoskeleton interaction.

I189T: The second mutation, in codon 189 of exon 7, was predicted to replace an isoleucine with a polar threonine residue (1189T) (FIG. 3; FIG. 5B). For the 1189T mutation, the larger isoleucine hydrophobic side chain is replaced by a threonine, creating a smaller polar residue towards the interior of the protein (FIG. 6D and 6E). We calculated that the I189T mutation results in the production of an internal 40 cubic Angstrom cavity within the protein (FIG. 6E, asterisk), thus completely altering the hydrophobic forces within the protein (Takano et al., Protein Eng. 2003; 1:5-9).

### Example 3

### Recombinant Expression of Mutant CMG-2

To examine the effects of patient-derived mutations on protein synthesis, we generated cDNAs encoding all identified CMG-2 protein mutants by site-directed mutagenesis. The patient mutations were introduced using the Quick-Change site-directed mutagenesis kit according to the manufacturer's protocol (Stratagene) and all constructs were sequenced in both orientations prior to transfection into HEK 293 cells. Western blots were performed on cell lysates using an affinity purified rabbit polyclonal antibody directed to the CMG-2 VWF A domain (Bell et al., J. Cell Sci. 2001;114:2755-2773).

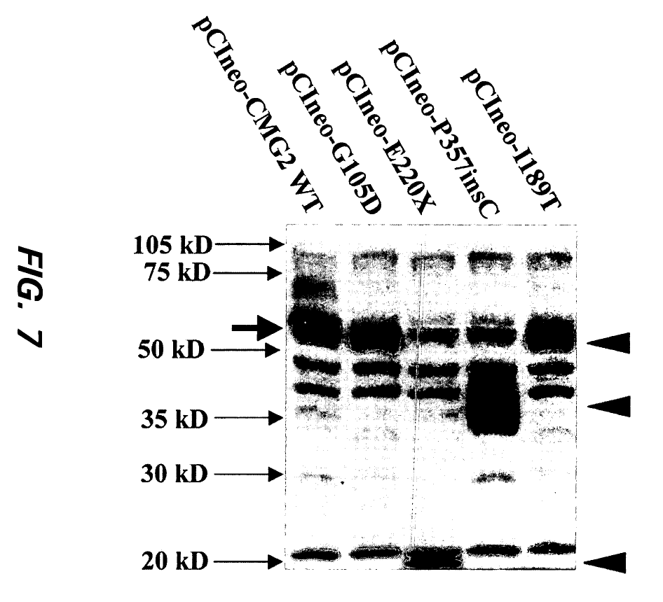

As shown in FIG. 7, all of the patient-derived CMG-2 cDNA constructs are expressed and translated. Most notably, whereas wild type CMG-2 protein (pCIneo-CMG-2-WT; upper arrowhead in the figure) migrates at about 55 kDa, the E220X and P357insC mutations resulted in products migrating at about 20 kDa and about 35-40 kDa, respectively. The MW of both of these proteins was consistent with the size of the predicted truncation products. Interestingly, the P357insC directed protein results in multiple tightly migrating bands, which would suggest either post-translational modification differences, possibly glycosylation, or that the mutated protein is unstable and being degraded.

### Example 4

### Lack of Laminin Binding and MMP-2 Activation of Patient Fibroblasts

This Example shows that altered CMG-2/laminin interaction could play a role in disease pathogenesis. The vWFA domain of the CMG-2 protein produced as a recombinant protein in bacteria was previously shown to bind both laminin and type IV collagen (Bell et al., J. Cell Sci. 2001; 114:2755-2773). Along with its homology to Alpha-X Beta2 Integrin I Domain, this binding pattern is suggestive of a potential role for CMG-2 in the modulation of cell-matrix, cell-cell interactions possibly in the capacity of a matrix receptor. In this study, the ability of JHF and ISH patient fibroblasts to attach, spread, and grow were investigated on a variety of matrices.

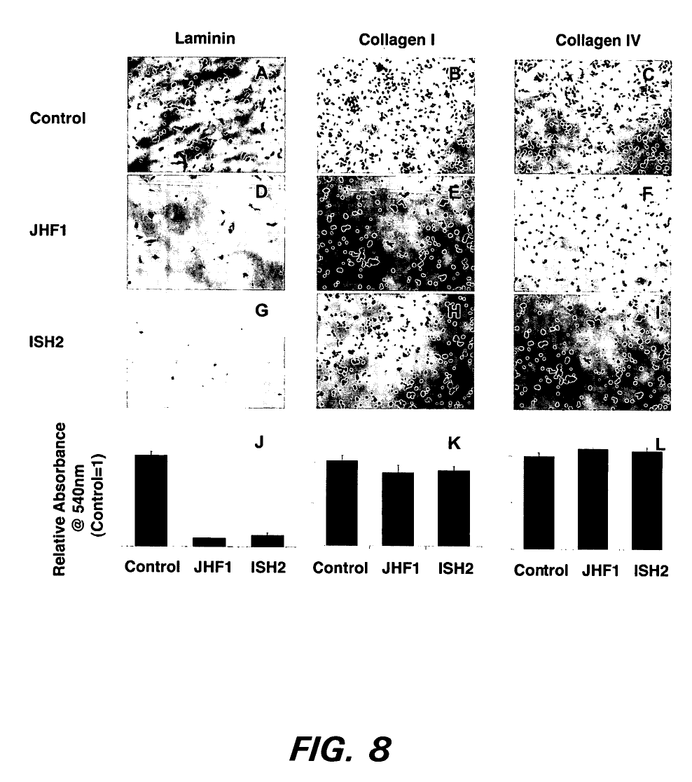

Primary dermal fibroblasts from patient JHF1 and ISH2 were plated on laminin, collagen I and collagen IV containing tissue culture plates (BD Biosciences) and the relative adhesion was quantified (Ellerbroek et al., J. Biol. Chem. 2001;276:24833-24842). Briefly, the following procedure was used to determine extracellular matrix (ECM) binding of patient and control dermal fibroblasts: Cells were plated in serum-free DMEM at a density of 1×10⁵ cells on laminin, collagen I or collagen IV and were allowed to adhere for 75 minutes. After the incubation, wells were washed 3 times with phosphate buffered saline (PBS) and fixed in ethanol for 10 minutes. The remaining bound cells were stained with 0.5% crystal violet for 20 min, washed extensively with water and solubilized with 800 μL 1% sodium dodecyl sulfate (SDS). The relative adhesion was then quantified by measuring the absorbance at 540 nm. This study showed that JHF and ISH fibroblasts were unable to adhere or attach themselves to a laminin matrix (FIGS. 8A, 8D, 8G, and 8J), while no measurable differences were noted for attachment to collagen types I and IV (FIGS. 8B, 8C, 8E, 8F, 8H, 8I, 8K, 8L).

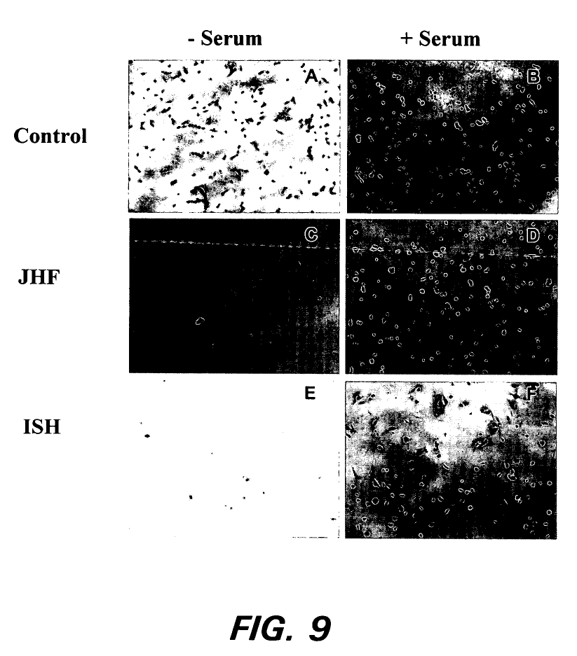

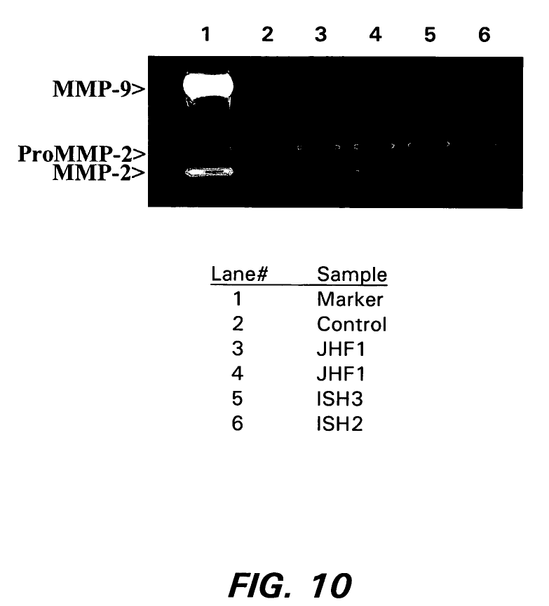

It was found, however, that the CMG-2 deficient fibroblast laminin-binding defect could be corrected with serum. As depicted in FIG. 9A-9F, the addition of 5% bovine serum to cultured CMG-2 deficient fibroblasts, the fibroblasts derived from patients with JHF or the more severe ISH, corrected their previous inability to bind to laminin.

It was also found that CMG-2 deficient cells grown on laminin had an upregulated overall production of MMP-2 expression but lost their ability to activate MMP-2. While normally a rich source of active MMP-2, supernatant from CMG-2 deficient fibroblasts grown in serum-free media showed the presence of the inactive pro-form when assayed by zymography. Briefly, the following procedure was used to detect gelatinases (MMP-2), from patient fibroblasts plated on laminin, by SDS-PAGE: Patient and control fibroblasts (1×10⁶/well) were plated on laminin-coated and plastic cell culture dishes in serum-containing media, and incubated for 48 hours. After washing, serum-free media was added, and the cells incubated for a further 24 hours. The conditioned supernatants of fibroblasts (serum-free media) were harvested and centrifuged for 10 minutes at 14,000 rpm for removal of cell debris. An equal volume of 2×SDS sample buffer was added to each supernatant (note—samples were not boiled). The samples were then loaded onto 10% acrylamide gelatin gels (from Invitrogen) and electrophoresis conducted at 15 mA/gel. The gels were washed three times (20 min/cycle) with H₂O containing 2.5% Triton X-100 at room temperature, and incubated in 200 ml of activation buffer (Final: 10 mM Tris-HCl (pH 7.5) containing 1.25% Triton X-100, 5 mM CaCl₂, 1 μM ZnCl₂) overnight at 37° C. The gels were then stained with Coomassie blue for 2 to 3 hours, and destained with methanol:acetic acid:water (8:1:1) for 30 minutes to 1 hour. Clear zones in the gels indicated the presence of proteinase with gelatinolytic activity (type IV collagenase). JHF cells, those derived from individuals with the milder disease, showed partial activation. ISH-derived fibroblasts, those with the more severe and fatal disease, had virtually no active MMP-2 (FIG. 10). Notably, control cells, i.e., normal fibroblasts (lane 2) produced no to very little amounts of MMP-2 protein under the same conditions. Thus, JHF and ISH cells, unlike normal fibroblasts, secrete MMP-2 when plated on laminin-containing plates. Further, once secreted, the ISH cells could not activate MMP-2 while JHF cells were capable of activating approximately half of the MMP-2 proteins.

Members of the laminin family of heterotrimeric glycoproteins, containing α-, β- and γ-chains are major constituents of basement membranes, which are extracellular matrices (ECM) found in close contact with individual cells and cell layers (Jones et al. 2001, supra). Acting through specific receptors, laminin is crucial for the formation of direct contacts between the ECM and cells. Inherited defects in laminins are associated with human disease (McGowan and Marinkovich, Micros Res. Tech. 2000;51:262-279). For example, epidermolysis bullosa letalis (MIM 226700) is caused by mutations in any of the three laminin-5 associated glycoproteins, α3 (LAMA3), β3 (LAMB3), or α2 (LAMC2), (Pulkkinen and Uitto, Matrix Biol. 1999;18:29-42). In addition, and beyond their structural roles, laminins help control cellular activities by allowing the bridging together of information between adjacent cells through interaction with cell surface receptors. Strikingly, mutations in the epithelial expressed, heterodimer-linked laminin receptor proteins, integrin-β4 gene (ITGB4) and integrin-α6 gene (ITGA6) cause disease in a subset of these patients but with additional gastrointestinal manifestations, epidermolysis bullosa with pyloric atresia (MIM 226730), (Vidal et al., Nat. Genet. 1995; 10:229-234; Ruzzi et al., J. Clin. Invest. 1997;99:2826-2831). Mutations in any component of dystroglycan, a major receptor for α2-laminins in the muscle sarcolemma results in a range of muscular dystrophies which can be characterized by loss of basement membrane architecture and function (Colognato and Yurchenco, Dev. Dyn. 2000;218:213-34). Also, basement membrane assembly is believed to be regulated by epithelial-mesenchymal interactions (Lonai, J Anat 2003;202:43-50). Accordingly, without being bound to any specific theory, the unexpected findings of the present study, that CMG-2 mutations found in JHF and ISH patients disrupt laminin-binding and reduces MMP-2 activation, not only provides a potential pathogenic mechanism for JHF and ISH, but has important implications for the treatment of non-JHF or non-ISH patients suffering from conditions associated with these disorders, such as osteoporosis and arthritis.

Patents, patent applications, publications, product descriptions, and protocols are cited throughout this application, the disclosures of which are incorporated herein by reference in their entireties for all purposes.

1. A method for diagnosing a disease, disorder, or condition associated with at least one of osteoporosis, osteopenia, osteolysis, and arthritis, in a subject, which method comprises detecting a variant capillary morphogenesis gene-2 (CMG-2) gene in the subject.

2. The method of claim 1, wherein the disorder or condition is infantile systemic hyalinosis (ISH) or juvenile hyaline fibromatosis (JHF).

3. The method of claim 1, wherein the variant has a mutation or polymorphism which is a member of the group consisting of a deletion, an insertion, a substitution, and combinations thereof.

4. The method of claim 3, wherein the mutation or polymorphism is in a coding region of the gene.

5. The method of claim 3, wherein the mutation or polymorphism is in a non-coding region of the gene.

6. The method of claim 4, wherein the mutation or polymorphism results in a variant of a CMG-2 protein having the sequence of SEQ ID NO:3, 5, 6, or 7.

7. The method of claim 1, wherein the mutation or polymorphism results in the deletion of a segment comprising more than one amino acid of SEQ ID NO:3, 5, 6, or 7.

8. The method of claim 6, wherein the mutation in the CMG-2 protein is selected from the group consisting of
(a) E220X
(b) G105D
(c) L329R;
(d) P257insC; and
(e) 1189T.

9. The method of claim 4, wherein the polymorphism in the CMG-2 protein is selected from A357P and A322S.

10. The method of claim 8, wherein the mutation in the CMG-2 gene, having the sequence of SEQ ID NO:1, is selected from the group consisting of:
(a) G37632T;
(b) G17627A;
(c) T65089G;
(d) C88794CC; and
(e) T19288C.

11. The method of claim 9, wherein the polymorphism in the CMG-2 gene, having the sequence of SEQ ID NO:1, is selected from C88790G and C48964T.

12. A kit for diagnosing a disease, disorder, or condition associated with at least one of osteoporosis, osteopenia, osteolysis, and arthritis, comprising an oligonucleotide that specifically hybridizes to or adjacent to a site of mutation or polymorphism in a CMG-2 gene, and instructions for use.

13. The kit of claim 12, wherein the disorder or condition is JHF or ISH.

14. The kit of claim 12, wherein the site of mutation or polymorphism comprises a nucleotide selected from the group consisting of nucleotides 37632, 17627, 65098, 88794, 19288, 17700, 18352, 19400, 88790, 116113, 166226, and 48964.

15. The kit of claim 12, comprising at least one probe comprising the site of mutation or polymorphism.

16. The kit of claim 12, comprising a first oligonucleotide primer comprising at least 15 consecutive nucleotides of SEQ ID NO:1, and a second oligonucleotide primer comprising at least 15 consecutive nucleotides of a sequence complementary to SEQ ID NO:1.

17. The kit of claim 12, comprising a first primer comprising a nucleotide sequence selected from the group consisting of SEQ ID NOS: 26, 28, 30, 32, 34, 36, 38, 40, 42, 44, 46, 48, and 50, and a second primer selected from the group consisting of SEQ ID NOS: 27, 29, 31, 33, 35, 37, 39, 41, 43, 45, 47, 49, and 51.

18. A kit for diagnosing a disease, disorder, or condition associated with at least one of osteoporosis, osteopenia, osteolysis, and arthritis comprising an antibody that specifically recognizes a mutation or polymorphism in a CMG-2 protein; and instructions for use.

19. The kit of claim 18, wherein the disorder or condition is JHF or ISH.

20. The kit of claim 18, wherein the CMG-2 protein has the sequence of SEQ ID NO:3, 5, 6, or 7.

21. The kit of claim 20, wherein the mutation is selected from the group consisting of:
(a) E220X;
(b) G105D;
(c) L329R;
(d) P257insC; and
(e) 1189T.

22. The kit of claim 20, wherein the polymorphism is A357P or A322S.

23. A method for diagnosing a disease, disorder, or condition associated with at least one of osteoporosis, osteopenia, osteolysis, and arthritis in a subject, which method comprises assessing the level of expression or activity of wild-type CMG-2 having the sequence of SEQ ID NO:3, 5, 6, or 7 in the test subject and comparing it to the level of expression or activity of wild-type CMG-2 in a control subject, wherein a decreased level of expression is indicative of the disease, disorder, or condition.

24. The method of claim 23, wherein the disorder or condition is JHF or ISH.

25. The method of claim 23, wherein the level of expression is assessed by determining the amount of mRNA that encodes the wild-type CMG-2 in a biological sample.

26. The method of claim 23, wherein the level of expression of is assessed by determining the concentration of wild-type, full-length CMG-2 protein in a biological sample.

27. The method of claim 23, wherein the level of activity is assessed by determining the level of fibroblasts in a biological sample capable of binding to laminin.

28. The method of claim 23, wherein the level of activity is assessed by determining the level of fibroblasts in a biological sample capable of producing mature matrix metalloproteinase 2 (MMP-2).

29. A method for treating a disease, disorder, or condition associated with at least one of osteoporosis, osteopenia, osteolysis, and arthritis, which method comprises administering to a patient in need of such treatment an effective amount of an agent that provides CMG-2 activity, in association with a pharmaceutically acceptable carrier.

30. The method of claim 29, wherein the disorder or condition is JHF or ISH.

31. The method of claim 29, wherein the agent is wild-type CMG-2 protein having the sequence of SEQ ID NO:3, 5, 6, or 7.

32. The method of claim 30, wherein the agent is a gene encoding CMG-2 protein having the sequence of SEQ ID NO:2 or 4.

33. An isolated variant of CMG-2 having the sequence of SEQ ID NO:3, 5, 6, or 7, the variant comprising a mutation selected from the group consisting of:
(a) E220X
(b) G105D
(c) L329R;
(d) P257insC; and
(e) 1189T.

34. An isolated cell comprising a vector, which vector comprises a nucleic acid encoding the CMG-2 variant of claim 33, operatively associated with an expression control sequence.

35. The cell of claim 34, selected from a prokaryotic cell and an eukaryotic cell.

36. An isolated nucleic acid encoding the CMG-2 variant of claim 33.

37. An isolated oligonucleotide which specifically hybridizes to the nucleic acid of claim 36.

38. An isolated variant of CMG-2 having the sequence of SEQ ID NO:3, 5, 6, or 7, the variant comprising a polymorphism selected from A357P and A322S.

39. An isolated cell comprising a vector, which vector comprises a nucleic acid encoding the CMG-2 variant of claim 38, operatively associated with an expression control sequence.

40. The cell of claim 39, selected from a prokaryotic cell and an eukaryotic cell.

41. An isolated nucleic acid encoding the CMG-2 variant of claim 38.

42. An isolated oligonucleotide which specifically hybridizes to the nucleic acid of claim 41.
